(19) United States
(12) Patent Application Publication
(10) Pub. No.: US 2004/0033975 A1
(43) Pub. Date: Feb. 19, 2004

(54) Whole cell engineering using real-time metabolic flux analysis

(75) Inventors: Pengcheng Fu, Honolulu, HI (US); Martin Latterich, San Diego, CA (US); Michael Levin, San Diego, CA (US); Jing Wei, San Diego, CA (US); Jay M. Short, Rancho Santa Fe, CA (US)

Correspondence Address: GREGORY P. EINHORN, Fish & Richardson P.C., Suite 500, 4350 La Jolla Village Drive, San Diego, CA, 92122, US

(73) Assignee: Diversa Corporation, San Diego, CA

(21) Appl. No.: 10/264,989

(22) Filed: Oct. 1, 2002

Related U.S. Application Data
(60) Provisional application No. 60/326,655, filed on Oct. 1, 2001.
(60) Provisional application No. 60/326,654, filed on Oct. 1, 2001.
(60) Provisional application No. 60/326,653, filed on Oct. 1, 2001.
(60) Provisional application No. 60/337,526, filed on Nov. 9, 2001.

(51) Int. Cl. A61K 48/00; C12Q 1/00; C12Q 1/68; G06F 19/00; G01N 33/48; G01N 33/50

(52) U.S. Cl. 514/44; 435/4; 435/6; 702/19

(57) ABSTRACT

The invention provides methods for whole cell engineering of new and modified phenotypes by using “on-line” or “real-time” metabolic flux analysis. The invention provides a method for whole cell engineering of new or modified phenotypes by using real-time metabolic flux analysis by making a modified cell by modifying the genetic composition of a cell and culturing the modified cell to generate a plurality of modified cells and measuring at least one metabolic parameter of the cell by monitoring the cell culture of in real time. The invention also provides articles comprising machine-readable medium including machine-executable instructions and systems, e.g., computer systems, to practice the methods of the invention.

This application claims the benefit of U.S. Provisional Application Nos. 60/326,655, 60/326,654 and 60/326,653 all filed Oct. 1, 2001, the entire disclosure of which is incorporated by reference as part of this application.

### TECHNICAL FIELD

The present invention is generally directed to the fields of whole cell engineering, cell biology and molecular biology. In particular, the invention is directed to methods and systems for whole cell engineering of new and modified phenotypes by using metabolic flux analysis. The invention also provides articles comprising machine-readable medium including machine-executable instructions and systems, e.g., computer systems, to practice the methods of the invention.

### BACKGROUND

Whole cell metabolic flux analysis is a “horizontal” or “holistic” approach to study the metabolism, or “metabolome,” of an organism. A whole cell “horizontal” metabolome approach studies the expression and function of all of the genes of an organism simultaneously. By using this whole cell approach to study a cell's metabolism, it is possible to get a complete snapshot of the whole cell's transcriptome (the expressed transcripts, or mRNA messages) and proteome (the expressed polypeptides). However, such snapshots are static pictures of one aspect of a cell's physiology and metabolism.

### SUMMARY

The present invention is in part based on the recognition that development of a means to dynamically monitor many different parameters in a cell culture would be much more effective in detecting new or altered cell phenotypes and other properties and cell growth conditions than mere static data. Accordingly, this invention provides, among others, methods for whole cell engineering of new or modified phenotypes by using real-time metabolic flux analysis. Any phenotype can be added or altered using the systems and methods of the invention. The invention also provides articles comprising machine-readable medium including machine-executable instructions and systems, e.g., computer systems, to practice the methods of the invention.

In one aspect, the method comprise the following steps: (a) making a modified cell by modifying the genetic composition of a cell; (b) culturing the modified cell to generate a plurality of modified cells; (c) measuring at least one metabolic parameter of the cell by monitoring the cell culture of step (b) in real time; and, (d) analyzing the data of step (c) to determine if the measured parameter differs from a comparable measurement in an unmodified cell under similar conditions, thereby identifying an engineered phenotype in the cell using real-time metabolic flux analysis.

In one aspect, the genetic composition of the cell is modified by a method comprising addition of a nucleic acid to a cell. One or more nucleic acids can be added at the same time, or, in series. The genetic composition of the cell can be modified by addition of a nucleic acid heterologous to the cell, or, a nucleic acid homologous to the cell. The homologous nucleic acid can comprise a modified homologous nucleic acid, such as a modified homologous gene. The coding sequence or transcriptional regulatory sequence of a gene can be modified. Alternatively, the genetic composition of the cell can be modified by a method comprising deletion of a sequence or modification of a sequence in the cell. The genetic composition of the cell can be modified by a method comprising modifying or knocking out the expression of a gene.

Any phenotype can be added or modified. The genome, proteome and/or the metabolome of a cell can be altered using the systems and methods of the invention. Any phenotype can be specifically targeted for change or addition.

For example, specific heterologous genes can be inserted or specific homologous genes can be stochastically or non-stochastically modified. For example, the newly engineered phenotype can be, e.g., an increased or decreased expression or amount of a polypeptide, an increased or decreased amount of an mRNA transcript, an increased or decreased expression of a gene, an increased or decreased resistance or sensitivity to a toxin, an increased or decreased resistance use or production of a metabolite, an increased or decreased uptake of a compound by the cell, an increased or decreased rate of metabolism, and an increased or decreased growth rate.

In one aspect, the methods further comprise analyzing gene expression from un-sequenced organisms. For example this can be accomplished with the help of techniques like MEGASORT™ or LEAD™.

Exemplary phenotypes that can be added or altered comprise: increased or de novo production of an antibiotic (erythromycin, ampicillin, tetracycline, penicillin and the like); increased or de novo production of acetic acid; increased or de novo solvent resistance; and the like. One exemplary strain “improved” by the methods of the invention produce a free acetic acid; wherein the strain has resistance to the solvent used in the removal of the acetic acid.

As noted above, in one aspect, gene expression from un-sequenced organisms are analyzed. These techniques allow the ultra-large scale hybridization of two cDNA samples. These techniques also allow the sorting or analysis of cDNA species that-are differentially expressed between the two samples. Subsequent cloning and sequence analysis of differentially expressed genes can be performed. The information obtained in this aspect of the invention can be cluster-analyzed by software, e.g., GENESPRING™ software. The information obtained in this aspect of the invention can be relayed to appropriate databases and further compared or analyzed. This technology is also of use to study differential expression of low abundance mRNA species that are currently not possible via gene-chip based approaches.

In one aspect, the invention provides a bacterial strain that produces a free acetic acid that is resistant to a solvent used in the removal of acetic acid. Mutations that enhance solvent resistance or acetic acid productions are generated and monitored in cell culture using the systems and methods of the invention. To allow engineering of a strain that combines both desirable traits, gene expression analysis using the methods of the invention can correlate gene expression patterns with solvent resistance and/or acetic acid production. This is a targeted genetics approach to create a strain with both enhanced acetic acid production and solvent resistance.

The newly engineered phenotype can be a stable phenotype. In another aspect, it can be a transient or an inducible phenotype. In one aspect, modifying the genetic composition of a cell comprises insertion of a construct into the cell, wherein construct comprises a nucleic acid operably linked to a constitutively active promoter. Alternatively, modifying the genetic composition of a cell can comprise insertion of a construct into the cell, wherein construct comprises a nucleic acid operably linked to an inducible promoter. The nucleic acid added to the cell can be stably inserted into the genome of the cell. Alternatively, the nucleic acid added to the cell can propagate as an episome in the cell.

In one aspect, the nucleic acid added to the cell can encode a peptide or a polypeptide. The polypeptide can comprise a homologous polypeptide, such as a modified homologous polypeptide. Alternatively, the polypeptide can comprise a heterologous polypeptide. The nucleic acid added to the cell can encode a transcript comprising a sequence that is antisense to a homologous transcript. In one aspect, modifying the genetic composition of the cell can comprise increasing or decreasing the expression of an mRNA transcript. Modifying the genetic composition of the cell can comprise increasing or decreasing the expression of a polypeptide, a lipid, a mono- or poly-saccharide or a nucleic acid.

In one aspect, modifying the homologous gene can comprise knocking out expression of the homologous gene. Modifying the homologous gene can comprise increasing the expression of the homologous gene. The gene modification can be random, or stochastic, or, non-random, or targeted, i.e., non-stochastic.

In an exemplary non-stochastic gene modification, a gene to be inserted into a cell to modify a phenotype can be a heterologous gene or a sequence-modified homologous gene, wherein the sequence modification is made by a method comprising the following steps: (a) providing a template polynucleotide, wherein the template polynucleotide comprises a homologous gene of the cell (it can also be a heterologous gene that you wish to modify); (b) providing a plurality of oligonucleotides, wherein each oligonucleotide comprises a sequence homologous to the template polynucleotide, thereby targeting a specific sequence of the template polynucleotide, and a sequence that is a variant of the homologous gene; (c) generating progeny polynucleotides comprising non-stochastic sequence variations by replicating the template polynucleotide of step (a) with the oligonucleotides of step (b), thereby generating polynucleotides comprising homologous gene sequence variations. One variation of this method has been termed “gene site-saturation mutagenesis,” “site-saturation mutagenesis,” “saturation mutagenesis” or simply “GSSM,” and is described in further detail, below. It can be used in combination with other mutagenization processes. See, e.g., U.S. Pat. Nos. 6,171,820; 6,238,884.

Another exemplary non-stochastic gene modification process comprises introduction of two or more related polynucleotides into a suitable host cell such that a hybrid polynucleotide is generated by recombination and reductive reassortment. For example, the sequence modification of the gene to be modified (e.g., the heterologous gene or homologous gene) is made by a method comprising the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations. One variation of this method has been termed “synthetic ligation reassembly,” or simply “SLR,” and is described in further detail, below. It can be used in combination with other mutagenization processes. See, e.g., U.S. Pat. No. 6,171,820.

Any cell can be engineered by the methods the invention, including, e.g., prokaryotic cells and eukaryotic cells. Bacteria, Archaebacteria, fungi, yeast, plant cells, insect cells, mammalian cells, including human cells, without limitation, can be engineered by the methods the invention. Furthermore, intracellular parasites, bacteria, viruses can be “indirectly” engineered by culturing and monitoring of eukaryotic cells by the methods the invention, including, e.g., immunodeficiency viruses, e.g., HIV, oncoviruses, mycobacteria, protozoan organisms (e.g., trypanosomes, such as Trypanosoma rangeli), plasmodium (e.g., Plasmodium falciparum), toxoplasmosis (e.g., Toxoplasma gondii), Leishmania, and the like.

The method can further comprising selecting a cell comprising a newly engineered phenotype. The selected cell can be isolated. The method can further comprise culturing the selected or isolated cell, thereby generating a new cell strain or cell line comprising a newly engineered phenotype. The methods can further comprise isolating a cell comprising a newly engineered phenotype.

In practicing the methods of the invention, any metabolic parameter can be measured. In one aspect, several different metabolic parameters are evaluated in the cell culture. The metabolic parameters can be measured at the same time or sequentially. One exemplary metabolic parameter is rate of cell growth, which can be measured by, e.g., a change in optical density of the cell culture. Another exemplary metabolic parameter measured comprises a change in the expression of a polypeptide. Changes in the expression of the polypeptide can be measured by any method, e.g., a one-dimensional gel electrophoresis, a two-dimensional gel electrophoresis, a tandem mass spectography, an RIA, an ELISA, an immunoprecipitation and a Western blot.

In one aspect, the measured metabolic parameter comprises a change in expression of at least one transcript, or, the expression of a transcript of a newly introduced gene. The change in expression of the transcript can be measured by hybridization, quantitative amplification, Northern blot and the like. The transcript expression can be measured by hybridization of a sample comprising transcripts of a cell or nucleic acid representative of or complementary to transcripts of a cell by hybridization to immobilized nucleic acids on an array.

In one aspect, the measured metabolic parameter comprises a measurement of a metabolite, including primary and secondary metabolites. For example, the measured metabolic parameter can comprise an increase or a decrease in a primary or a secondary metabolite. The secondary metabolite can be selected from the group consisting of a glycerol and a methanol. The measured metabolic parameter can comprise an increase or a decrease in an organic acid, such as an acetate, butyrate, succinate, oxaloacetate, fumarate, alpha-ketoglutarate or phosphate.

In one aspect, the measured metabolic parameter comprises an increase or a decrease in intracellular pH, or, extracellular pH in a culture medium. The increase or a decrease in intracellular pH can be measured by intracellular application of a dye; the change in fluorescence of the dye can be measured over time. In one aspect, the measured metabolic parameter comprises gas exchange rate measurements.

In one aspect, the measured metabolic parameter comprises an increase or a decrease in synthesis of DNA or RNA over time. The increase or a decrease in synthesis, or accumulation, or decay, of DNA or RNA over time can be measured by intracellular application of a dye; the change in fluorescence of the dye can be measured over time.

In one aspect, the measured metabolic parameter comprises an increase or a decrease in uptake of a composition. The composition can be a metabolite, such as a monosaccharide, a disaccharide, a polysaccharide, a lipid, a nucleic acid, an amino acid and a polypeptide. The saccharide, disaccharide or polysaccharide can comprise a glucose or a sucrose. The composition can also be an antibiotic, a metal, a steroid and an antibody.

In one aspect, the measured metabolic parameter comprises an increase or a decrease in the secretion of a byproduct or a secreted composition of a cell. The byproduct or secreted composition can be a toxin, a lymphokine, a polysaccharide, a lipid, a nucleic acid, an amino acid, a polypeptide and an antibody.

In one aspect of the methods, the real time monitoring simultaneously measures a plurality of metabolic parameters. The real time monitoring of a plurality of metabolic parameters can comprise use of a Cell Growth Monitor device. The Cell Growth Monitor device can be a Wedgewood Technology, Inc., Cell Growth Monitor model 652, or similar model or variation thereof In one aspect, the real time simultaneous monitoring measures uptake of substrates, levels of intracellular organic acids and levels of intracellular amino acids. The real time simultaneous monitoring can measure: uptake of glucose; levels of acetate, butyrate, succinate, oxaloacetate, fumarate, alpha-ketoglutarate or phosphate; and, levels of intracellular natural amino acids.

In one aspect, the method further comprises use of a computer-implemented program to real time monitor the change in measured metabolic parameters over time. The computer-implemented program can comprise a computer-implemented method. The computer-implemented method can comprise metabolic network equations. These computer-implemented method can also comprise a pathway analysis, an error analysis, such as a weighted least squares solution, and a flux estimation. The computer-implemented method can further comprise a preprocessing unit to filter out the errors for the measurement before the metabolic flux analysis.

The invention provides methods comprising: culturing cells in a controllable cell environment; measuring at least one metabolic parameter to obtain at least two different measurements in real time during the culturing; processing the two different measurements to determine a rate of change in the metabolic parameter in real time during the culturing; and using the rate of change in a known metabolic network of the cells to determine a real-time metabolic flux distribution in the cells during the culturing.

In one aspect, the controllable cell environment comprises a fermentor or a bioreactor. The controllable cell environment can comprise a flask, a plate, a capillary tube, a test tube, a biomatrix or an artificial organ. The controllable cell environment can comprise parasitic systems (parasites), symbionts, feeder layers in cell cultures or artificial organs, and the like. In one aspect, the controllable cell environment comprises a plurality of microbioreactors, e.g., as sets of 48 to 96 microbioreactors in a microtiter plate-like arrangement.

In one aspect, the measured metabolic parameter comprises a gas or a volatile composition, such as oxygen, methanol, hydrogen, or ethanol or a combination thereof. The gas can be measured by an on-line mass spectrometer.

In one aspect, the measured metabolic parameter comprises a substrate, a metabolite or a small compound, such as a saccharide, e.g., glucose. The substrate, a metabolite or a small compound, e.g., glucose, can be measured by an on-line mass spectrometer or bio-analyzer.

In one aspect, the measured metabolic parameter comprises an organic acid, such as acetate, butyrate, succinate, oxaloacetate, fumarate, alpha-ketoglutarate, phosphate or a combination thereof The organic acid can be measured by an on-line HPLC, mass spectrograph, infrared spectrograph or equivalent devices.

The method can further comprise adjusting an operating parameter of the controllable cell environment based on the determined real-time metabolic flux distribution to change the culturing condition of the cell or cell culture to modify the metabolic flux distribution during the culturing. In one aspect, the operating parameter is adjusted to direct the metabolic flux distribution towards a desired distribution. The operating parameter can comprise a substrate supply to the controllable cell environment. The metabolic parameter or the operating parameter can comprise a temperature of the controllable cell environment, an intracellular pH value inside the controllable cell environment, a gas exchange rate inside the controllable cell environment for one or more gases produced during the culturing, a nutrient supply to the controllable cell environment, cell density in the controllable cell environment and the like. The cell density in the controllable cell environment can be monitored by a cell growth monitor device. In one aspect, the cells are cultured in a liquid medium and the cell density is monitored by measuring optical density of the cell culture.

The method can further comprise modifying a genetic composition of one or more initial cells of the cell culture prior to the culturing. The genetic modifying can be based on information obtained from a real-time metabolic flux distribution in an initial cell or cell culture, and wherein the real-time metabolic flux distribution is obtained by measuring a selected metabolic parameter of one initial cell to obtain at least two different measurements in real time during culturing of the initial cell or cell culture, processing the two different measurements to determine a rate of change in the selected metabolic parameter in real time, and, using the rate of change in a known initial metabolic network for the initial cell or cell culture to determine the real-time metabolic flux distribution in the initial cell or cell culture.

In one aspect, the modifying of the genetic composition comprises adding a nucleic acid of an initial cell or cell culture. The modifying of the genetic composition can comprise altering a nucleic acid of an initial cell or cell culture. The modifying of the genetic composition can comprise using an optimized directed evolution system to generate evolved chimeric sequences. The modifying of the genetic composition can comprise knocking out an expression of a selected gene.

In one aspect, the modifying of the genetic composition further comprises establishing the known metabolic network for the cell or cell culture by using information from, e.g., genomic, proteomics, metabolomics, bioinformatics, stoichiometry, microbiology and/or biochemical engineering knowledge and the like. The method can further comprise obtaining information from transcriptome and proteome data of the selected cell, and, combining the information with the real-time metabolic flux distribution in the selected cell to design a metabolic engineering process.

The method can further comprise providing a computer for processing in real time the two different measurements and determining the real-time metabolic flux distribution in the selected cell during the culturing. The method can further comprise using the computer to retrieve information from at least one of a group consisting of bioinformatics, stoichiometry, microbiology, and biochemical engineering knowledge in establishing the known metabolic network for the selected cell. Any biologically reproducing system is considered a cell and can be used, e.g., plasmids, prions, phage, virions (e.g., DNA and RNA viruses) and the like, all prokaryotic, eukaryotic and archaeal cells e.g., bacterial cells, insect cells, plant cells, yeast cells and mammalian cells.

The invention provides an article comprising a machine-readable medium including machine-executable instructions, the instructions being operative to cause a machine to: electronically interface with a plurality of measuring devices coupled to a controllable cell environment to, in real time, obtain electronic data indicative of a plurality of metabolic parameters or conditions of cell culturing therein; process the electronic data, in real time, to produce values for a set of selected metabolic parameters or conditions indicative of real-time metabolic properties of the cultured cells in the controllable cell environment; retrieve information from at least one database comprising data on a metabolic network for the cultured cells; and, use the metabolic network and values for the set of selected metabolic parameters or conditions to determine a real-time metabolic flux distribution in the cultured cells. Any biologically reproducing system is considered a cell and can be used, e.g., plasmids, prions, phage, virions (e.g., DNA and RNA viruses) and the like, all prokaryotic, eukaryotic and archaeal cells e.g., bacterial cells, insect cells, plant cells, yeast cells and mammalian cells.

In one aspect, the data on the metabolic network for the cultured cells comprises a stoichiometry matrix for the cultured cells. The stoichiometry matrix can comprise a representation of a metabolic network of the cultured cells. The stoichiometry matrix can define the presence or absence of one or more metabolic pathway associations, including all the known metabolic pathways of a cell. The stoichiometry matrix can be represented by a stoichiometry coefficient A, wherein A·x=r, and r is a measurement vector representing on-line real-time measurements of the metabolic parameters and x is a flux vector having the units mmol/hour dry cell weight (DCW).

In one aspect, r the measurement vector represents the specific input and output rates of enzymes in a metabolic pathway of the cultured cells. The data on the metabolic network for the cultured cells can be, e.g., bioinformatics, stoichiometry, genomics, proteomics, metabolomics, microbiology and biochemical pathway and enzyme kinetics knowledge, and the like. The metabolic network for the selected cell can comprise a set of stoichiometric equations for metabolites in the selected cell.

In one aspect, the instructions are further operative to cause the machine to present the real-time metabolic flux distribution in the selected cell in a display device coupled to the machine. The instructions can be further operative to cause the machine to present the real-time metabolic flux distribution in a graphical form in the display device. The graphical form in the display device can show internal metabolic fluxes over a map of relevant metabolic pathways in the selected cell. The instructions can be further operative to cause the machine to present the real-time metabolic flux distribution in a graphical form in the display device. In one aspect, the instructions are operable in at least one operating system selected from a group consisting of Windows, UNIX, Linux, and MacOS. In one aspect, the instructions are further operative to cause the machine to: obtain at least two different measurements in real time during the culturing; processing the two different measurements to determine a rate of change in a metabolic parameter in real time during the culturing; and, using the rate of change in the metabolic network to determine the real-time metabolic flux distribution in the cultured cells.

The invention provides a system (e.g., system having a computer), comprising: (a) a controllable cell environment for culturing cells, wherein the operating conditions for culturing the cells is controllable in response to a control command; (b) a sensing subsystem coupled to the controllable cell environment to obtain, in real time during the culturing, measurements associated with culturing of the cells in the controllable cell environment; and, (c) a system controller coupled to the sensing subsystem to receive, in real time during the culturing, the measurements and operable to process the measurements to produce a real-time metabolic flux distribution in the cultured cells. In one aspect, the operating conditions for culturing the cells is based on a real-time metabolic flux distribution in the cultured cells.

The system can further comprise use of the real-time metabolic flux distribution of step (c) to determine the operating conditions for culturing the cells. The controllable cell environment of the system can comprise a fermentor or a bioreactor, a flask, a plate, a capillary tube, a test tube, a biomatrix or an artificial organ. The controllable cell environment of the system can comprise a plurality of microbioreactors.

In one aspect, the controllable cell environment comprises a cell growth monitor device. The cell growth monitor device can measure cell density, e.g. cell density in a liquid culture medium. In one aspect, the cells are cultured in a liquid medium and the cell density is monitored by on-line measurement of optical density of the cell culture.

In one aspect, the sensing subsystem comprises a device that detects an mRNA transcript. The device can be configured to operate based on Northern blots, quantitative amplification reactions, hybridization to arrays and the like. In another aspect, the sensing subsystem comprises a device that detects and determines the levels of a gas, an organic acid, a polypeptide, a peptide, amino acid, a polysaccharide, a lipid or a combination thereof. The device can comprise a nuclear magnetic resonance (NMR) device, a spectrophotometer, a high performance liquid chromatography (HPLC) device, a thin layer chromatography device, a hyperdiffusion chromatography device and the like. The device can be configured to operate based on an immunological method.

In one aspect, the organic acid detected and/or measured by the sensing subsystem is acetate, butyrate, succinate, oxaloacetate, fumarate, alpha-ketoglutarate, phosphate or a combination thereof. In one aspect, the gas or volatile composition detected and/or measured by the sensing subsystem is oxygen, methanol, hydrogen, ethanol or a combination thereof.

In one aspect, the sensing subsystem comprises a device that monitors a primary metabolite, a secondary metabolite or a combination thereof. The primary metabolite or secondary metabolite can comprise ethanol, methanol, glucose or a combination thereof.

In one aspect, the sensing subsystem comprises a device that detects an intracellular pH value in the controllable cell environment. In one aspect, the sensing subsystem comprises a device that detects and identifies a phenotype. In one aspect, the sensing subsystem comprises a capillary array operable to monitor a composition in the selected cell. The sensing subsystem can also comprise a device that retrieves a liquid sample from the controllable cell environment and measures a chemical constituent in the liquid sample. The sensing subsystem can also comprise a device that retrieves a gas sample from the controllable cell environment and measures chemical constituents in the gas sample.

In one aspect, the system controller comprises: one or more electronic interfaces coupled to the sensing subsystem to retrieve data representing the measurements; and, a computer coupled to the electronic interfaces to receive the data, wherein the computer is programmed to process the data to produce the real-time metabolic flux distribution in the cultured cells.

In one aspect, the computer is programmed to process the data, in real time, to produce values for a set of selected parameters indicative of real-time metabolic properties of the cultured cells in the controllable cell environment. The computer can be programmed to retrieve information from at least one database comprising data on a metabolic network for the cultured cells. The data on the metabolic network for the cultured cells can be from bioinformatics, stoichiometry, genomics, proteomics, metabolomics, microbiology and biochemical pathway and enzyme kinetics knowledge, and from databases comprising such information. In one aspect, the computer is programmed to use the metabolic network data and the values for the set of selected parameters indicative of real-time metabolic properties of the cultured cells to determine the real-time metabolic flux distribution in the cultured cells. The computer may connected to a local or a remote electronic device that stores information for metabolic flux analysis to retrieve such information for data processing. Such a electronic device may be a storage device in another computer or a server in a computer network and may be connected via a communication link which may be established via the Internet. The system controller may access information from various genetic and biochemistry databases including an on-line genomic database.

The computer can be further programmed to obtain at least two different measurements in real time during the cell culturing; process the two different measurements to determine a rate of change in a metabolic parameter in real time during the culturing; and/or use the rate of change in the metabolic network to determine the real-time metabolic flux distribution in the selected cell during the culturing, or any combination thereof. The computer can be configured to operate in at least one operating system, e.g., Windows, UNIX, Linux or MacOS.

In one aspect, the system controller further comprises a display device coupled to the computer. The system can further comprise a user interface allowing a user to view real-time on-line data, the results of the calculations, e.g., the MFA, real-time metabolic flux distribution, a stoichiometry matrix and the like. The computer can be further programmed to present the real-time metabolic flux distribution in a graphical form in the display device. The computer can be further programmed to present the graphical form such that internal metabolic fluxes are shown over a map of relevant metabolic pathways in the selected cell.

The system can further comprise a cell modification subsystem that operates to modify a genetic composition in a cell in the controllable cell environment in response to the real-time metabolic flux distribution produced by the system controller. The data on the metabolic network for the cultured cells can comprise a stoichiometry matrix for the cultured cells. The stoichiometry matrix can comprise a representation of a metabolic network of the cultured cells. The stoichiometry matrix can define the presence or absence of metabolic pathway associations. The stoichiometry matrix can be represented by a stoichiometry coefficient A, wherein A·x=r, and r is a measurement vector representing on-line real-time measurements of the metabolic parameters and x is a flux vector having the units mmol/hour dry cell weight (DCW). In one aspect, r the measurement vector represents the specific input and output rates of enzymes in a metabolic pathway of the cultured cells.

The invention provides methods for determining the optimal culture conditions for generating a desired product or a desired phenotype in cultured cells comprising: culturing cells in a controllable cell environment; measuring at least one metabolic parameter to obtain at least two different measurements in real time during the culturing; processing the two different measurements to determine a rate of change in the metabolic parameter in real time during the culturing; using the rate of change in a known metabolic network of the cells to determine a real-time metabolic flux distribution in the cells during the culturing; and, adjusting an operating parameter of the controllable cell environment based on the determined real-time metabolic flux distribution to change a culturing condition to modify the metabolic flux distribution during the culturing, thereby optimizing culture conditions for generating a desired product or a desired phenotype.

In yet another aspect, the invention provides a method for controlling a computer to perform an on-line metabolic flux analysis for cells under culturing in real time. The computer is first directed to access information on a proper metabolic network model for a selected cell under culturing for determining a metabolic flux distribution of the selected cell. The computer is next directed to receive data for determining the metabolic flux distribution. The received data is then used to compute specific rates of the selected cell. The metabolic network model is subsequently applied to the specific rates to determine the metabolic flux distribution. The data for the metabolic flux distribution is sent to data files for storage and to a computer display device for display. When the input data is changed, a new metabolic flux distribution is produced. Otherwise, the computer is directed to wait for a new set of data for determining a new metabolic flux distribution corresponding to the new set of data.

The invention provides a method for identifying proteins by differential labeling of peptides, the method comprising the following steps: (a) providing a sample comprising a polypeptide; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting the polypeptide into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated.

In one aspect, the sample of step (a) comprises a cell or a cell extract. The method can further comprise providing two or more samples comprising a polypeptide. One or more of the samples can be derived from a wild type cell and one sample can be derived from an abnormal or a modified cell. The abnormal cell can be a cancer cell. The modified cell can be a cell that is mutagenized &/or treated with a chemical, a physiological factor, or the presence of another organism (including, e.g. a eukaryotic organism, prokaryotic organism, virus, vector, prion, or part thereof), &/or exposed to an environmental factor or change or physical force (including, e.g., sound, light, heat, sonication, and radiation). The modification can be genetic change (including, for example, a change in DNA or RNA sequence or content) or otherwise.

In one aspect, the method further comprises purifying or fractionating the polypeptide before the fragmenting of step (c). The method can further comprise purifying or fractionating the polypeptide before the labeling of step (d). The method can further comprise purifying or fractionating the labeled peptide before the chromatography of step (e). In alternative aspects, the purifying or fractionating comprises a method selected from the group consisting of size exclusion chromatography, size exclusion chromatography, HPLC, reverse phase HPLC and affinity purification. In one aspect, the method further comprises contacting the polypeptide with a labeling reagent of step (b) before the fragmenting of step (c).

In one aspect, the labeling reagent of step (b) comprises the general formulae selected from the group consisting of: Z^{A}OH and Z^{B}OH, to esterify peptide C-terminals and/or Glu and Asp side chains; Z^{A}NH₂ and Z^{B}NH₂, to form amide bond with peptide C-terminals and/or Glu and Asp side chains; and Z^{A}CO₂H and Z^{B}CO₂H. to form amide bond with peptide N-terminals and/or Lys and Arg side chains; wherein Z^{A} and Z^{B} independently of one another comprise the general formula R-Z¹-A¹-Z²-A²-Z³-A³-Z⁴-A⁴-, Z¹, Z², Z³, and Z⁴ independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, and OB(OR)(OR¹), and R and R¹ is an alkyl group, A¹, A², A³, and A⁴ independently of one another, are selected from the group consisting of nothing or (CRR¹)n, wherein R, R¹, independently from other R and R¹ in Z¹ to Z⁴ and independently from other R and R¹ in A¹ to A⁴, are selected from the group consisting of a hydrogen atom, a halogen atom and an alkyl group; “n” in Z¹ to Z⁴, independent of n in A¹ to A⁴, is an integer having a value selected from the group consisting of 0 to about 51; 0 to about 41; 0 to about 31; 0 to about 21, 0 to about 11 and 0 to about 6.

In one aspect, the alkyl group (see definition below) is selected from the group consisting of an alkenyl, an alkynyl and an aryl group. One or more C—C bonds from (CRR¹)n can be replaced with a double or a triple bond; thus, in alternative aspects, an R or an R¹ group is deleted. The (CRR¹)n can be selected from the group consisting of an o-arylene, an m-arylene and a p-arylene, wherein each group has none or up to 6 substituents. The (CRR¹)n can be selected from the group consisting of a carbocyclic, a bicyclic and a tricyclic fragment, wherein the fragment has up to 8 atoms in the cycle with or without a heteroatom selected from the group consisting of an O atom, a N atom and an S atom.

In one aspect, two or more labeling reagents have the same structure but a different isotope composition. For example, in one aspect, Z^{A} has the same structure as Z^{B}, while Z^{A} has a different isotope composition than Z^{B}. In alternative aspects, the isotope is boron-10 and boron-11; carbon-12 and carbon-13; nitrogen-14 and nitrogen-15; and, sulfur-32 and sulfur-34. In one aspect, where the isotope with the lower mass is x and the isotope with the higher mass is y, and x and y are integers, x is greater than y.

In alternative aspects, x and y are between 1 and about 11, between 1 and about 21, between 1 and about 31, between 1 and about 41, or between 1 and about 51.

In one aspect, the labeling reagent of step (b) comprises the general formulae selected from the group consisting of: CD₃(CD₂)ₙOH/CH₃(CH₂)ₙOH, to esterify peptide C-terminals, where n=0, 1, 2 or y; CD₃(CD₂)ₙNH₂/CH₃(CH₂)ₙNH₂, to form amide bond with peptide C-terminals, where n=0, 1, 2 or y; and, D(CD₂)ₙCO₂H/H(CH₂)ₙCO₂H, to form amide bond with peptide N-terminals, where n=0, 1, 2 or y; wherein D is a deuteron atom, and y is an integer selected from the group consisting of about 51; about 41; about 31; about 21, about 11; about 6 and between about 5 and 51.

In one aspect, the labeling reagent of step (b) can comprise the general formulae selected from the group consisting of: Z^{A}OH and Z^{B}OH to esterify peptide C-terminals; Z^{A}NH₂/Z^{B}NH₂ to form an amide bond with peptide C-terminals; and, Z^{A}CO₂H/Z^{B}CO₂H to form an amide bond with peptide N-terminals; wherein Z^{A} and Z^{B} have the general formula R-Z¹-A¹-Z²-A²-Z³-A³-Z⁴-A⁴-; Z¹, Z², Z³, and Z⁴, independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, and OB(OR)(OR¹); A¹, A², A³, and A⁴, independently of one another, are selected from the group consisting of nothing and the general formulae (CRR¹)n, and, R and R¹ is an alkyl group.

In one aspect, a single C—C bond in a (CRR¹)n group is replaced with a double or a triple bond; thus, the R and R¹ can be absent. The (CRR¹)n can comprise a moiety selected from the group consisting of an o-arylene, an m-arylene and a p-arylene, wherein the group has none or up to 6 substituents. The group can comprise a carbocyclic, a bicyclic, or a tricyclic fragments with up to 8 atoms in the cycle, with or without a heteroatom selected from the group consisting of an O atom, an N atom and an S atom. In one aspect, R, R¹, independently from other R and R¹ in Z¹-Z⁴ and independently from other R and R¹ in A¹-A⁴, are selected from the group consisting of a hydrogen atom, a halogen and an alkyl group. The alkyl group (see definition below) can be an alkenyl, an alkynyl or an aryl group.

In one aspect, the “n” in Z¹-Z⁴ is independent of n in A¹-A⁴ and is an integer selected from the group consisting of about 51; about 41; about 31; about 21, about 11 and about 6. In one aspect, Z^{A} has the same structure a Z^{B} but Z^{A} further comprises x number of —CH₂— fragment(s) in one or more A¹-A⁴ fragments, wherein x is an integer. In one aspect, Z^{A} has the same structure a Z^{B} but Z^{A} further comprises x number of —CF₂— fragment(s) in one or more A¹-A⁴ fragments, wherein x is an integer. In one aspect, Z^{A} comprises x number of protons and Z^{B} comprises y number of halogens in the place of protons, wherein x and y are integers. In one aspect, Z^{A} contains x number of protons and Z^{B} contains y number of halogens, and there are x-y number of protons remaining in one or more A¹-A⁴ fragments, wherein x and y are integers. In one aspect, Z^{A} further comprises x number of —O— fragment(s) in one or more A¹-A⁴ fragments, wherein x is an integer. In one aspect, Z^{A} further comprises x number of —S— fragment(s) in one or more A¹-A⁴ fragments, wherein x is an integer. In one aspect, Z^{A} further comprises x number of —O— fragment(s) and Z^{B} further comprises y number of —S— fragment(s) in the place of —O— fragment(s), wherein x and y are integers. In one aspect, Z^{A} further comprises x-y number of —O— fragment(s) in one or more A¹ -A⁴ fragments, wherein x and y are integers.

In alternative aspects, x and y are integers selected from the group consisting of between 1 about 51; between 1 about 41; between 1 about 31; between 1 about 21, between 1 about 11 and between 1 about 6, wherein x is greater than y.

In one aspect, the labeling reagent of step (b) comprises the general formulae selected from the group consisting of: CH₃(CH₂)ₙOH/CH₃(CH₂)ₙ₊ₘOH, to esterify peptide C-terminals, where n=0, 1, 2, . . . , y; m=1, 2, . . . , y; CH₃(CH₂)ₙNH₂/CH₃(CH₂)ₙ₊ₘNH₂, to form amide bond with peptide C-terminals, where n=0, 1, 2, . . . , y; m=1, 2, . . . , y; and, H(CH₂)ₙCO₂H/H(CH₂)ₙ₊ₘCO₂H, to form amide bond with peptide N-terminals, where n=0, 1, 2, . . . , y; m=1, 2, . . . , y; wherein n, m and y are integers. In one aspect, n, m and y are integers selected from the group consisting of about 51; about 41; about 31; about 21, about 11; about 6 and between about 5 and 51.

In one aspect, the separating of step (e) comprises a liquid chromatography system, such as a multidimensional liquid chromatography or a capillary chromatography system. In one aspect, the mass spectrometer comprises a tandem mass spectrometry device. In one aspect, the method further comprises quantifying the amount of each polypeptide or each peptide.

The invention provides a method for defining the expressed proteins associated with a given cellular state, the method comprising the following steps: (a) providing a sample comprising a cell in the desired cellular state; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting polypeptides derived from the cell into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated, thereby defining the expressed proteins associated with the cellular state.

The invention provides a method for quantifying changes in protein expression between at least two cellular states, the method comprising the following steps: (a) providing at least two samples comprising cells in a desired cellular state; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting polypeptides derived from the cells into peptide fragments by enzymatic digestion or by non-enzymatic fragmentation; (d) contacting the labeling reagents of step (b) with the peptide fragments of step (c), thereby labeling the peptides with the differential labeling reagents, wherein the labels used in one same are different from the labels used in other samples; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which identifies from which sample each peptide was derived, compares the inputted sequence to a database of polypeptide sequences to identify the polypeptide from which the sequenced peptide originated, and compares the amount of each polypeptide in each sample, thereby quantifying changes in protein expression between at least two cellular states.

The invention provides a method for identifying proteins by differential labeling of peptides, the method comprising the following steps: (a) providing a sample comprising a polypeptide; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting the polypeptide into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by multidimensional liquid chromatography to generate an eluate; (f) feeding the eluate of step (e) into a tandem mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated.

The invention provides a chimeric labeling reagent comprising (a) a first domain comprising a biotin; and (b) a second domain comprising a reactive group capable of covalently binding to an amino acid, wherein the chimeric labeling reagent comprises at least one isotope. The isotope(s) can be in the first domain or the second domain. For example, the isotope(s) can be in the biotin.

In alternative aspects, the isotope can be a deuterium isotope, a boron-10 or boron-11 isotope, a carbon-12 or a carbon-13 isotope, a nitrogen-14 or a nitrogen-15 isotope, or, a sulfur-32 or a sulfur-34 isotope. The chimeric labeling reagent can comprise two or more isotopes. The chimeric labeling reagent reactive group capable of covalently binding to an amino acid can be a succimide group, an isothiocyanate group or an isocyanate group. The reactive group can be capable of covalently binding to an amino acid binds to a lysine or a cysteine.

The chimeric labeling reagent can further comprising a linker moiety linking the biotin group and the reactive group. The linker moiety can comprise at least one isotope. In one aspect, the linker is a cleavable moiety that can be cleaved by, e.g., enzymatic digest or by reduction.

The invention provides a method of comparing relative protein concentrations in a sample comprising (a) providing a plurality of differential small molecule tags, wherein the small molecule tags are structurally identical but differ in their isotope composition, and the small molecules comprise reactive groups that covalently bind to cysteine or lysine residues or both; (b) providing at least two samples comprising polypeptides; (c) attaching covalently the differential small molecule tags to amino acids of the polypeptides; (d) determining the protein concentrations of each sample in a tandem mass spectrometer; and, (d) comparing relative protein concentrations of each sample. In one aspect, the sample comprises a complete or a fractionated cellular sample.

In one aspect of the method, the differential small molecule tags comprise a chimeric labeling reagent comprising (a) a first domain comprising a biotin; and, (b) a second domain comprising a reactive group capable of covalently binding to an amino acid, wherein the chimeric labeling reagent comprises at least one isotope. The isotope can be a deuterium isotope, a boron-10 or boron-11 isotope, a carbon-12 or a carbon-13 isotope, a nitrogen-14 or a nitrogen-15 isotope, or, a sulfur-32 or a sulfur-34 isotope. The chimeric labeling reagent can comprise two or more isotopes. The reactive group can be capable of covalently binding to an amino acid is selected from the group consisting of a succimide group, an isothiocyanate group and an isocyanate group.

The invention provides a method of comparing relative protein concentrations in a sample comprising (a) providing a plurality of differential small molecule tags, wherein the differential small molecule tags comprise a chimeric labeling reagent comprising (i) a first domain comprising a biotin; and, (ii) a second domain comprising a reactive group capable of covalently binding to an amino acid, wherein the chimeric labeling reagent comprises at least one isotope; (b) providing at least two samples comprising polypeptides; (c) attaching covalently the differential small molecule tags to amino acids of the polypeptides; (d) isolating the tagged polypeptides on a biotin-binding column by binding tagged polypeptides to the column, washing non-bound materials off the column, and eluting tagged polypeptides off the column; (e) determining the protein concentrations of each sample in a tandem mass spectrometer; and, (f) comparing relative protein concentrations of each sample.

A method for identifying proteins by differential labeling of peptides, the method comprising the following steps: (a) providing a sample comprising a polypeptide; (b) providing a plurality of labeling reagents which differ in molecular mass but have the same or nearly identical or similar chromatographic retention properties and that have the same or nearly identical or similar ionization and detection properties in mass spectrographic analysis, wherein the differences in molecular mass are distinguishable by mass spectrographic analysis; (c) fragmenting the polypeptide into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated. In one aspect, the sample of step (a) comprises a cell or a cell extract.

The method can further comprise providing two or more samples comprising a polypeptide. In one aspect, one sample is derived from a wild type cell and one sample is derived from an abnormal or a modified cell. In one aspect, the abnormal cell is a cancer cell.

The method can further comprise purifying or fractionating the polypeptide before the fragmenting of step (c). The method can further comprise purifying or fractionating the polypeptide before the labeling of step (d). The method can further comprise purifying or fractionating the labeled peptide before the chromatography of step (e). In one aspect, the purifying or fractionating comprises a method selected from the group consisting of size exclusion chromatography, size exclusion chromatography, HPLC, reverse phase HPLC and affinity purification. The method can further comprise contacting the polypeptide with a labeling reagent of step (b) before the fragmenting of step (c).

In one aspect, the labeling reagent of step (b) comprises the general formulae selected from the group consisting of: Z^{A}OOH and Z^{B}OH, to esterify peptide C-terminals and/or Glu and Asp side chains; Z^{A}NH₂ and Z^{B}NH₂, to form amide bond with peptide C-terminals and/or Glu and Asp side chains; and Z^{A}CO₂H and Z^{B} CO₂H. to form amide bond with peptide N-terminals and/or Lys and Arg side chains; wherein Z^{A} and Z^{B} independently of one another comprise the general formula R-Z¹-A¹-Z²-A²-Z³-A³-Z⁴-A⁴-, Z¹, Z², Z³, and Z⁴ independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, and OB(OR)(OR¹), and R and R¹ is an alkyl group, A¹, A², A³, and A⁴ independently of one another, are selected from the group consisting of nothing or (CRR¹)n, wherein R, R¹, independently from other R and R¹ in Z¹ to Z⁴ and independently from other R and R¹ in A¹ to A⁴, are selected from the group consisting of a hydrogen atom, a halogen atom and an alkyl group; n in Z¹ to Z⁴, independent of n in A¹ to A⁴, is an integer having a value selected from the group consisting of 0 to about 51; 0 to about 41; 0 to about 31; 0 to about 21, 0 to about 11 and 0 to about 6.

In one aspect, the alkyl group is selected from the group consisting of an alkenyl, an alkynyl and an aryl group. In one aspect, one or more C—C bonds from (CRR¹)n are replaced with a double or a triple bond. In one aspect, an R or an R¹ group is deleted. In one aspect, (CRR¹)n is selected from the group consisting of an o-arylene, an m-arylene and a p-arylene, wherein each group has none or up to 6 substituents. In one aspect, (CRR¹)n is selected from the group consisting of a carbocyclic, a bicyclic and a tricyclic fragment, wherein the fragment has up to 8 atoms in the cycle with or without a heteroatom selected from the group consisting of an O atom, a N atom and an S atom.

In one aspect, two or more labeling reagents have the same structure but a different isotope composition. In one aspect, Z^{A} has the same structure as Z^{B}, but Z^{A} has a different isotope composition than Z^{B}. In one aspect, the isotope is boron-10 and boron-11, or, the isotope is carbon-12 and carbon-13, or, the isotope is nitrogen-14 and nitrogen-15, or, the isotope is sulfur-32 and sulfur-34. In one aspect, the isotope with the lower mass is x and the isotope with the higher mass is y, and x and y are integers, x is greater than y.

In one aspect, x and y are between 1 and about 11, between 1 and about 21, between 1 and about 31, between 1 and about 41, or between 1 and about 51.

In one aspect, the labeling reagent of step (b) comprises the general formulae selected from the group consisting of: CD₃(CD₂)ₙOH/CH₃(CH₂)ₙOH, to esterify peptide C-terminals, where n=0, 1, 2 or y; ii. CD₃(CD₂)ₙNH₂/CH₃(CH₂)ₙNH₂, to form amide bond with peptide C-terminals, where n=0, 1, 2 or y; and D(CD₂)ₙCO₂H/H(CH₂)ₙCO₂H, to form amide bond with peptide N-terminals, where n=0, 1, 2 or y; wherein D is a deuteron atom, and y is an integer selected from the group consisting of about 51; about 41; about 31; about 21, about 11; about 6 and between about 5 and 51.

In one aspect, the labeling reagent of step (b) comprises the general formulae selected from the group consisting of: Z^{A}OH and Z^{B}OH to esterify peptide C-terminals; Z^{A}NH₂/Z^{B}NH₂ to form an amide bond with peptide C-terminals; and Z^{A}CO₂H/Z^{B}CO₂H to form an amide bond with peptide N-terminals; wherein Z^{A} and Z^{B} have the general formula R-Z¹-A¹-Z²-A²-Z³-A³-Z⁴-A⁴-Z¹, Z², Z³, and Z⁴, independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, and OB(OR)(OR¹); A¹, A², A³, and A⁴, independently of one another, are selected from the group consisting of nothing and the general formulae (CRR¹)n, and, R and R¹ is an alkyl group.

In one aspect, a single C—C bond in a (CRR¹)n group is replaced with a double or a triple bond. In one aspect, R and R¹ are absent. In one aspect, (CRR¹)n comprises a moiety selected from the group consisting of an o-arylene, an m-arylene and a p-arylene, wherein the group has none or up to 6 substituents. In one aspect, the group comprises a carbocyclic, a bicyclic, or a tricyclic fragments with up to 8 atoms in the cycle, with or without a heteroatom selected from the group consisting of an O atom, an N atom and an S atom. In one aspect, R, R¹ , independently from other R and R¹ in Z¹-Z⁴ and independently from other R and R¹ in A¹-A⁴, are selected from the group consisting of a hydrogen atom, a halogen and an alkyl group. In one aspect, the alkyl group is selected from the group consisting of an alkenyl, an alkynyl and an aryl group. In one aspect, Z¹-Z⁴ is independent of n in A¹-A⁴ and is an integer selected from the group consisting of about 51; about 41; about 31; about 21, about 11 and about 6. In one aspect, Z^{A} has the same structure a Z^{B} but Z^{A} further comprises x number of —CH₂— fragment(s) in one or more A¹-A⁴ fragments, wherein x is an integer. In one aspect, Z^{A} has the same structure a Z^{B} but Z^{A} further comprises x number of —CF₂— fragment(s) in one or more A¹-A⁴ fragments, wherein x is an integer. In one aspect, Z comprises x number of protons and Z^{B} comprises y number of halogens in the place of protons, wherein x and y are integers. In one aspect, Z^{A} contains x number of protons and Z^{B} contains y number of halogens, and there are x-y number of protons remaining in one or more A¹-A⁴ fragments, wherein x and y are integers. In one aspect, Z^{A} further comprises x number of —O— fragment(s) in one or more A¹-A⁴ fragments, wherein x is an integer. In one aspect, Z^{A} further comprises x number of —S— fragment(s) in one or more A¹-A⁴ fragments, wherein x is an integer. In one aspect, Z^{A} further comprises x number of —O— fragment(s) and Z^{B} further comprises y number of —S— fragment(s) in the place of —O— fragment(s), wherein x and y are integers. In one aspect, Z^{A} further comprises x-y number of —O— fragment(s) in one or more A¹-A⁴ fragments, wherein x and y are integers. In one aspect, x and y are integers selected from the group consisting of between 1 about 51; between 1 about 41; between 1 about 31; between 1 about 21, between 1 about 11 and between 1 about 6, wherein x is greater than y.

In one aspect, the labeling reagent of step (b) comprises the general formulae selected from the group consisting of: CH₃(CH₂)ₙOH/CH₃(CH₂)ₙ₊ₘOH, to esterify peptide C-terminals, where n=0, 1, 2, . . . , y; m=1, 2, . . . , y; CH₃(CH₂)ₙNH₂/CH₃(CH₂)ₙ₊ₘNH₂, to form amide bond with peptide C-terminals, where n=0, 1, 2, . . . , y; m=1, 2, . . . , y; and, H(CH₂)ₙCO₂H/H(CH₂)ₙ₊ₘCO₂H, to form amide bond with peptide N-terminals, where n=0, 1, 2, . . . , y; m=1, 2, . . . , y; wherein n, m and y are integers.

In one aspect, n, m and y are integers selected from the group consisting of about 51; about 41; about 31; about 21, about 11; about 6 and between about 5 and 51. In one aspect, the separating of step (e) comprises a liquid chromatography system.

In one aspect, the liquid chromatography system comprises a multidimensional liquid chromatography. In one aspect, the mass spectrometer comprises a tandem mass spectrometry device.

The method can further comprise quantifying the amount of each polypeptide. The method can further comprise quantifying the amount of each peptide.

The invention provides methods for defining the expressed proteins associated with a given cellular state, the method comprising the following steps: (a) providing a sample comprising a cell in the desired cellular state; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting polypeptides derived from the cell into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated, thereby defining the expressed proteins associated with the cellular state.

The invention provides methods for quantifying changes in protein expression between at least two cellular states, the method comprising the following steps: (a) providing at least two samples comprising cells in a desired cellular state; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting polypeptides derived from the cells into peptide fragments by enzymatic digestion or by non-enzymatic fragmentation; (d) contacting the labeling reagents of step (b) with the peptide fragments of step (c), thereby labeling the peptides with the differential labeling reagents, wherein the labels used in one same are different from the labels used in other samples; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which identifies from which sample each peptide was derived, compares the inputted sequence to a database of polypeptide sequences to identify the polypeptide from which the sequenced peptide originated, and compares the amount of each polypeptide in each sample, thereby quantifying changes in protein expression between at least two cellular states.

The invention provides methods for identifying proteins by differential labeling of peptides, the method comprising the following steps: (a) providing a sample comprising a polypeptide; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting the polypeptide into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by multidimensional liquid chromatography to generate an eluate; (f) feeding the eluate of step (e) into a tandem mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated.

The invention provides chimeric labeling reagents comprising (a) a first domain comprising a biotin; and (b) a second domain comprising a reactive group capable of covalently binding to an amino acid, wherein the chimeric labeling reagent comprises at least one isotope. In one aspect, the isotope is in the first domain. In one aspect, the isotope is in the biotin. In one aspect, the isotope is in the second domain. In one aspect, the isotope is selected from the group consisting of a deuterium isotope, a boron-10 or boron-11 isotope, a carbon-12 or a carbon-13 isotope, a nitrogen-14 or a nitrogen-15 isotope and a sulfur-32 or a sulfur-34 isotope. In one aspect, the labeling reagent comprises two or more isotopes.

In one aspect, the reactive group capable of covalently binding to an amino acid is selected from the group consisting of a succimide group, an isothiocyanate group and an isocyanate group. In one aspect, the reactive group capable of covalently binding to an amino acid binds to a lysine or a cysteine. The chimeric labeling reagents can further comprise a linker moiety linking the biotin group and the reactive group. The linker moiety comprises at least one isotope. In one aspect, the linker is a cleavable moiety. In one aspect, the linker can be cleaved by enzymatic digest. In one aspect, the linker can be cleaved by reduction.

The invention provides methods of comparing relative protein concentrations in a sample comprising (a) providing a plurality of differential small molecule tags, wherein the small molecule tags are structurally identical but differ in their isotope composition, and the small molecules comprise reactive groups that covalently bind to cysteine or lysine residues or both; (b) providing at least two samples comprising polypeptides; (c) attaching covalently the differential small molecule tags to amino acids -of the polypeptides; (d) determining the protein concentrations of each sample in a tandem mass spectrometer; and, (d) comparing relative protein concentrations of each sample. In one aspect, the sample comprises a complete or a fractionated cellular sample. In one aspect, differential small molecule tags comprise a chimeric labeling reagent comprising (a) a first domain comprising a biotin; and, (b) a second domain comprising a reactive group capable of covalently binding to an amino acid, wherein the chimeric labeling reagent comprises at least one isotope. In one aspect, the isotope is selected from the group consisting of a deuterium isotope, a boron-10 or boron-11 isotope, a carbon-12 or a carbon-13 isotope, a nitrogen-14 or a nitrogen-15 isotope and a sulfur-32 or a sulfur-34 isotope. In one aspect, the chimeric labeling reagent comprises two or more isotopes. In one aspect, the reactive group capable of covalently binding to an amino acid is selected from the group consisting of a succimide group, an isothiocyanate group and an isocyanate group.

The invention provides methods of comparing relative protein concentrations in a sample comprising (a) providing a plurality of differential small molecule tags, wherein the differential small molecule tags comprise a chimeric labeling reagent comprising (i) a first domain comprising a biotin; and, (ii) a second domain comprising a reactive group capable of covalently binding to an amino acid, wherein the chimeric labeling reagent comprises at least one isotope; (b) providing at least two samples comprising polypeptides; (c) attaching covalently the differential small molecule tags to amino acids of the polypeptides; (d) isolating the tagged polypeptides on a biotin-binding column by binding tagged polypeptides to the column, washing non-bound materials off the column, and eluting tagged polypeptides off the column; (e) determining the protein concentrations of each sample in a tandem mass spectrometer; and, (f) comparing relative protein concentrations of each sample.

The invention provides a multidimensional micro liquid chromatography MS/MS (μLC-MS/MS) system comprising three-dimensional (3-D) microcapillary columns for liquid chromatograph (LC) separation of peptides comprising a configuration comprising a reverse phase (RP1) chromatograph, a strong cation exchange (SCX) chromatograph and a reverse phase (RP2) resin chromatograph. In one aspect of the multidimensional micro liquid chromatography MS/MS (μLC-MS/MS) system, the system is configured with the components of the system are in the following order: a reverse phase (RP1) chromatograph, followed by a strong cation exchange (SCX) chromatograph, followed by a reverse phase (RP2) resin chromatograph.

The details of one or more aspects of the invention are set forth in the accompanying drawings and the description below. Other features, objects, and advantages of the invention will be apparent from the description and drawings, and from the claims.

All publications, GenBank Accession references (sequences), ATCC Deposits, patents and patent applications cited herein are hereby expressly incorporated by reference for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows one embodiment of a cell engineering method based on real-time metabolic flux analysis.

FIG. 2A show one embodiment of a computer-implemented metabolic flux analysis process. FIGS. 2B through 2E further show various aspects and examples of the present invention.

FIG. 3 illustrates one embodiment of a cell growth system with an on-line sensing subsystem for monitoring the cell growth in real time, an on-line data processing mechanism for processing the measurements in real time, and a control mechanism for controlling the conditions of the cell growth where the control may be made in response to the real time measurements.

FIG. 4 shows one exemplary cell engineering process that may be carried out by using the system shown in FIG. 3.

FIG. 5 illustrates one implementation of a cell growth and engineering system in part based on the system shown in FIG. 3, where a cell modification subsystem is used to modify or engineer the cells according to real-time measurements of the cells under culturing in a controllable cell environment such as a fermentor or bioreactor.

FIG. 6 further shows one example of a cell modification subsystem that may be used in the system in FIG. 5.

FIG. 7 shows operations that may be carried out with the system in FIG. 5.

FIG. 8 shows one example of a graphic representation of the MFA results on a computer display.

FIG. 9 shows another embodiment of processing steps for real-time MFA-based cell growth and engineering based on the basic operation process in FIG. 2.

FIGS. 10A through 10H show exemplary implementations of the program in FIG. 9 by using the LABVIEW™ software.

FIG. 11 shows a display of the LABVIEW™ software for the output from the operations in FIG. 9.

FIG. 12 summarizes in table form matrix measurements for the analysis of A in calculating the metabolic flux of a S. cerevisaie system (FIG. 12, FIG. 12A (page 1), 12B (page 2) and 12C (page 3)), as described in detail in Example 2, below.

FIG. 13 summarizes in table form the results of a metabolic flux analysis for a S. cerevisaie system as described in detail in Example 2, below.

FIG. 14 summarizes in table form matrix measurements for the analysis of A in calculating the metabolic flux of an E. coli system (FIG. 14, FIG. 14A (page 1), 14B (page 2) and 14C (page 3)), as described in detail in Example 3, below.

FIG. 15 illustrates an exemplary multidimensional micro liquid chromatography MS/MS (μLC-MS/MS) configuration of an exemplary system of the invention.

FIG. 16 illustrates (as Step 1) an exemplary 3-D column preparation and sample loading and (as Step 2) a 3-D separation of an exemplary 3-D μLC MS/MS system of the invention.

FIG. 17 illustrates the biosynthetic pathway for the antibiotic puromycin.

FIG. 18 illustrates examples of the identifications for the pathway-related proteins after the pathway engineering. The peptides detected by proteomic analysis are highlighted.

FIGS. 19A through 19G illustrate methods and interpretation of LC-MS or LC-LC-MS quantitative proteomics data.

Like reference symbols in the various drawings indicate like elements.

### DETAILED DESCRIPTION

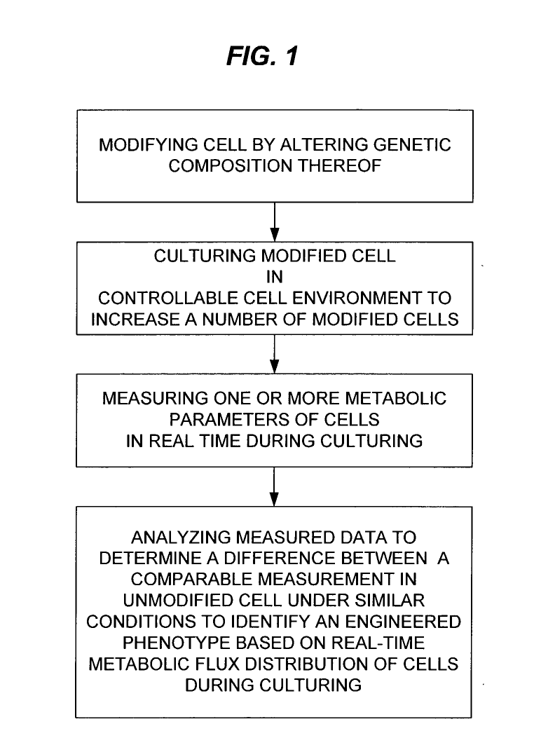

The invention provides, among others, novel methods and systems for whole cell engineering of new and modified phenotypes by using “on-line” or “real-time” metabolic flux analysis. FIG. 1 shows one embodiment for practicing the methods of the invention. As a first step, a cell is modified by changing the genetic composition of the cell. The modification can be random, i.e., stochastic, or, by non-stochastic methods, as described herein. Specific genes or specific metabolic pathways can be targeted for modification.

According to this embodiment, the second step of the methods of the invention comprises culturing the modified cell to generate a plurality of modified cells. This cell culturing may be performed in a controllable cell environment which may be controlled by an operator or through electronic and other control mechanisms. In general, the cells can be cultured by any means, for example, in cell culture, such as a tissue culture, by fermentation or tissue culture reactors, or in a cell growth monitor device.

The next step of the methods comprises measuring at least one metabolic parameter of the cell in real time. In one aspect, a plurality of metabolome parameters are simultaneously measured. Thus, one or several devices can be used to monitor and measure metabolic parameters. Such devices may be coupled to interact with the controllable cell environment to obtain the measurements and thus constitute a sensing subsystem in the cell systems of this invention. For example, a cell growth monitor device can measure a plurality of metabolic parameters of the cells in culture in real time. One example is the Wedgewood Technology, Inc. (San Carlos, Calif.), Cell Growth Monitor model 652™, as discussed below.

In addition, the methods comprise analyzing these data to determine if the measured parameters differ from a comparable measurement in an unmodified (or differently modified) cell under similar conditions, or, change over time, thereby identifying an engineered phenotype in the cell using real-time metabolic flux analysis. For example, the parameter can be higher, lower or change at a rate that differs from a wild type cell or otherwise unaltered cell or cell culture. It is not necessary to simultaneously monitor an unmodified cell or cell culture in real time to determine if and/or what phenotypic modifications result from the modification of the cell's genetic composition. Data and information already known can be used as a reference. The above process may be repeated until a cell or cell culture engineered with one or more desired properties is produced.

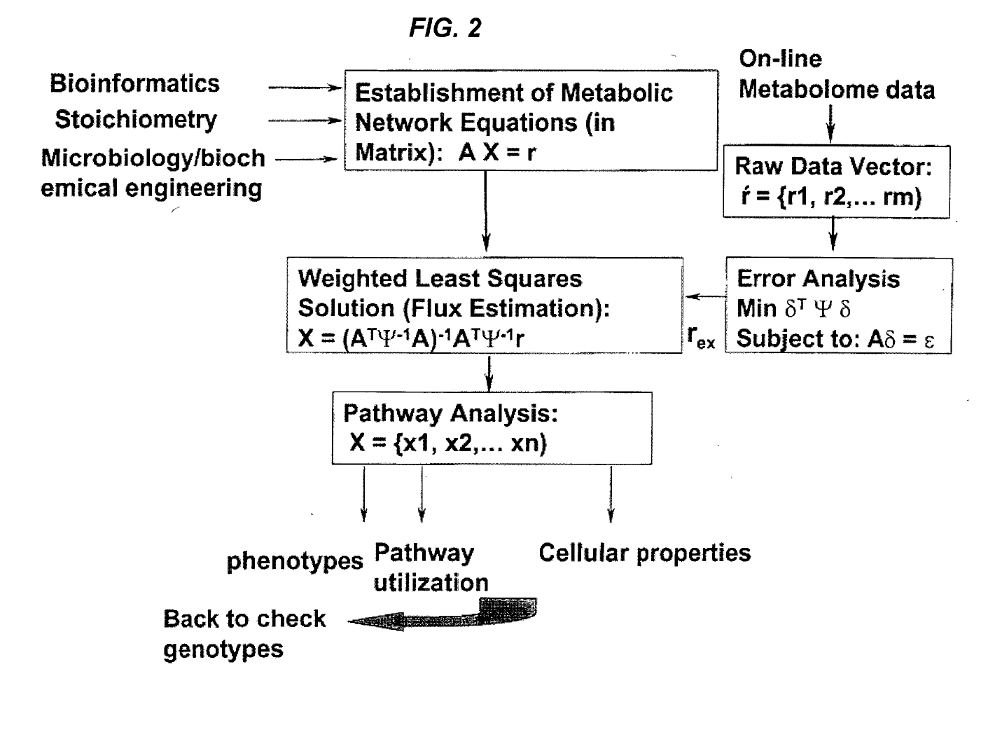

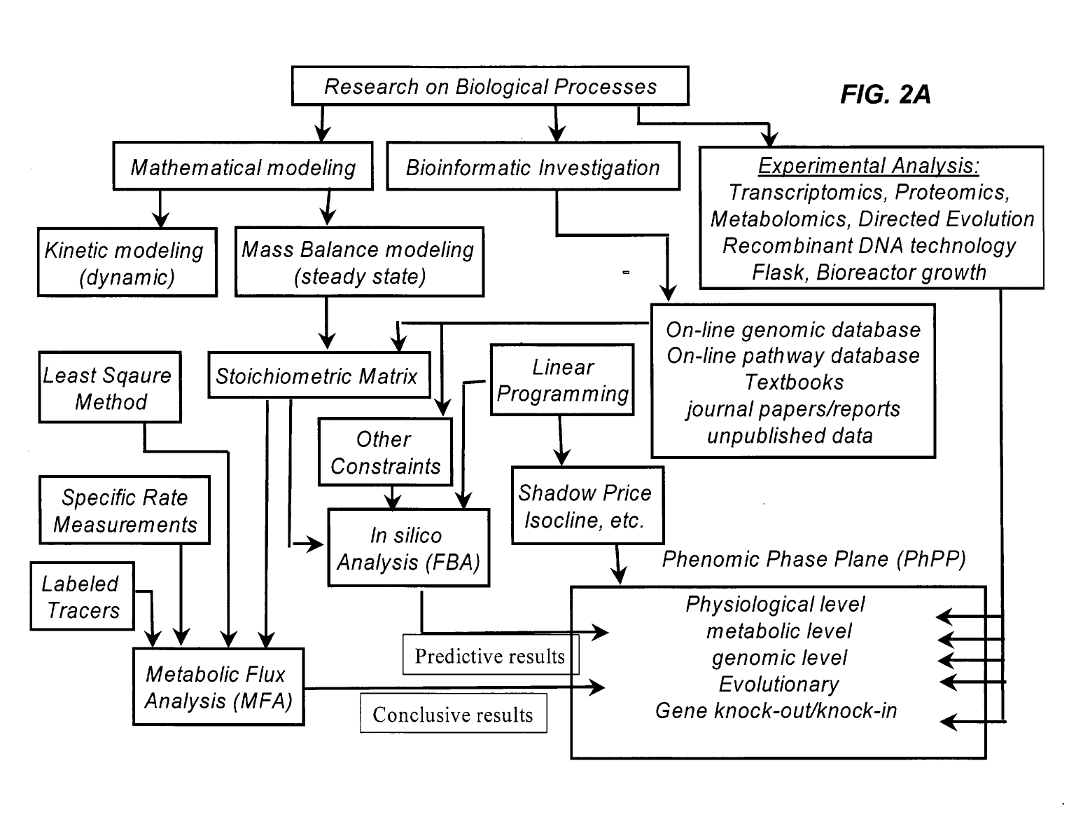

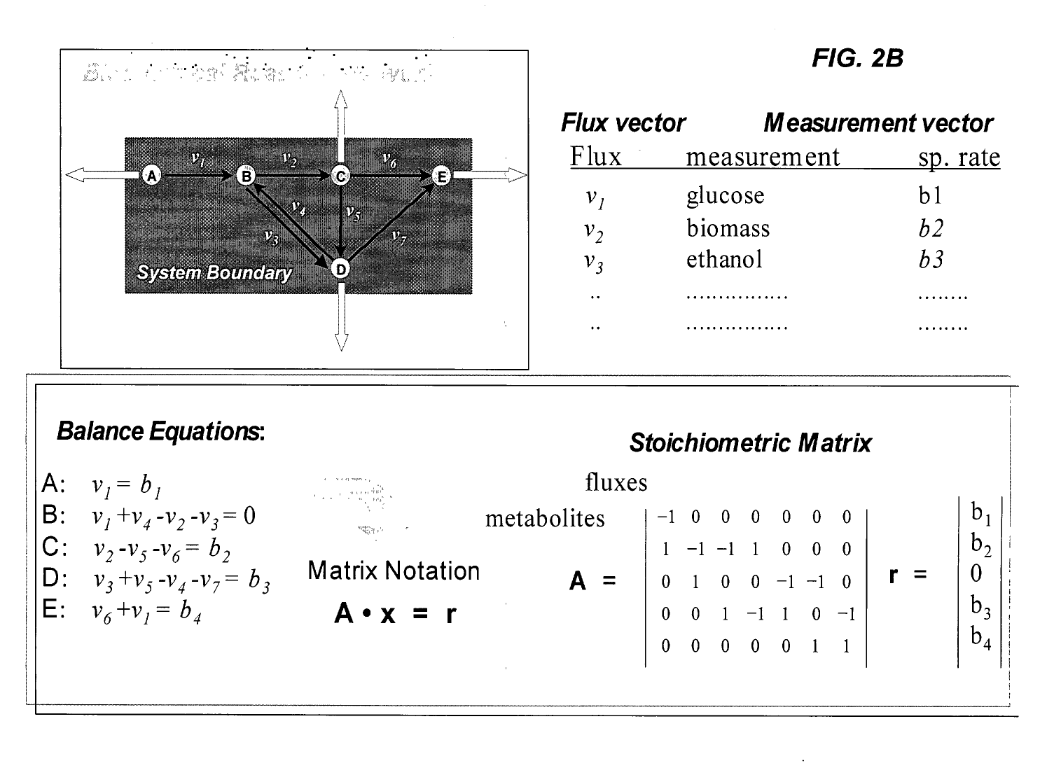

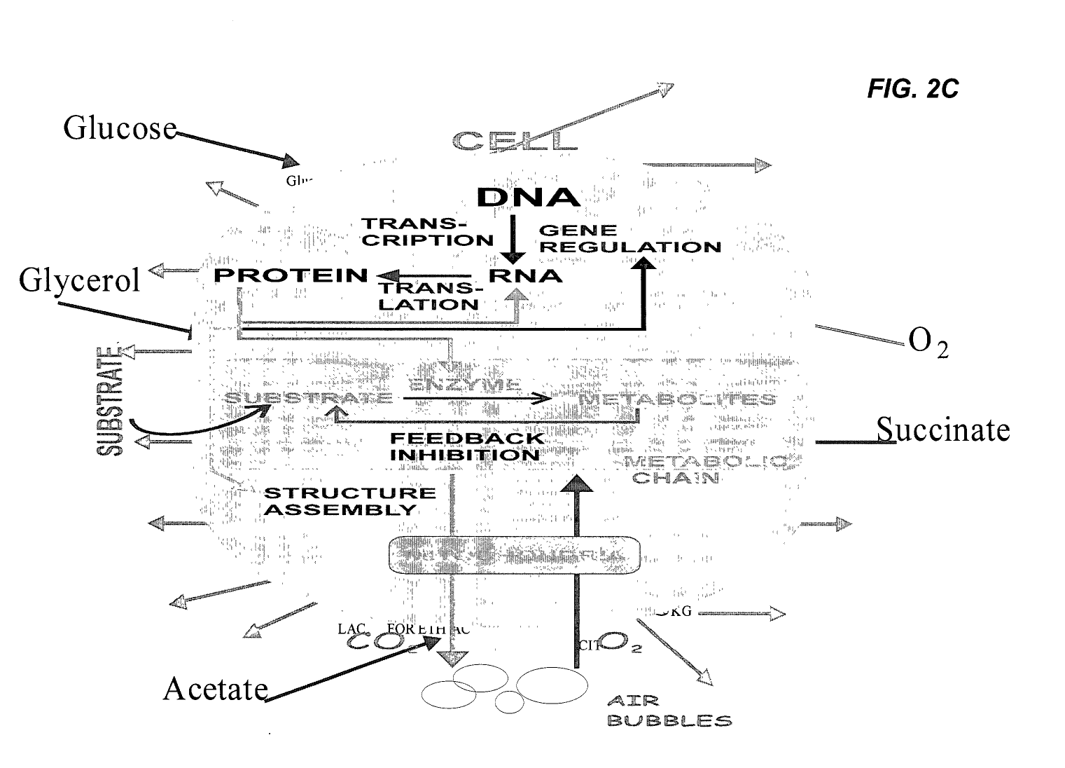

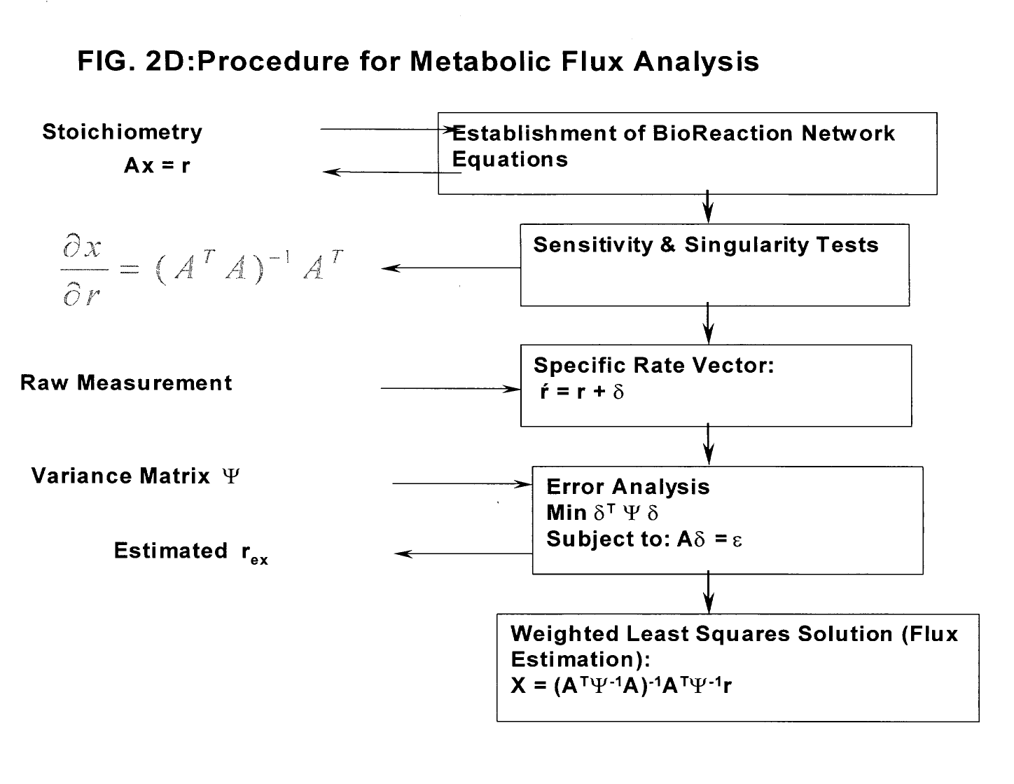

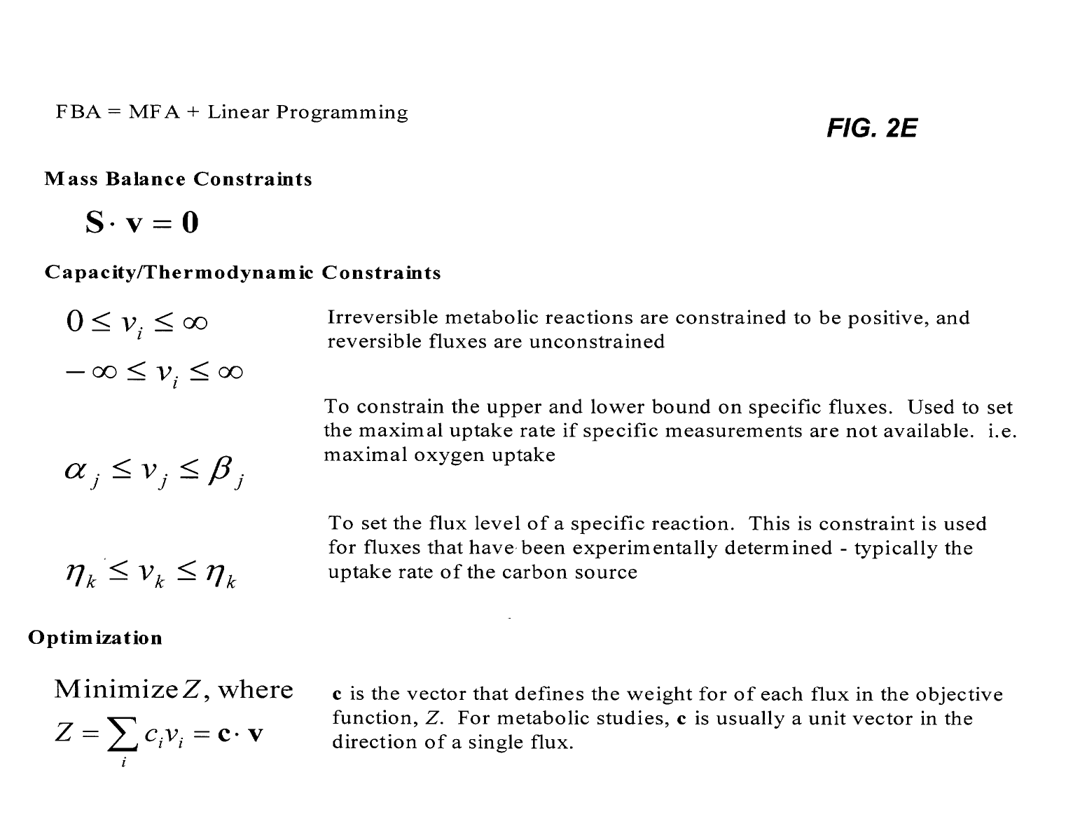

The invention also provides methods for real time monitoring of changes in measured cell and cell culture metabolic parameters over time. In one aspect of the invention, the methods comprise use of a computer-implemented program to real time monitor the change in measured metabolic parameters over time. In one aspect, the methods and programs also comprise the analysis and displaying of the resulting processed data. One exemplary computer-implemented program comprises a computer-implemented method as set forth in FIG. 2. In this and other computer-implemented methods that can be used, this exemplary paradigm comprises use of metabolic network equations, metabolic pathway analyses, error analysis, such as a weighted least squares solution to give a flux estimation and the like.

FIGS. 2A through 2E further show various aspects and examples of the present invention. FIG. 2A shows the overall structure of the system biology frame work within which the present invention may be applied. FIG. 2B illustrates an example of the metabolic network equation of a hypothetical cell to demonstrate underlying physical processes of the equation. FIG. 2C illustrates exemplary application of the metabolic flux analysis. FIG. 2D shows one example of a procedure for the metabolic flux analysis. FIG. 2E provides an example for the constraints in the metabolic flux balance analysis (FBA). These features of the invention are further explained and illustrated throughout the specification of this application.

The computer-implemented method in FIG. 2 may include the following major operations. First, the metabolic network equations for a specified cell are established from known genetic and biochemical properties for that cell. Such properties may be obtained from certain known databases, such as, e.g., a bioinformatics database, a stoichiometry database, a genomics or a proteonomics database, a microbiology database, a biochemical engineering database and the like. These and other databases may be accessed via proper communication links or channels such as various computer networks including the Internet.

The metabolic network equations that may be derived from such information on a particular cell or cell culture may be based on the assumption that the total mass of the transient material in different metabolic fluxes at different node sites of the cell is conserved. When the metabolic fluxes of the cell reach a steady state, the metabolic network equations may be expressed in a linear matrix equation: AX=r, where A is a matrix representing the stoichiometric coefficients or parameters of the given cell or cell culture, X is a vector representing all metabolic fluxes of the cell or cell culture, and r is a vector representing specific rates of measured metabolic parameters. The metabolic parameters, or r, may be measured in real time by various means. Hence, for a given A of the cell, once the specific rates in the vector r are determined from real time measurements or prior measurements, the metabolic fluxes (X) may be determined.

The measured metabolic parameter can comprise an increase or a decrease in a secondary metabolite, such as glucose, glycerol, ethanol or methanol. The measured metabolic parameter can comprise an increase or a decrease in an organic acid, such as acetate, butyrate, succinate, oxaloacetate, fumarate, alpha-ketoglutarate or phosphate. The measured metabolic parameter can comprise an increase or a decrease in intracellular or culture pH. The measured metabolic parameter can comprise an increase or a decrease in input or output of a gas, e.g., oxygen, methanol, and the like.

In one aspect, a computer program is implemented with appropriate computer hardware to perform the computation of X at a high speed so that the computing time is relatively short during which the change in the cell under culturing is small. That is, the processing speed of a full metabolic flux analysis (MFA) is faster than the growth rate of the cell under culturing. In this context, the computer-implemented metabolic flux analysis is deemed to be in real time while the cell culturing is in progress at the same time.

As shown in FIG. 2, this embodiment of the computer-implemented method may also include a process to obtain on-line metabolome data for the data vector r in the matrix equation AX=r. Such data is used to form the raw data vector r. The raw data vector may be further processed through an error analysis process to produce a modified data vector r for the actual MFA computation. The source of the on-line metabolome data may be the on-line sensing subsystem that is coupled to the cell growth environment. In this configuration, the operating speed of the on-line sensing subsystem should be faster that the growth rate of the cell under culturing so that the time for a full measurement by the sensing subsystem and the full MFA computation by the computer is relatively short to be in real time. Alternatively, the source of the on-line metabolome data may also be from an electronic data file or database where prior measurements or metabolome data files for the cell of interest are stored. Different from the on-line MFA for monitoring the metabolic fluxes of cells under culturing, such non real-time metabolome data may be used to predict the metabolic fluxes of a selected cell and thus may be used in the cell selection process or design of the cell culturing conditions.

The computer-implemented MFA computation may be carried out with any one or a combination of various suitable computation techniques to achieve desired processing speed and computation accuracy. One technique for improving the computation accuracy, for example, is to use weighted least square solution as shown in FIG. 2. Upon completion of the computation for X, the metabolic flux pathways in the cell may be analyzed to determine phenotypes, analyze pathway utilization, and investigate certain cellular properties of the cells.

The above computer-implemented MFA may be implemented in proper hardware systems to provide novel cell growth or engineering systems with real-time MFA capability. The following sections describe embodiments of such systems as examples to illustrate this aspect of the invention.

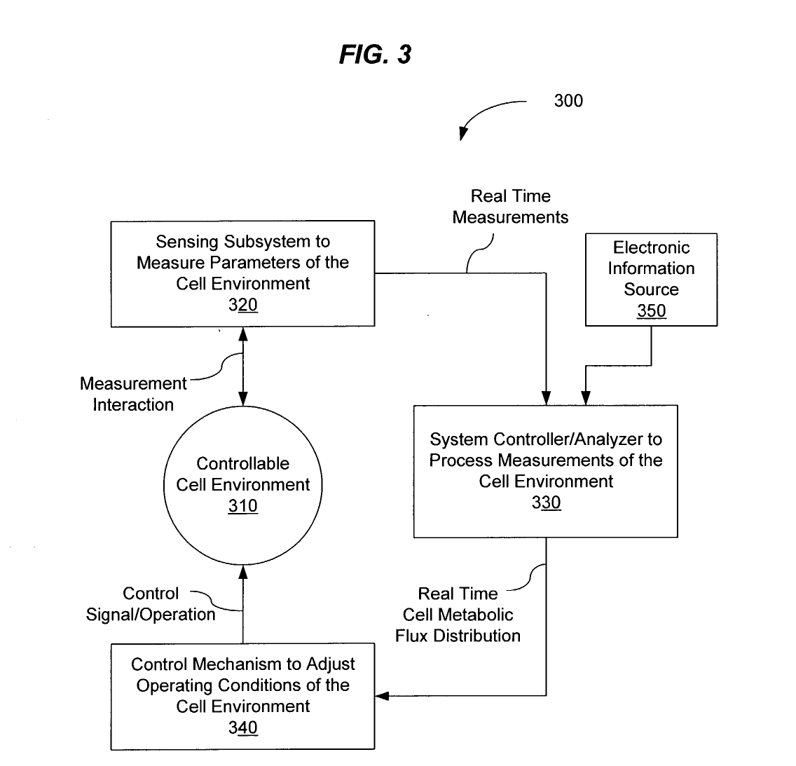

FIG. 3 illustrates one embodiment of a cell growth system 300 with an on-line sensing subsystem 320 for monitoring the cell growth in a controllable cell environment 310 in real time, an on-line data processing mechanism 330 for processing the measurements in real time, and a control mechanism 340 for controlling the conditions of the cell growth where the control may be made in response to the real time measurements. The cell environment 310 may be implemented in various controllable or alterable configurations, examples of which include but are not limited to, a fermentor, a bioreactor, a cell culturing flask, and a cell culturing plate. The sensing subsystem 320 may include one or more sensing devices that are coupled to the cell environment 310 for taking measurements. Examples of sensing devices in the sensing subsystem 320 include but are not limited to, sensing devices of measuring properties of the cells under culturing (e.g., biomass monitor based on optical density measurement), sensing devices for the cell environment (e.g., mass spectrometer for OUR, CER, and RQ measurements), and sensing devices for measuring properties of the metabolites (e.g., on-line bioanalyzer).

The on-line data processing mechanism 330 generally includes a computer which is programmed to retrieve proper genetic and biochemical information from proper sources, carry out the MFA computation, and present graphical or textual display of the MFA results. The computer is electronically interfaced with the devices in the sensing subsystem 320 to receive real-time measurements. Such electronic interface includes analog-to-digital converters (ADCs) to convert the measurements into computer-readable digital data. Such ADCs may be built in the signal output mechanisms of the sensing devices or the sensing subsystem 320, or may be separate units connected between the computer and the sensing subsystem 320. The computer may be linked to other external electronic information source 350 for retrieving certain genetic and biochemical information of various cells of interests and other data needed for the MFA process. Examples for the electronic information source 350 include but are not limited to an electronic storage device, another computer or server, a computer network such as a local area network or a wide area network or the Internet.

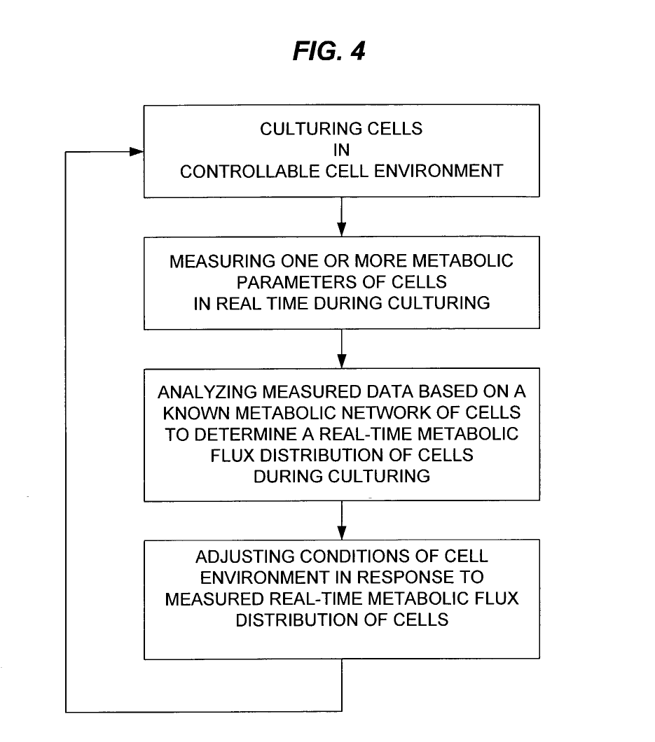

The control mechanism 340 provides input to the cell environment 310 to change the cell culturing conditions (e.g., temperature) or to change the materials in the cell environment 310 (e.g., the pH value). The input may be changed in response to the real time cell metabolic flux distribution (MFD) produced by the system analyzer 330. The control may be carried by a human operator or automatically through electronic and other automated control mechanisms. FIG. 4 shows one exemplary cell engineering process that may be achieved by using the system 300 in FIG. 3.

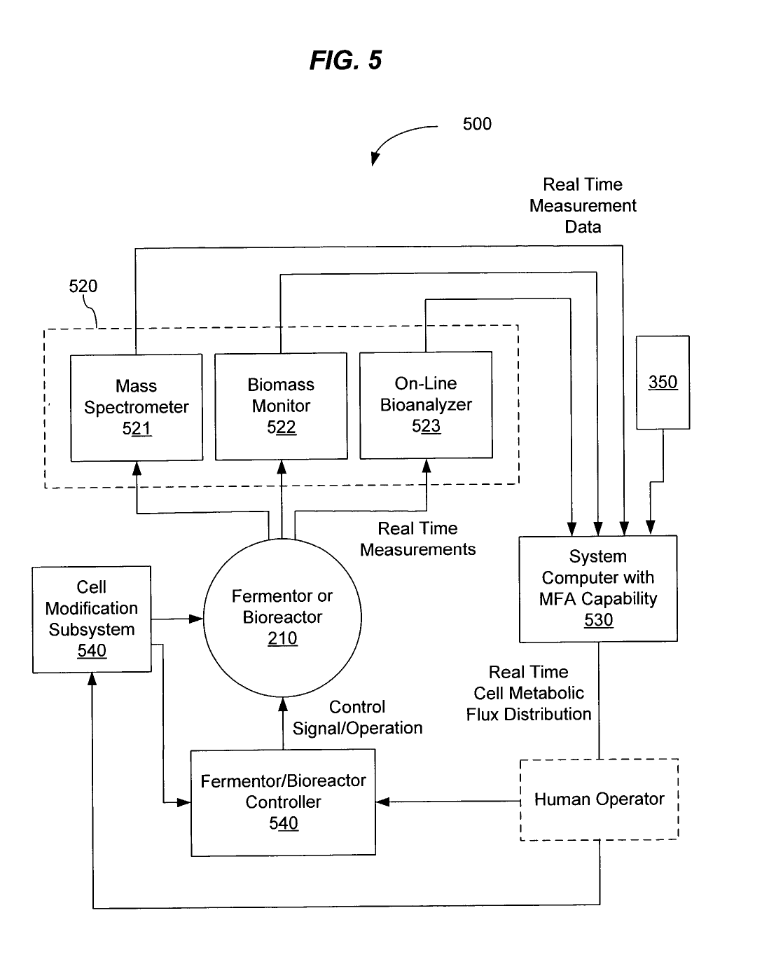

FIG. 5 illustrates one implementation of a cell growth and engineering system 500 in part based on the system 300 shown in FIG. 3, where a cell modification subsystem 540 is used to modify or engineer the cells according to real-time measurements of the cells under culturing in a controllable cell environment 510 such as a fermentor or bioreactor. In this system, the sensing subsystem 520 is shown to include a mass spectrometer, a biomass monitor, and an on-line bioanalyzer that are respectively connected to the system computer 530 for MFA computation. A controller 540 for the fermentor or bioreactor 510 is connected to receive input control signals from both the cell modification subsystem 540 and the system computer 530. The control signals to the controller 540 based on the MFA computation may be automatically fed to the controller 540 via computer-based intelligence or a human operator. The MFA results from the system computer 530 may also be sent to the cell modification subsystem via an electronic interface or a human operator to modify the cells.

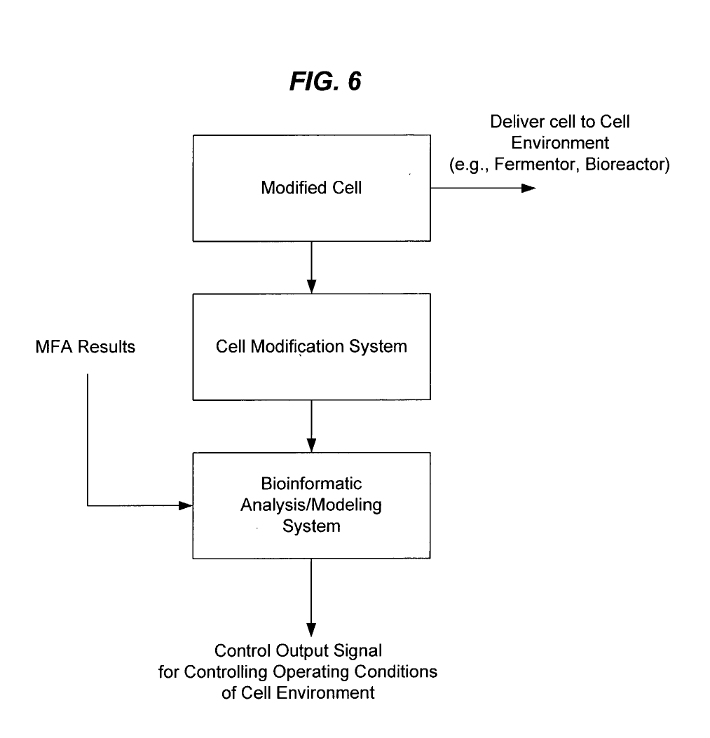

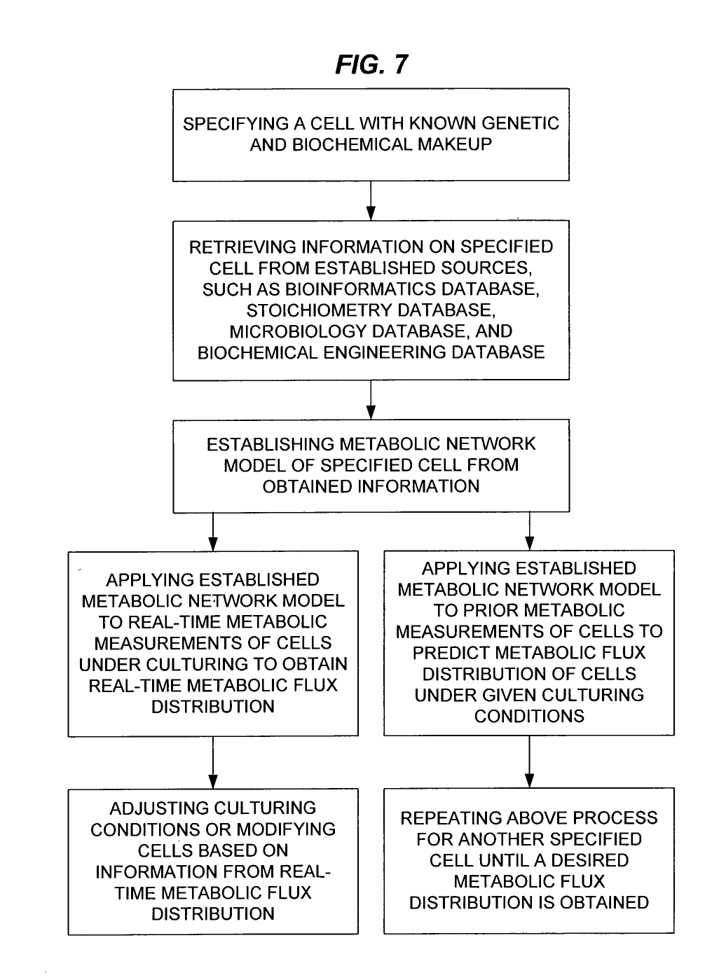

FIG. 6 shows one example of a cell modification subsystem that may be used in the system 500 in FIG. 5. FIG. 7 shows operations that may be carried out using the system 500 in FIG. 5.

Various aspects, features, and implementations of the invention are now described in detail in the following sections.

In one aspect of the invention, a nucleic acid (or, the nucleic acid) responsible for the altered phenotype is identified, re-isolated, again modified (e.g., either stochastically or non-stochastically), reinserted into the cell, and the process of real-time metabolic flux analysis is iteratively repeated. The process can be iteratively repeated until a desired phenotype is engineered. For example, a plant cell and plant cell culture is subjected to iterative repetition of the methods of the invention until a new plant cell is made that comprises a desired new phenotype, e.g., enhanced growth, nutritional value or insect or drought resistance, or all or some of these characteristics. A pathogenic microorganism can be subjected to iterative repetition of the methods of the invention until it becomes non-pathogenic. A microorganism can be engineered to become lethal to another organism, such as an insect, or, to produce a variety of antibiotics or other compositions. Microorganisms can be subjected to iterative repetition of the methods of the invention to engineer, e.g., increased yield of desired products, removal of unwanted co-metabolites, improved utilization of inexpensive carbon and nitrogen sources, and adaptation to fermentor/bioreactor growth conditions, increased production of a primary metabolite, increased production of a secondary metabolite, increased tolerance to acidic conditions, increased tolerance to basic conditions, increased tolerance to organic solvents, increased tolerance to high salt conditions and increased tolerance to high or low temperatures.

A complete biosynthetic pathway can be inserted into a cell. Any cell phenotype can be modified or any phenotype can be added to a cell using the methods of the invention, without limitation. The invention can be practiced in combination with other methods for inserting and screening for metabolic pathways, see, e.g., U.S. Pat. No. 6,268,140, which describes producing and screening combinatorial metabolic libraries of multimeric proteins, or, U.S. Pat. No. 5,712,146, which describes vectors encoding polyketide synthases which in turn catalyze the production of a variety of polyketides.

Definitions

Unless defined otherwise, all technical and scientific terms used herein have the meaning commonly understood by a person skilled in the art to which this invention belongs. As used herein, the following terms have the meanings ascribed to them unless specified otherwise.

The terms “array” or “microarray” or “biochip” or “chip” as used herein is a plurality of target elements, each target element comprising a defined amount of one or more polypeptides or nucleic acids immobilized onto a defined area of a substrate surface, as discussed in further detail, below.

As used herein, the terms “computer” and “processor” are used in their broadest general contexts and incorporate all such devices, as described in detail, below.

The term “saturation mutagenesis” or “GSSM” includes a method that uses degenerate oligonucleotide primers to introduce point mutations into a polynucleotide, as described in detail, below.

The term “optimized directed evolution system” or “optimized directed evolution” includes a method for reassembling fragments of related nucleic acid sequences, e.g., related genes, and explained in detail, below.

The term “synthetic ligation reassembly” or “SLR” includes a method of ligating oligonucleotide fragments in a non-stochastic fashion, and explained in detail, below.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-73; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The terms “cell,” “cells” or “cell culture” for growth in a controllable cell environment are used in their broadest sense and include all self-replicatory biological systems, including plasmids, prions, phage, virions (e.g., DNA and RNA viruses) and the like. The term includes all cells, including all prokaryotic, eukaryotic and archaeal cells e.g., bacterial cells, insect cells, plant cells, yeast cells and mammalian cells. The methods and compositions (e.g., systems, programs) of the invention can be used to determine real time MFA, and optimal culture conditions, for all of these self-replicatory biological systems.

Generating and Manipulating Nucleic Acids

The methods of the invention include modifying the genetic composition of a cell by addition of a heterologous nucleic acid into the cell or modification of a homologous gene in the cell. Nucleic acids can be isolated from a cell, recombinantly generated or made synthetically. The sequences can be isolated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

Amplification of Nucleic Acids

In practicing the methods of the invention, nucleic acids encoding heterologous or homologous, or modified nucleic acids, can be reproduced by, e.g., amplification. Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell-or a cDNA library are amplified. The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Modification of Nucleic Acids

In practicing the methods of the invention, the genetic composition of a cell is altered by, e.g., modification of a homologous gene ex vivo, followed by its reinsertion into the cell. A homologous, heterologous or gene selected by the methods of the invention can be altered by any means, including, e.g., random or stochastic methods, or, non-stochastic, or “directed evolution,” methods.

Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Techniques in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793.

Non-stochastic, or “directed evolution,” methods include, e.g., saturation mutagenesis (GSSM), synthetic ligation reassembly (SLR), or a combination thereof. In one aspect of the invention, nucleic acids are selected, using real-time metabolic flux analysis, for conferring a new or modified phenotype on a cell, isolated, modified and reinserted into a cell to reiterate the steps of the methods of the invention. Polypeptides encoded by isolated and/or modified nucleic acids can be screened for an activity before their reinsertion into the cell by, e.g., using a capillary array platform. See, e.g., U.S. Pat. Nos. 6,280,926; 5,939,250.

Saturation Mutagenesis, or, GSSM

In one aspect of the invention, non-stochastic gene modification, a “directed evolution process,” can be used to modify a gene to be inserted into a cell to add or modify a phenotype. Variations of this method have been termed “gene site-saturation mutagenesis,” “site-saturation mutagenesis,” “saturation mutagenesis” or simply “GSSM.” It can be used in combination with other mutagenization processes. See, e.g., U.S. Pat. Nos. 6,171,820; 6,238,884. In one aspect, GSSM comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids.

The N,N,G/T cassette is used for illustrative (not limiting) purposes in this invention; thus, it is appreciated that in addition to an N,N,G/T cassette, other cassettes, such as a 32-fold degenerate N,N,G/C cassette or a 48-fold degenerate N,N,C/G/T or a 48-fold degenerate N,N,A,C/G cassette can also be used to introduce the full range of all 20 acids at a given codon position; and this invention specifically provides that these cassettes can also be used instead of an N,N,G/T in alternative aspects of this invention. Furthermore, this invention provides that all degenerate as well as non-degenerate cassettes can be used to alter a polynucleotide sequence (whether in a coding region or a non-coding region); for example in the case of a coding region the ration of codons to amino acids encoded can be 1:1 as well as in excess of 1:1. Thus if the ratio of codon degeneracy:number of encoded amino acids is exactly 1:1, then a 19-fold degenerate cassette can be used to introduce all 19 possible changes to a codon position.

In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used - either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position×100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased affinity or avidity to an antigen), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

Another non-stochastic gene modification, a “directed evolution process,” that can be can be used in the methods of the invention to modify a gene to be inserted into a cell to add or modify a phenotype has been termed “synthetic ligation reassembly,” or simply “SLR.” SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. (U.S. Ser. No.) 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10¹⁰⁰ different chimeras. SLR can be used to generate libraries comprised of over 10¹⁰⁰⁰ different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotide molecules. These parental oligonucleotide templates thus serve as a source of sequence information that aids in the design of the nucleic acid building blocks that are to be mutagenized, e.g., chimerized or shuffled.

In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another embodiment, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups.

Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design.

The saturation mutagenesis and optimized directed evolution methods also can be used to generate these amounts of different progeny molecular species.

It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecularly homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutageneis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

Thus, according to another aspect, a nucleic acid building block can be used to introduce an intron. Thus, functional introns may be introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

In practicing the methods of the invention, nucleic acids can also be modified by a method comprising an optimized directed evolution system. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found in U.S. Ser. No. 09/332,835. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a 1/3 chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

These methods are directed to the use of repeated cycles of reductive reassortment, recombination and selection that allow for the directed molecular evolution of a nucleic acid encoding an polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a 1/3 chance (assuming 3 parents) that a oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental Variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Determining Crossover Events

Embodiments of the invention include a system and software that receive a desired crossover probability density function (PDF), the number of parent genes to be reassembled, and the number of fragments in the reassembly as inputs. The output of this program is a “fragment PDF” that can be used to determine a recipe for producing reassembled genes, and the estimated crossover PDF of those genes. The processing described herein can be performed in MATLAB® (The Mathworks, Natick, Mass.) a programming language and development environment for technical computing.

Iterative Processes

In practicing the methods of the invention, the process can be iteratively repeated. For example a nucleic acid (e.g., a message, a gene, an operon and/or a partial or a complete biosynthetic pathway) responsible for an altered phenotype is identified, re-isolated, again modified, reinserted into the cell, and the process of real-time metabolic flux analysis is iteratively repeated. The process can be iteratively repeated until a desired phenotype is engineered. For example, an entire biochemical pathway can be engineered into a cell. Any cell phenotype can be modified or any phenotype can be added to a cell using the methods of the invention, without limitation.

Nucleic acids can be modified using either stochastic or non-stochastic methods. In various aspects, the methods generate sets of chimeric nucleic acid and protein molecules, followed by insertion into a cell, culturing, and then screening by using real-time metabolic flux analysis for a particular activity, such as a changed or added desired phenotype. The invention is not limited to only a single round of screening. Based on this determination, a second round of reassembly can take place that enriches for progeny having a desired property or incurring a desired phenotype.

Similarly, if it is determined that a particular oligonucleotide has no affect at all on the desired trait (e.g., a new phenotype), it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

Automated Control of Reactions

The process of generating any of the reactions of the methods of the invention can be automated with the assistance of automated devices and robotic instruments. For example, in one aspect, a cell growth monitor device is used for real-time metabolic flux analysis, such as a Wedgewood Technology, Inc., Cell Growth Monitor model 652. As noted below, this device can be linked to a computer system. Another exemplary device is a TECAN GENESIS™ programmable robot made by Tecan Corporation (Hombrechtikon, Switzerland), which can be interfaced with a computer that determines the quantities of each oligonueleotide fragment to yield a resulting PDF. By linking a computer system that determines the proper quantities of each oligonucleotide to an automated robot, a complete ligation reassembly system is produced. Data links through serial or other interfaces will allow the data files generated from the ligation reassembly calculations to be forwarded in the proper format for the robotic system to automatically begin allocating the proper quantities of each oligonucleotide fragment into a reaction tube.

The automated system can include a plurality of oligonucleotide fragments derived from a series of nucleic acid sequence variants, wherein said fragments are configured to join one another at unique overhangs. The system also has a data input field configured to store a target number of crossover events in for each of the variant sequences. Within the system is also a prediction module configured to determine the quantity of each of the fragments to admix together so that mixing the fragments results in a population of progeny molecules that are enriched for crossover events corresponding to the target number. The system also provides a robotic arm linked to the prediction module through a communication interface for automatically mixing the fragments in the determined quantities.

Mutagenized Oligonucleotides

While the optimized directed evolution method can use oligonucleotides that have a 100% fidelity to their parent polynucleotide sequence, this level of fidelity is not required. For example, if a set of three related parental polynucleotides are chosen to undergo ligation reassembly in order to create, e.g., a new phenotype, a set of oligonucleotides having unique overlapping regions can be synthesized by conventional methods. However a set of mutagenized oligonucleotides could also be synthesized. These mutagenized oligonucleotides are preferably designed to encode silent, conservative, or non-conservative amino acids.

The choice to enter a silent mutation might be made to, for example, add a region of nucleotide homology two fragments, but not affect the final translated protein. A non-conservative or conservative substitution is made to determine how such a change alters the function of the resultant polypeptide. This can be done if, for example, it is determined that mutations in one particular oligonucleotide fragment were responsible for increasing the activity of a peptide. By synthesizing mutagenized oligonucleotides (e.g.: those having a different nucleotide sequence than their parent), one can explore, in a controlled manner, how resulting modifications to the peptide or protein sequence affect the activity of the peptide or polypeptide.

Another method for creating variants of a nucleic acid sequence using mutagenized fragments includes first aligning a plurality of nucleic acid sequences to determine demarcation sites within the variants that are conserved in a majority of said variants, but not conserved in all of said variants. A set of first sequence fragments of the conserved nucleic acid sequences are then generated, wherein the fragments bind to one another at the demarcation sites. A second set of fragments of the not conserved nucleic acid sequences are then generated by, for example, a nucleic acid synthesizer. However, the not conserved, sequences are generated to have mutations at their demarcation site so that the second fragments have the same nucleotide sequence at the demarcation sites as said first fragments. This allows the not conserved sequences to still hybridize during the ligation reaction to the other parental sequences. Once the fragments are generated, a desired number of crossover events can be selected for each of the variants. The quantity of each of the first and second fragments is then calculated so that a ligation/incubation reaction between the calculated quantities of the first and second fragments will result in progeny molecules having the desired number of crossover events. In Silico, or Computer, Models

In silico, or computer program-implemented, paradigms can be used in practicing the methods of the invention to design altered or new nucleic acids to modify cells for the creation of new phenotypes. The invention also provides articles comprising machine-readable medium including machine-executable instructions and systems, e.g., computer systems, to practice these in silico, or computer program-implemented methods of the invention.

One exemplary in silico method that can be used in practicing the methods of the invention for generating man-made polynucleotide sequences for the creation of new phenotypes detects shared domains between a plurality of template polynucleotides. It does so by aligning the template polynucleotides and identifying all sequence strings having a certain percentage of homology, e.g., about 75% to 95% sequence identity, that are shared between all of the template polyiiucleotides. This detects shared domains between the template polynucleotides. Next, domain sequences are switched from one template polynucleotide with the sequence of a corresponding domain. This is repeated until all domains have been switched with a corresponding domain on another template polynucleotide, thereby generating in silico a library of man-made polynucleotide sequences from a set of template polynucleotides.

In silico, or computer program-implemented, methods can also be used in practicing the methods of the invention to analyze metabolic flux data; see, e.g., Covert (2001) Trends Biochem. Sci. 26(3):179-186; Jamshidi (2001) Bioinformatics 17(3):286-287. For example, the quantitative relationship between a primary carbon source (e.g., for bacteria, acetate or succinate) uptake rate, oxygen uptake rate, and maximal cellular growth rate can be modeled in silico, and used complementary to the “real-time” or “on-line” monitoring of the invention, see, e.g., Edwards (2001) Nat. Biotechnol. 19(2):125-130. The effects of gene deletions in a central metabolic pathway can also be modeled in silico, and used complementary to the “real-time” or “on-line” monitoring of the invention, see, e.g., Edwards (2000) Proc. Natl. Acad. Sci. USA 97(10):5528-5533.

Measuring Metabolic Parameters

The methods of the invention involve whole cell evolution, or whole cell engineering, of a cell to develop a new cell strain having a new phenotype. To detect the new phenotype, at least one metabolic parameter of a modified cell is monitored in the cell in a “real time” or “on-line” time frame. In one aspect, a plurality of cells, such as a cell culture, is monitored in “real time” or “on-line.” In one aspect, a plurality of metabolic parameters is monitored in “real time” or “on-line.”

Metabolic flux analysis (MFA) is based on a known biochemistry framework. A linearly independent metabolic matrix is constructed based on the law of mass conservation and on the pseudo-steady state hypothesis (PSSH) on the intracellular metabolites. In practicing the methods of the invention, metabolic networks are established, including:

identity of all pathway substrates, products and intermediary metabolites identity of all the chemical reactions interconverting the pathway metabolites, the stoichiometry of the pathway reactions,

identity of all the enzymes catalyzing the reactions, the enzyme reaction kinetics,

the regulatory interactions between pathway components, e.g. allosteric interactions, enzyme-enzyme interactions etc,

intracellular compartmentalization of enzymes or any other supramolecular organization of the enzymes, and,

the presence of any concentration gradients of metabolites, enzymes or effector molecules or diffusion barriers to their movement.

Once the metabolic network for a given strain is built, mathematic presentation by matrix notion can be introduced to estimate the intracellular metabolic fluxes if the on-line metabolome data is available.

Metabolic phenotype relies on the changes of the whole metabolic network within a cell. Metabolic phenotype relies on the change of pathway utilization with respect to environmental conditions, genetic regulation, developmental state and the genotype, etc. In one aspect of the methods of the invention, after the on-line MFA calculation, the dynamic behavior of the cells, their phenotype and other properties are analyzed by investigating the pathway utilization. For example, if the glucose supply is increased and the oxygen decreased during the yeast fermentation, the utilization of respiratory pathways will be reduced and/or stopped, and the utilization of the fermentative pathways will dominate. Control of physiological state of cell cultures will become possible after the pathway analysis. The methods of the invention can help determine how to manipulate the fermentation by determining how to change the substrate supply, temperature, use of inducers, etc. to control the physiological state of cells to move along desirable direction. In practicing the methods of the invention, the MFA results can also be compared with transcriptome and proteome data to design experiments and protocols for metabolic engineering or gene shuffling, etc.

In practicing the methods of the invention, any modified or new phenotype can be conferred and detected, including new or improved characteristics in the cell. Any aspect of metabolism or growth can be monitored.

Monitoring Expression of an mRNA Transcript

In one aspect of the invention, the engineered phenotype comprises increasing or decreasing the expression of an mRNA transcript or generating new transcripts in a cell. mRNA transcript, or message can be detected and quantified by any method known in the art, including, e.g., Northern blots, quantitative amplification reactions, hybridization to arrays, and the like. Quantitative amplification reactions include, e.g., quantitative PCR, including, e.g., quantitative reverse transcription polymerase chain reaction, or RT-PCR; quantitative real time RT-PCR, or “real-time kinetic RT-PCR” (see, e.g., Kreuzer (2001) Br. J. Haematol. 114:313-318; Xia (2001) Transplantation 72:907-914).

In one aspect of the invention, the engineered phenotype is generated by knocking out expression of a homologous gene. The gene's coding sequence or one or more transcriptional control elements can be knocked out, e.g., promoters, enhancers and the like. Thus, the expression of a transcript can be completely ablated or only decreased.

In one aspect of the invention, the engineered phenotype comprises increasing the expression of a homologous gene. This can be effected by knocking out of a negative control element, including a transcriptional regulatory element acting in cis- or trans-, or, mutagenizing a positive control element.

As discussed below in detail, one or more, or, all the transcripts of a cell can be measured by hybridization of a sample comprising transcripts of the cell, or, nucleic acids representative of or complementary to transcripts of a cell, by hybridization to immobilized nucleic acids on an array.

Monitoring Expression of a Polypeptides, Peptides and Amino Acids In one aspect of the invention, the engineered phenotype comprises increasing or decreasing the expression of a polypeptide or generating new polypeptides in a cell. Polypeptides, peptides and amino acids can be detected and quantified by any method known in the art, including, e.g., nuclear magnetic resonance (NMR), spectrophotometry, radiography (protein radiolabeling), electrophoresis, capillary electrophoresis, high performance liquid chromatography (HPLC), thin layer chromatography (TLC), hyperdiffusion chromatography, various immunological methods, e.g. immunoprecipitation, immunodiffusion, immuno-electrophoresis, radioimmunoassays (RIAs), enzyme-linked immunosorbent assays (ELISAs), immuno-fluorescent assays, gel electrophoresis (e.g., SDS-PAGE), staining with antibodies, fluorescent activated cell sorter (FACS), pyrolysis mass spectrometry, Fourier-Transform Infrared Spectrometry, Raman spectrometry, GC-MS, and LC-Electrospray and cap-LC-tandem-electrospray mass spectrometries, and the like. Novel bioactivities can also be screened using methods, or variations thereof, described in U.S. Pat. No. 6,057,103. Furthermore, as discussed below in detail, one or more, or, all the polypeptides of a cell can be measured using a protein array.

Biosynthetically directed fractional ¹³C labeling of proteinogenic amino acids can be monitored by feeding a mixture of uniformly ¹³C-labeled and unlabeled carbon source compounds into a bioreaction network. Analysis of the resulting labeling pattern enables both a comprehensive characterization of the network topology and the determination of metabolic flux ratios of the amino acids; see, e.g., Szyperski (1999) Metab. Eng. 1:189-197.

Monitoring the Expression of a Metabolites and Biosynthetic Pathways

In one aspect, primary and secondary metabolites are the measured metabolic parameters. Any relevant primary and secondary metabolite can be monitored in real time. For example, the measured metabolic parameter can comprise an increase or a decrease in a primary or a secondary metabolite. A metabolite can be, e.g., glucose, glycerol, methanol and the like. The measured metabolic parameter can comprise an increase or a decrease in an organic acid, such as acetate, butyrate, succinate, oxaloacetate, fumarate, alpha-ketoglutarate or phosphate and the like. In one aspect, the metabolic parameter measured comprises an increase or a decrease in a gas, e.g., oxygen, methanol, hydrogen and the like.

The choice of which metabolite or metabolic or biosynthetic pathway to monitor “on-line” or in “real time” depends on which phenotype is desired to be added or modified. For example, limonene and other downstream metabolites of geranyl pyrophosphate can be monitored “on-line” or in “real time” as in U.S. Pat. No. 6,291,745, which monitored to generate means for insect control in plants, see, e.g.,. Metabolites/antibiotics in the supernatant in Bacillus subtilis can be monitored for effective insecticidal, antifungal and antibacterial agents, see, e.g., U.S. Pat. No. 6,291,426. The methods of the invention can also be used to monitor metabolites of the tricarboxylic acid cycle and glycolysis, as in a Bacillus subtilis strain by Sauer (1997) Nat. Biotechnol. 15:448-452 (who also used fractional ¹³C-labeling and two-dimensional nuclear magnetic resonance spectroscopy). The penicillin biosynthetic pathway can be monitored in real time in, e.g., Penicillium chrysogenum; see, e.g., Nielsen (1995) Biotechnol. Prog. 11(3):299-305; Jorgensen (1995) Appl. Microbiol. Biotechnol. 43(1): 123-130. Asparagine linked (N-linked) glycosylation can be studied in real time; see, e.g., Nyberg (1999) Biotechnol. Bioeng. 62(3):336-347. The amount of amino acids liberated from peptides in cell cultures grown in a hydrolysate-supplemented medium can be studied in real time; see, e.g., Nyberg (1999) Biotechnol. Bioeng. 62(3):324-335, who studies pathway fluxes in Chinese hamster ovary cells grown in a complex (hydrolysate containing) medium. The methods of the invention can also be used to monitor flux distributions for maximal ATP production in mitochondria, including ATP yields for glucose, lactate, and palmitate; see, e.g., Ramakrishna (2001) Am. J. Physiol. Regul. Integr. Comp. Physiol. 280(3):R695-704. In bacteria, the methods of the invention can also be used to monitor seven essential reactions in the central metabolic pathways, glycolysis, pentose phosphate pathway, tricarboxylic acid cycle, for the growth in a glucose medium, e.g., glucose minimal media. For gene modification, the seven genes encoding these enzymes can be grouped into three categories: (1) pentose phosphate pathway genes, (2) three-carbon glycolytic genes, and (3) tricarboxylic acid cycle genes. See, e.g., Edwards (2000) Biotechnol. Prog. 16(6):927-939.

Monitoring Intracellular pH

In one aspect, the increase or a decrease in intracellular pH is measured “on-line” or in “real time.” The change in intracellular pH can be measured by intracellular application of a dye. The change in fluorescence of the dye can be measured over time.

Any system can be used to determine intracellular pH. If a dye if used, in one exemplary method, whole-field time-domain fluorescence lifetime imaging (FLIM) can be used. FLIM can be used for the quantitative imaging of concentration ratios of mixed fluorophores and quantitative imaging of perturbations to fluorophore environment; in FLIM, the image contrast is derived from the fluorescence lifetime at each point in a two-dimensional image (see, e.g., Cole (2001) J. Microsc. 203(Pt 3):246-257). Near-field scanning optical microscopy (NSOM) is a high-resolution scanning probe technique that can be used to obtain simultaneous optical and topographic images with spatial resolution of tens of nanometers (see, e.g., Kwak (2001) Anal. Chem. 73(14):3257-3262). A frequency domain fluorescence lifetime imaging microscope (FLIM) enables the measurement and reconstruction of three-dimensional nanosecond fluorescence lifetime images (see, e.g., Squire (1999) J. Microsc. 193(Pt 1):36-49).

Monitoring Expression of Gases

In one aspect, the measured metabolic parameter comprises gas exchange rate measurements. Any gas can be monitored, e.g., oxygen, carbon monoxide, carbon dioxide, nitrogen and the like. See, e.g., Follstad (1999) Biotechnol. Bioeng. 63(6):675-683.

Screening Methodologies and “On-line” Monitoring Devices

In practicing the methods of the invention, “real time” or “on-line” cell monitoring devices are used to identify an engineered phenotype in the cell using real-time metabolic flux analysis. Any screening method can be used in conjunction with these “real time” or “on-line” cell monitoring devices.

Cell Growth Monitor Devices

In one aspect, real time monitoring of a plurality of metabolic parameters is done with use of a cell growth monitor device. One exemplary such device is a Wedgewood Technology, Inc. (San Carlos, Calif.), Cell Growth Monitor model 652, which can “real time” or “on-line” monitor a variety of metabolic parameters, including: the uptake of substrates, such as glucose; the levels of intracellular intermediates, such as organic acids, e.g., acetate, butyrate, succinate, oxaloacetate, fumarate, alpha-ketoglutarate and/or phosphate; and, levels of amino acids. Any cell growth monitor device can be used, and these devices can be modified to measure any set of parameters, without limitation. Cell growth monitor device can be used in conjunction with any other measuring or monitoring devices, such as There are some rapid analysis of metabolites at the whole-cell level, using methods such as pyrolysis mass spectrometry, Fourier-Transform Infrared Spectrometry, Raman spectrometry, GC-MS, and LC-Electrospray and cap-LC-tandem-electrospray mass spectrometries.

Capillary Arrays

In addition to “biochip” arrays (see below), capillary arrays, such as the GIGAMATRIX™, Diversa Corporation, San Diego, Calif., can be used to screen for or monitor a variety of compositions, including polypeptides, nucleic acids, metabolites, by-products, antibiotics, metals, and the like, without limitation. Capillary arrays provide another system for holding and screening samples. For example, a sample screening apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample.

A polypeptide or nucleic acid, e.g., a ligand, can be introduced into a first component into at least a portion of a capillary of a capillary array. Each capillary of the capillary array can comprise at least one wall defining a lumen for retaining the first component, and introducing an air bubble into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a microtiter plate having about 100,000 or more individual capillaries bound together.

Arrays, or “BioChips”

In one aspect of the invention, the monitored parameter is transcript expression. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Arrays comprising genomic nucleic acid can also be used to determine the genotype of a newly engineered strain made by the methods of the invention. “Polypeptide arrays” can also be used to simultaneously quantify a plurality of proteins.

The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts.

In practicing the methods of the invention, known arrays and methods of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765. The present invention can use any known array, e.g., GeneChips™, Affymetrix, Santa Clara, Calif.; SpectralChip™ Human BAC Arrays, Spectral Genomics, Houston, Tex.; and their accompanying manufacturer's instructions.

Antibodies and Immunoblots

In practicing the methods of the invention, antibodies can be used to isolate, identify or quantify particular polypeptides or polysaccharides. The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Devices to Monitor Organic Acids and Amino Acids

On-line devices that can monitor organic acids and amino acids can also be used in practicing the methods of the invention. For example, in one aspect, the BIO+ ON-LINE™ (Lachat Instruments, Milwaukee, Wis.) provides near-real-time monitoring of fermentation and mammalian cell culture processes. This device can provide critical information to maximize product yields. Mounted on a cart, this device can be rolled up to a fermentation bank and connected via a stream selector valve. From there, chemical constituent monitoring occurs automatically for ammonia, glucose, glutamate, glutamine, glycerol, lactate and phosphate individually and organic acids as a profile employing ion exclusion chromatography. The BIO+ ON-LINE™ is an integrated sampling system that provides a real solution to this challenging problem using a pumping system combined with a FLOWNAMICS® filter probe which exhibits the following benefits: sterilizable in-place; risk-free sampling due to elimination of bypass filters which recirculate material back into the vessel; sterile, cell-free sampling; accommodates all vessel sizes; minimum dead volume to ensure consistent and accurate sampling and to reduce flush time; durable design and construction to withstand temperatures, pressures, viscosities, shear forces and chemical constituents typical of bioprocess environments.

The BIO+ ON-LINE™ can determine up to four analytes simultaneously using flow injection analysis. The reaction modules can be removed and substituted with other modules. Thus, the user can customize the unit for different fermentation/bioprocess requirements. Additionally, the Ion Chromatography channel can be customized to meet other Liquid Chromatography (LC) needs. While conductivity detection is the default detector, users can connect UV, RI, or other detectors and their own columns to the unit to meet their customized LC separation needs. This system, or variations thereof, is applicable to aerobic and anaerobic bacterial cultures as well as yeast, fungi, algae, insect and mammalian cell cultures.

Other related devices that can be used to practice the invention include the QUIKCHEM® 8000 (Lachat Instruments, Milwaukee, Wis.) which allows high sample throughput coupled with simple and rapid method changeover to maximize productivity in determining ionic species in a diversity of sample matrices from sub-ppb to percent concentrations.

Sources of Cells and Culturing of Cells

The invention provides a method for whole cell engineering of new phenotypes by using real-time metabolic flux analysis. Any cell can be engineered, including, e.g., bacterial, Archaebacteria, mammalian, yeast, fungi, insect or plant cell. In one aspect of the methods of the invention, a cell is modified by addition of a heterologous nucleic acid into the cell. The heterologous nucleic acid can be isolated, cloned or reproduced from a nucleic acid from any source, including any bacterial, mammalian, yeast, insect or plant cell.

In one aspect, the cell can be from a tissue or fluid taken from an individual, e.g., a patient. The cell can be homologous, e.g., a human cell taken from a patient, or, heterologous, e.g., a bacterial or yeast cell taken from the gastrointestinal tract of an individual. The cell can be from, e.g., lymphatic or lymph node samples, serum, blood, chord blood, CSF or bone marrow aspirations, fecal samples, saliva, tears, tissue and surgical biopsies, needle-or punch biopsies, and the like.

Any apparatus to grow or maintain cells can be used, e.g., a bioreactor or a fermentor, see, e.g., U.S. Pat. Nos. 6,242,248; 6,228,607; 6,218,182; 6,174,720; 6,168,949; 6,133,022; 6,133,021; 6,048,721; 5,660,977; 5,075,234.

Real-Time Metabolic Flux Analysis

In the methods of the invention, at least one metabolic parameter of the cell is monitored in real time, i.e., by real time, or “on-line,” flux analysis. In alternative aspects, many parameters of the cells in culture are monitored simultaneously in real time. Because of the real-time distribution of substrates, intermediates and products between alternative metabolic pathways is not accessible by the usual analytical means, the present invention incorporates an MFA method with “on-line” or “real-time” metabolome data. Therefore, by calculation, the metabolic flux distributions during the fermentation can be quantified. The flux quantification and gene expression analysis, along with sophisticated experimental techniques, can be combined to upgrade the content of information in the physiological and genomic/proteomic data towards the unraveling of cellular function and regulation. This allows insight into metabolic pathways, which is highly desirable and necessary in order to understand the behavior of the organism.

Metabolic Flux Analysis (MFA) is an analysis technique for metabolic engineering. It has been used in connection with studies of cell metabolism where the aim is to direct as much carbon as possible from the substrate into the biomass and products. Example 1, below, generally describes an exemplary Metabolic Flux Analysis (MFA) that can be used in the methods of the invention.

“Metabolomics” is a relatively unexplored field and can encompass the analysis of all cellular metabolites. Metabolomics provides a powerful new tool for gaining insight into functional biology, and has provided snapshots of the levels of numerous small molecules within a cell, and how those levels change under different conditions. These studies are very complementary to gene and polypeptide expression studies (genomics and proteomics), which are actively being applied to studies of infectious diseases, production, and model organisms, as well as human cells and plants. The present invention provides an improved methodology to study “metabolomics” by providing a method for whole cell engineering of new or modified phenotypes by using real-time metabolic flux analysis.

In practicing the methods of the invention, cellular control can be studied at different hierarchical levels, at the level of the genome, at the level of the transcriptome, at the level of the proteome or at the level of the metabolome. Whilst there is much current interest in the genome-wide analysis of cells at the level of transcription (to define the ‘transcriptome’) and translation (to define the ‘proteome’), the third level of analysis, that of the ‘metabolome’, has been curiously unexplored to date. The term ‘metabolome’ refers to the entire complement of all the small molecular weight metabolites inside a cell suspension (or other sample) of interest. It is likely that measurement of the metabolome in different physiological states, particularly using the methods of the invention, will in fact be much more discriminating for the purposes of functional genomics.

The genome (the total genetic material in the cell) specifies an organism's total repertoire of responses. The genomes of several organisms have now been completely sequenced and several others are near completion or well under way (including a number of parasites). Of the genes so far sequenced via the systematic genome sequencing programs, the functions of fewer than half are known with any confidence. Technological advances now allow gene expression at any particular stage of development or in any particular physiological state to be analyzed. Such analyses can be carried out at the level of transcription using either Northern blots or, more efficiently, using hybridization array technologies to determine which genes are being expressed under different sets of conditions, i.e., the “transcriptome.” Similar analyses can be carried out at the level of translation to define the “proteome,” i.e., the total protein complement of the cell. Improvements in 2D electrophoresis and computer software for advanced image analysis allow 1-2×10³ proteins to be resolved on a single 20×20 cm plate; and, mass spectrometry coupled with database searching provides a method for rapid protein identification. Changes in the transcriptome represent the initial response of a cell to change, while changes in the proteome represent the final response at the level of the macromolecule. The third level of analysis, and one analyzed by the methods of the invention, is that of the “metabolome,” which includes the quantitative complement of all the low molecular weight molecules present in cells in a particular physiological or developmental state.

Metabolite levels, which are monitored in alternative aspects of the invention, are thus the variables of choice to measure in a quantitative analysis of cellular function. Metabolites,represent the down stream amplification of changes occurring in the transcriptome or the proteome. Moreover, metabolites regulate gene expression through a network of feedback pathways such that metabolites drive expression and act as the link between the genome and metabolism. The number of metabolites in the metabolome is also lower, by about an order of magnitude than the number of gene products in the transcriptome or the proteome (a typical eukaryotic cell contains around 10⁵ genes and 10⁴ different expressed proteins but only about 10³ different known metabolites). Therefore, in order to understand intermediary metabolism and to exploit this knowledge changes in the metabolome are much more relevant and will be much easier both to detect and to exploit than changes either in the transcriptome or the proteome.

The methods of the invention, by identifying sites of specific metabolic lesions via the metabolome, in addition to its inherent scientific interest, will lead to the detection of targets for potentially novel pharmaceuticals or agrochemicals in whole cells. The methods of the invention can also be used to design functional assays. From these results, they can enable the design of very much simpler assays in which only the targeted metabolites are studied for specific high throughput, mechanistic assays.

The metabolome analysis of the invention has the advantage of being an online non-invasive technology. While static metabolome analysis has some advantages over transcriptome and proteome analysis because, for many organisms, the number of metabolites was far fewer than the number of genes or proteins. However, static metabolome analysis had an intrinsic disadvantage as well. This was that while biochemistry could generate information about the metabolic pathways, there is no direct link between the metabolites and the genes. They were also problems in analyzing the concentration or even the very presence of certain metabolites. Current identification technologies such as infra-red spectrometry, mass spectrometry, or nuclear magnetic resonance spectroscopy produced some information but their use was limited and could not properly analyze a living cell. The methods of the invention, by providing “online” or “real-time” non-invasive technology solved this problem. The “online” or “real-time” time dimension of the methods of the invention, lacking in older techniques is one important factor in the methods ability to analyze a living cell.

Metabolic flux analysis (MFA) is a powerful analysis tool that can couple observed extracellular phenomena, such as uptake/excretion rates, growth rate, product and biomass yields, etc., with the intracellular carbon flux and energy distribution. The “on-line” or “real-time” MFA of the invention can be used to investigate the physiology of Escherichia coli, Saccharomyces cerevisiae, and hybridomas (see, e.g., Keasling (1998) Biotechnol. Bioeng. 5;58(2-3):231-239; Pramanik (1998) Biotechnol. Bioeng. 60(2):230-238; Nissen et al., 1997; Schulze et al., 1996; Follstad et al., 1999), lysine production and the effect of mutations in Corynebacterium glutamicum (see, e.g., Vallino (2000) Biotechnol. Bioeng. 67(6):872-885; Vallino and Stephanopoulos, 1993, 1994; Park et al., 1997; Dominguez (1998) Eur. J. Biochem. 254(l):96-102), riboflavin production in Bacillus subtilis (see, e.g., Sauer et al., 1996, 1998;- Sauer (1997) Nat. Biotechnol. 15:448-452), penicillin production in Penicillium chrysogenum (Nielsen (1995) Biotechnol. Prog. 11(3):299-305; Jorgensen (1995) Appl. Microbiol. Biotechnol. 43(l):123-130); and, peptide amino acid metabolism in Chinese hamster ovary (CHO) cells (see, e.g., Nyberg (1999) Biotechnol. Bioeng. 62(3):324-335; Nyberg (1999) Biotechnol. Bioeng. 62(3):336-347).

Moreover, the “on-line” or “real-time” MFA of the invention can be used in combination with NMR, MS, and/or GC-MS to yield hard to get information about futile cycles, the degree of reaction reversibility, as well as active pathways; see, e.g., Szyperski (1999) Metab. Eng. 1:189-197; Szyperski (1998) Q Rev. Biophys. 31:41-106; Szyperski (1995) Eur. J. Biochem. 232(2):433-448; Szyperski et al., 1997; Schmidt et al., 1998; Klapa (1999) Biotechnol. Bioeng. 62(4):375-391; Mollney et al., 1999; Park et al., 1999; Wiechert et al., 1999; Wittmann and Heinzle, 1999. Schilling, Edwards, and Palsson have even extended the use of MFA to include the analysis of genomic data and the structural properties of cellular networks (Schilling (2000-2001) Biotechnol. Bioeng. 71(4):286-306; Edwards and Palsson, 1998; Schilling et al., 1999a,b); to monitor the C(3)-C(4) metabolite interconversion at the anaplerotic node in many microorganisms (see, e.g., Petersen (2000) J. Biol. Chem. 275(46):35932-35941).

In MFA, the intracellular fluxes are calculated using a stoichiometric model for all the major intracellular reactions and by applying mass balances around the intracellular metabolites. As input to the calculations, a set of measured fluxes, typically the uptake rates of substrates and secretion rates of metabolites is used The novel “real-time” or “on-line” metabolic flux analysis of the invention can provide data regarding a full suite of metabolites synthesized by a biological system under given environmental conditions and/or with genetic regulation. The “real-time” or “on-line” MFA methods of the invention can provide metabolomic data sets that are extremely complex. The MFA methods of the invention can be an adequate tool to handle, store, normalize, and evaluate the acquired data in order to describe the systemic response of a complex biological system. FIG. 2 is a schematic illustrating the invention's new application of MFA to determine new phenotypes, pathway utilizations and cell responses to the studied strains during actual cell culture or fermentation periods. The results can be either used for post-fermentation analysis, or immediate control of the metabolism. The “on-line,” or “real-time” methods of the invention can also incorporate other analytical devices, such as HPLC and GC/MS, to estimate flux distribution in metabolic networks (constructed with our biochemical knowledge and genomic/proteomic information database) from experimental measurements. With these devices, “snapshots” of the biological systems under study can be obtained periodically, e.g., about every 1, 5, 10, 15, 20, 25, or 30 minutes, depending on the number of metabolic parameters studied and number of devices used.

Vector r for Metabolome Data

The on-line MFA of the invention uses “rate of change” data, or the difference between current metabolic measurements and last measurements. The differences are calculated and stored in the “raw measurement” vector for error analysis before they can be used. Thus, in one aspect, a “preprocessing unit” is used to filter out the errors for the measurement before the metabolic flux analysis to make sure that quality data be used. See Example 1, below.

Computer Systems

In one aspect, the methods of the invention use computer-implemented methods/programs to real time monitor the change in measured metabolic parameters over time. The methods of the invention can be practiced using any program language or computer/processor and in conjunction with any known software or methodology. For example, one of the programs called MATHEMATICA™ (Wolfram Research, Inc., Champaign, Ill.), such as MATHEMATICA 4.1™, or variations thereof, can be used, see Example 1, below; and, see also, e.g., Jamshidi (2001) Bioinformatics 17(3):286-287; Wilson (2001) Biophys. Chem. 91(3):281-304; Torrecilla (2001) J. Neurochem. 76(5):1291-1307.

The computer/processor used to practice the methods of the invention can be a conventional general-purpose digital computer, e.g., a personal workstation or portable computer, including various computer devices such as microprocessor, machine-readable memory units, and data transfer buses, a graphic controller, and one or more display devices such as CRT or LCD monitors. In addition, the computer may include data acquisition interface with sensing subsystem for receiving real-time measurements data and control interface which sends out computer-generated control commands to the controllable cell environment or the cell modification subsystem, either directly or indirectly via some other control units. Examples of the memory units include any form of memory elements, such as dynamic random access memory, flash memory or the like, or mass storage devices such as a magnetic disk drive, and optical disk drive. Computer software may be, at least in part, stored in one or more suitable memory units.

For example, a conventional personal computer such as those based on an Intel microprocessor and running a Windows operating system can be used. Any hardware or software configuration can be used to practice the methods of the invention. For example, computers based on other well-known microprocessors and running operating system software such as UNIX, Linux, MacOS and others are contemplated.

Improved Methods for Cellular Engineering, Protein Expression Profiling, Differential Labeling of Peptides, and Novel Reagents Therefor

The invention provides methods for simultaneously identifying individual proteins in complex mixtures of biological molecules and quantifying the expression levels of those proteins, e.g., proteome analyses. The methods compare two or more samples of proteins, one of which can be considered as the standard sample and all others can be considered as samples under investigation. The proteins in the standard and investigated samples are subjected separately to a series of chemical modifications, i.e., differential chemical labeling, and fragmentation, e.g., by proteolytic digestion and/or other enzymatic reactions or physical fragmenting methodologies. The chemical modifications can be done before, or after, or before and after fragmentation/digestion of the polypeptide into peptides.

Peptides derived from the standard and the investigated samples are labeled with chemical residues of different mass, but of similar properties, such that peptides with the same sequence from both samples are eluted together in the separation procedure and their ionization and detection properties regarding the mass spectrometry are very similar. Differential chemical labeling can be performed on reactive functional groups on some or all of the carboxy- and/or amino- termini of proteins and peptides and/or on selected amino acid side chains. A combination of chemical labeling, proteolytic digestion and other enzymatic reaction steps, physical fragmentation and/or fractionation can provide access to a variety of residues to general different specifically labeled peptides to enhance the overall selectivity of the procedure.

The standard and the investigated samples are combined, subjected to multidimensional chromatographic separation, and analyzed by mass spectrometry methods. Mass spectrometry data is processed by special software, which allows for identification and quantification of peptides and proteins.

Depending on the complexity and composition of the protein samples, it may be desirable, or be necessary, to perform protein fractionation using such methods as size exclusion, ion exchange, reverse phase, or other methods of affinity purifications prior to one or more chemical modification steps, proteolytic digestion or other enzymatic reaction steps, or physical fragmentation steps.

The combined mixtures of peptides are first separated by a chromatography method, such as a multidimensional liquid chromatography, system, before being fed into a coupled mass spectrometry device, such as a tandem mass spectrometry device. The combination of multidimensional liquid chromatography and tandem mass spectrometry can be called “LC-LC-MS/MS.” LC-LC-MS/MS was first developed by Link A. and Yates J. R., as described, e.g., by Link (1999) Nature Biotechnology 17:676-682; Link (1999) Electrophoresis 18:1314-1334.

In practicing the methods of the invention, proteins can be first substantially or partially isolated from the biological samples of interest. The polypeptides can be treated before selective differential labeling; for example, they can be denatured, reduced, preparations can be desalted, and the like. Conversion of samples of proteins into mixtures of differentially labeled peptides can include preliminary chemical and/or enzymatic modification of side groups and/or termini; proteolytic digestion or fragmentation; post-digestion or post-fragmentation chemical and/or enzymatic modification of side groups and/or termini.

The differentially modified polypeptides and peptides are then combined into one or more peptide mixtures. Solvent or other reagents can be removed, neutralized or diluted, if desired or necessary. The buffer can be modified, or, the peptides can be redissolved in one or more different buffers, such as a “MudPIT” (see below) loading buffer. The peptide mixture is then loaded onto chromatography column, such as a liquid chromatography column, a 2D capillary column or a multidimensional chromatography column, to generate an eluate.

The eluate is fed into a mass spectrograph, such as a tandem mass spectrograph. In one aspect, an LC ESI MS and MS/MS analysis is complete. Finally, data output is processed by appropriate software using database searching and data analysis.

In practicing the methods of the invention, high yields of peptides can generated for mass spectrograph analysis. Two or more samples can be differentially labeled by selective labeling of each sample. Peptide modifications, i.e., labeling, are stable. Reagents having differing masses or reactive groups can be chosen to maximize the number of reactive groups and differentially labeled samples, thus allowing for a multiplex analysis of sample, polypeptides and peptides. In one aspect, a “MudPIT” protocol is used for peptide analysis, as described herein. The methods of the invention can be fully automated and can essentially analyze every protein in a sample.

Unless defined otherwise, all technical and scientific terms used herein have the meaning commonly understood by a person skilled in the art to which this invention belongs. As used herein, the following terms have the meanings ascribed to them unless specified otherwise.

As used herein, the term “alkyl” is used to refer to a genus of compounds including branched or unbranched, saturated or unsaturated, monovalent hydrocarbon radicals, including substituted derivatives and equivalents thereof In one aspect, the hydrocarbons have from about 1 to about 100 carbons, about 1 to about 50 carbons or about 1 to about 30 carbons, about 1 to about 20 carbons, about 1 to about 10 carbons. When the alkyl group has from about 1 to 6 carbon atoms, it is referred to as a “lower alkyl.” Suitable alkyl radicals include, e.g., structures containing one or more methylene, methine and/or methyne groups arranged in acyclic and/or cyclic forms. Branched structures have a branching motif similar to isopropyl, tert-butyl isobutyl, 2-ethylpropyl, etc. As used herein, the term encompasses “substituted alkyls.” “Substituted alkyl” refers to alkyl as just described including one or more functional groups such as lower alkyl, aryl, acyl, halogen (i.e., alkylhalos, e.g., CF3), hydroxy, amino, alkoxy, alkylamino, acylamino, thioamido, acyloxy, aryloxy, arylamino, aryloxyalkyl, mercapto, thia, aza, oxo, both saturated and unsaturated cyclic hydrocarbons, heterocycles and the like. These groups may be attached to any carbon of the alkyl moiety. Additionally, these groups may be peident from, or integral to, the alkyl chain.

The term “alkoxy” is used herein to refer to the to a COR group, where R is a lower alkyl, substituted lower alkyl, aryl, substituted aryl, arylalkyl or substituted arylalkyl wherein the alkyl, aryl, substituted aryl, arylalkyl and substituted arylalkyl groups are as described herein. Suitable alkoxy radicals include, for example, methoxy, ethoxy, phenoxy, substituted phenoxy, benzyloxy phenethyloxy, tert.-butoxy, etc.

The term “aryl” is used herein to refer to an aromatic substituent that may be a single aromatic ring or multiple aromatic rings which are fused together, linked covalently, or linked to a common group such as a methylene or ethylene moiety. The common linking group may also be a carbonyl as in benzophenone. The aromatic ring(s) may include phenyl, naphthyl, biphenyl, diphenylmethyl and benzophenone among others. The term “aryl” encompasses “arylalkyl.” “Substituted aryl” refers to aryl as just described including one or more functional groups such as lower alkyl, acyl, halogen, alkylhalos (e.g., CF3), hydroxy, amino, alkoxy, alkylamino, acylamino, acyloxy, phenoxy, mercapto and both saturated and unsaturated cyclic hydrocarbons which are fused to the aromatic ring(s), linked covalently or linked to a common group such as a methylene or ethylene moiety. The linking group may also be a carbonyl such as in cyclohexyl phenyl ketone. The term “substituted aryl” encompasses “substituted arylalkyl.”

The term “arylalkyl” is used herein to refer to a subset of “aryl” in which the aryl group is further attached to an alkyl group, as defined herein.

The term “biotin” as used herein refers to any natural or synthetic biotin or variant thereof, which are well known in the art; ligands for biotin, and ways to modify the affinity of biotin for a ligand, are also well known in the art; see, e.g., U.S. Pat. Nos. 6,242,610; 6,150,123; 6,096,508; 6,083,712; 6,022,688; 5,998,155; 5,487,975.

The phrase “labeling reagents which . . . do not differ in ionization and detection properties in mass spectrographic analysis” means that the amount and/or mass sequence of the labeling reagents can be detected using the same mass spectrographic conditions and detection devices.

The term “polypeptide” includes natural and synthetic polypeptides, or mimetics, which can be either entirely composed of synthetic, non-natural analogues of amino acids, or, they can be chimeric molecules of partly natural peptide amino acids and partly non-natural analogs of amino acids. The term “polypeptide” as used herein includes proteins and peptides of all sizes.

The term “sample” as used herein includes any polypeptide-containing sample, including samples from natural sources, or, entirely synthetic samples.

The term “column” as used herein means any substrate surface, including beads, filaments, arrays, tubes and the like.

The phrase “do not differ in chromatographic retention properties” as used herein means that two compositions have substantially, but not necessary exactly, the same retention properties in a chromatograph, such as a liquid chromatograph. For example, two compositions do not differ in chromatographic retention properties if they elute together, i.e., they elute in what a skilled artisan would consider the same elution fraction.

Differential Labeling of Peptides and Polypeptides

In practicing the methods of the invention, proteins and peptides are subjected to a series of chemical modifications, i.e., differential chemical labeling. The chemical modifications can be done before, or after, or before and after fragmentation/digestion of the polypeptide into peptides. Differential labeling reagents can differ in their isotope composition (i.e., isotopical reagents), in their structural composition (i.e., homologous reagents), but by a rather small fragment which change does not alter the properties stated above, i.e., the labeling reagent differ in molecular mass but do not differ in chromatographic retention properties and do not differ in ionization and detection properties in mass spectrographic analysis, and the differences in molecular mass are distinguishable by mass spectrographic analysis.

In one aspect of the invention, mixtures of polypeptides and/or peptides coming from the “standard” protein sample and the “investigated” protein sample(s) are labeled separately with differential reagents, or, one sample is labeled and other sample remains unlabeled. As noted above, these differential reagents differ in molecular mass, but do not differ in retention properties regarding the separation method used (e.g., chromatography) and the mass spectrometry methods used will not detect different ionization and detection properties. Thus, these differential reagents differ either in their isotope composition (i.e., they are isotopical reagents) or they differ structurally by a rather small fragment which change does not alter the properties stated above (i.e., they are homologous reagents).

Differential chemical labeling can include esterification of C-termini, amidation of C-termini and/or acylation of N-termini. Esterification targets C-termini of peptides and carboxylic acid groups in amino acid side chains. Amidation targets C-termini of peptides and carboxylic acid groups in amino acid side chains. Amidation may require protection of amine groups first. Acylation targets N-termini of peptides and amino and hydroxy groups in amino acid side chains. Acylation may require protection of carboxylic groups first.

The skilled artisan will recognize that the chemical syntheses and differential chemical labeling of peptides and polypeptides (e.g., esterification, amidation, and acylation) used to practice the methods of the invention can be by a variety of procedures and methodologies, which are well described in the scientific and patent literature, e.g., Organic Syntheses Collective Volumes, Gilman et al. (Eds), John Wiley & Sons, Inc., NY; Venuti (1989) Pharm. Res. 6: 867-873; the Beilstein Handbook of Organic Chemistry (Beilstein Institut fuer Literatur der Organischen Chemie, Frankfurt, Germany); Beilstein online database and references obtainable therein; “Organic Chemistry,” Morrison & Boyd, 7th edition, 1999, Prentice-Hall, Upper Saddle River, N.J. The invention can be practiced in conjunction with any method or protocol known in the art, which are well described in the scientific and patent literature. For example, the esterification, amidation, and acylation reactions may be performed on the mixtures of peptides in a fashion similar to other reaction of these types already described in prior art, such as:

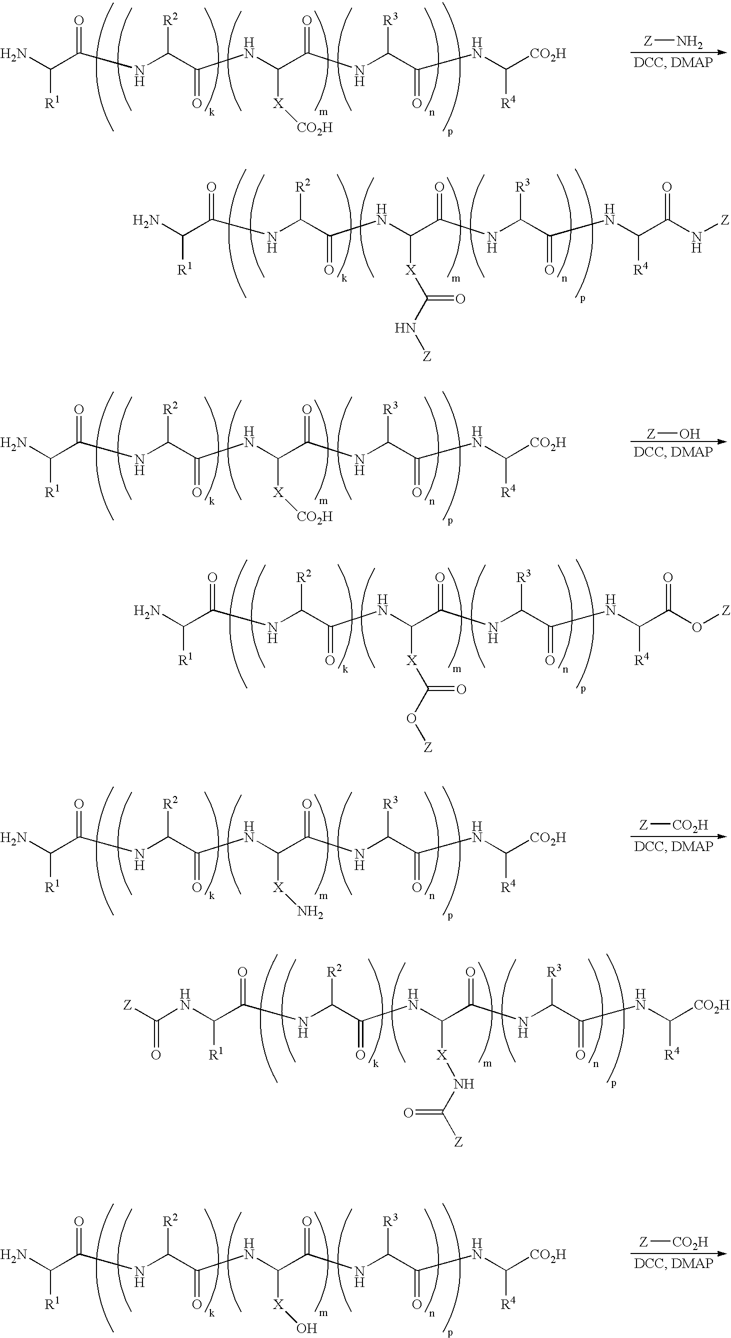

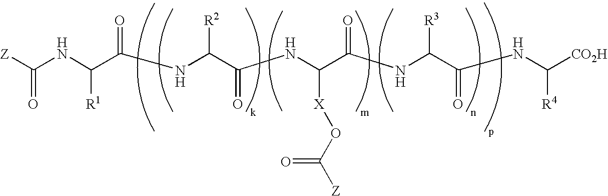

In alternative aspects, reagents comprise the general formulae:

i. Z^{A}OH and Z^{B}OH to esterify peptide C-terminals and/or Glu and Asp side chains;

ii. Z^{A}NH₂/Z^{B}NH₂ to form amide bond with peptide C-terminals and/or Glu and Asp side chains; or

iii. Z^{A}CO₂H/Z^{B}CO₂H to form amide bond with peptide N-terminals and/or Lys and Arg side chains;

wherein Z^{A} and Z^{B} independently of one another can be R-Z¹-A¹-Z²-A²-Z³-A³-Z⁴-A⁴- , and Z¹, Z², Z³, and Z⁴ independently of one another can be selected from O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, OB(OR)(OR¹), or, Z¹, Z², Z³, and Z⁴ independently of one another may be absent, and R is an alkyl group; and,

A¹, A², A³, and A⁴ independently of one another can be selected from (CRR¹)n, and R is an alkyl group. In alternative aspects, some single C—C bonds from (CRR¹)n may be replaced with double or triple bonds, in which case some groups R and R¹ will be absent, (CRR¹)n can be an o-arylene, an m-arylene, or a p-arylene with up to 6 substituents, carbocyclic, bicyclic, or tricyclic fragments with up to 8 atoms in the cycle with or without heteroatoms (O, N, S) and with or without substituents, or A¹, A², A³, and A⁴ independently of one another can be absent; R, R¹, independently from other R and R¹ in Z¹-Z⁴ and independently from other R and R¹ in A¹-A⁴, can be hydrogen, halogen or an alkyl group, such as an alkenyl, an alkynyl or an aryl group;

n in Z¹-Z⁴, independent of n in A¹-A⁴, is an integer that can have value from 0 to about 51; 0 to about 41; 0 to about 31; 0 to about 21, 0 to about 11; 0 to about 6;

In alternative aspects, Z^{A} has the same structure as Z^{B}, but they have different isotope compositions. Any isotope may be used. In alternative aspects, if Z^{A} contains x number of protons, Z^{B} may contain y number of deuterons in the place of protons, and, correspondingly, x-y number of protons remaining; and/or if Z^{A} contains x number of borons-10, Z^{B} may contain)y number of borons-11 in the place of borons-10, and, correspondingly, x-y number of borons-10 remaining; and/or if Z^{A} contains x number of carbons-12, Z^{B} may contain y number of carbons-13 in the place of carbons-12, and, correspondingly, x-y number of carbons-12 remaining; and/or if Z^{A} contains x number of nitrogens-14, Z^{B} may contain y number of nitrogens-15 in the place of nitrogens-14, and, correspondingly, x-y number of nitrogens-14 remaining; and/or if Z^{A} contains x number of sulfurs-32, Z^{B} may contain y number of sulfurs-34 in the place of sulfurs-32, and, correspondingly, x-y number of sulfurs-32 remaining; and so on for all elements which may be present and have different stable isotopes; x and y are whole numbers such that x is greater than y. In one aspect, x and y are between 1 and about 11, between 1 and about 21, between 1 and about 31, between I and about 41, between 1 and about 51.

In alternative aspects, reagent pairs/series comprise the general formulae:

i. CD₃(CD₂)ₙOH/CH₃(CH₂)ₙOH to esterify peptide C-terminals, where n=0, 1, 2, . . . , y; (delta mass=3+2n);

ii. CD₃(CD₂)ₙNH₂/CH₃(CH₂)ₙNH₂ to form amide bond with peptide C-terminals where n=0, 1, 2, . . . , y (delta mass=3+2n);

iii. D(CD₂)ₙCO₂H/H(CH₂)ₙCO₂H to form amide bond with peptide N-terminals, where n=0, 1, 2, . . . , y (delta mass=1+2n);

wherein y is an integer that can have value of about 51; about 41; about 31; about 21, about 11; about 6, or between about 5 and 51.

Other exemplary reagents can be presented by general formulae:

i. Z^{A}OH and Z^{B}OH to esterify peptide C-terminals;

ii. Z^{A}NH₂/Z^{B}NH₂ to form an amide bond with peptide C-terminals;

iii. Z CO₂H/Z^{B}CO₂H to form an amide bond with peptide N-terminals;

wherein Z^{A} and Z^{B} can be R-Z¹-A¹-Z²-A²-Z³-A³-Z⁴-A⁴- and Z¹, Z², Z³, and Z⁴, independently of one another, can be selected from O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, or OB(OR)(OR¹); or, Z¹, Z², Z³, and Z⁴, independently of one another, can be absent, and, R is an alkyl group;

A¹, A², A³, and A⁴, independently of one another, can be a moiety comprising the general formulae (CRR¹)n. In alternative aspects, single C—C bonds in some (CRR¹)n groups may be replaced with double or triple bonds, in which case some groups R and R¹ will be absent, or (CRR¹)n can be an o-arylene, an m-arylene, or a p-arylene with up to 6 substituents, or a carbocyclic, a bicyclic, or a tricyclic fragments with up to 8 atoms in the cycle, with or without heteroatoms (e.g., O, N or S atoms), or, with or without substituents, or, A¹-A⁴ independently of one another may be absent;

In alternative aspects, R, R¹, independently from other R and R¹ in Z¹-Z⁴ and independently from other R and R¹ in A¹-A⁴, can be a hydrogen atom, a halogen or an alkyl group, such as an alkenyl, an alkynyl or an aryl group;

In alternative aspects, n in Z¹ -Z⁴ is independent of n in A¹-A⁴ and is an integer that can have value of about 51; about 41; about 31; about 21, about 11; about 6.

In alternative aspects, Z has a similar structure to that of Z^{B}, but Z^{A} has x extra —CH₂— fragment(s) in one or more A¹-A⁴ fragments, and/or Z^{A} has x extra —CF₂— fragment(s) in one or more A¹-A⁴ fragments. Alternatively, Z^{A} can contain x number of protons and Z^{B} may contain y number of halogens in the place of protons. Alternatively, where Z^{A} contains x number of protons and Z^{B} contains y number of halogens, there are x-y number of protons remaining in one or more A¹-A⁴ fragments; and/or Z^{A} has x extra —O— fragment(s) in one or more A¹-A⁴ fragments; and/or Z^{A} has x extra —S— fragment(s) in one or more A¹-A⁴ fragments; and/or if Z^{A} contains x number of —O— fragment(s), Z may contain y number of —S— fragment(s) in the place of —O— fragment(s), and, correspondingly, x-y number of —O— fragment(s) remaining in one or more A¹-A⁴ fragments; and the like.

In alternative aspects, x and y are integers that can have value of between 1 about 51; of between 1 about 41; of between 1 about 31; of between 1 about 21, of between 1 about 11; of between 1 about 6, such that x is greater than y.

Exemplary homologous reagents pairs/series are

i. CH₃(CH₂)ₙOH/CH₃(CH₂)ₙ₊ₘOH to esterify peptide C-terminals, where n=0, 1, 2, . . . , y; m=1, 2, . . . , y (delta mass=14m)

ii. CH₃(CH₂)ₙNH₂/CH₃(CH₂)ₙ₊ₘNH₂ to form amide bond with peptide C-terminals, where n=0, 1, 2, . . . , y; m=1, 2, . . . , y (delta mass=14m)

iii. H(CH₂)ₙCO₂H/H(CH₂)ₙ₊ₘCO₂H to form amide bond with peptide N-terminals, where n=0, 1, 2, . . . , y; m=1, 2, . . . , y (delta mass=14m)

wherein y is an integer that can have value of about 51; about 41; about 31; about 21, about 11; about 6, or between about 5 and 51.

Methods for Peptide/Protein Separation and Detection

The methods of the invention use chromatographic techniques to separate tagged polypeptides and peptides. In one aspect, a liquid chromatography is used, e.g., a multidimensional liquid chromatography. The chromatogram eluate is coupled to a mass spectrometer, such as a tandem mass spectrometry device (e.g., a “LC-LC-MS/MS” system). Any variation and equivalent thereof can be used to separate and detect peptides. LC-LC-MS/MS was first developed by Link A and Yates J. R., as described, e.g., in (Link (1999) Nature Biotechnology 17:676-682; Link (2000) Electrophoresis 18, 1314-1334. In one aspect, the LC-LC-MS/MS technique is used; it is effective for complexed peptide separation and it is easily automated. LC-LC-MS/MS is commonly known by the acronym “MudPIT,” for “Multi-dimensional Protein Identification Technique.”

Variations and equivalents of LC-LC-MS/MS used in the methods of the invention include methodologies involving reversed phase columns coupled to either cation exchange columns (as described, e.g., by Opiteck (1997) Anal. Chem. 69:1518-1524; or, size exclusion columns (as described, e.g., by Opiteck (1997) Anal. Biochem. 258:349-361). In one aspect, an LC-LC-MS/MS technique uses a mixed bed microcapillary column containing strong cation exchange (SCX) and reversed phase (RPC) resins. Other exemplary alternatives include protein fractionation combined with one-dimensional LC-ESI MS/MS or peptide fractionation combined MALDI MS/MS.

Depending on the complexity or the property of the protein samples, any protein fractionation method, including size exclusion chromatography, ion exchange chromatography, reverse phase chromatography, or any of the possible affinity purifications, can be introduced prior to labeling and proteolysis. In some circumstances, use of several different methods may be necessary to identify all proteins or specific proteins in a sample.

Sequence Analysis and Quantification

Both quantity and sequence identity of the protein from which the modified peptide originated can be determined by a mass spectrometry device, such as a “multistage mass spectrometry” (MS). This can be achieved by the operation of the mass spectrometer in a dual mode in which it alternates in successive scans between measuring the relative quantities of peptides eluting from the capillary column and recording the sequence information of selected peptides. Peptides are quantified by measuring in the MS mode the relative signal intensities for pairs or series of peptide ions of identical sequence that are tagged differentially, which therefore differ in mass by the mass differential encoded within the differential labeling reagents.

Peptide sequence information can be automatically generated by selecting peptide ions of a particular mass-to-charge (m/z) ratio for collision-induced dissociation (CID) in the mass spectrometer operating in the tandem MS mode, as described, e.g., by Link (1997) Electrophoresis 18:1314-1334;Gygi (1999) Nature Biotechnol. 17:994-999; Gygi (1999) Cell Biol. 19:1720-1730.

The resulting tandem mass spectra can be correlated to sequence databases to identify the protein from which the sequenced peptide originated. Exemplary commercial available softwares include TURBO SEQUEST™ by Thermo Finnigan, San Jose, Calif.; MASSSCOT™ by Matrix Science, SONAR MS/MS™ by Proteometrics. Routine software modifications may be necessary for automated relative quantification.

Mass Spectrometry Devices

In the methods of the invention use mass spectrometry to identify and quantify differentially labeled peptides and polypeptides. Any mass spectrometry system can be used. In one aspect of the invention, combined mixtures of peptides are separated by a chromatography method comprising multidimensional liquid chromatography coupled to tandem mass spectrometry, or, “LC-LC-MS/MS,” see, e.g., Link (1999) Biotechnology 17:676-682; Link (1999) Electrophoresis 18:1314-1334. Exemplary, mass spectrometry devices include those incorporating matrix-assisted laser desorption-ionization-time-of-flight (MALDI-TOF) mass spectrometry (see, e.g., Isola (2001) Anal. Chem. 73:2126-2131; Van de Water (2000) Methods Mol. Biol. 146:453-459; Griffin (2000) Trends Biotechnol. 18:77-84; Ross (2000) Biotechniques 29:620-626, 628-629). The inherent high molecular weight resolution of MALDI-TOF MS conveys high specificity and good signal-to-noise ratio for performing accurate quantitation.

Use of mass spectrometry, including MALDI-TOF MS, and its use in detecting nucleic acid hybridization and in nucleic acid sequencing, is well known in the art, see, e.g., U.S. Pat. Nos. 6,258,538; 6,238,871; 6,238,869; 6,235,478; 6,232,066; 6,228,654; 6,225,450; 6,051,378; 6,043,031.

Fragmentation and Proteolytic Digestion

In practicing the methods of the invention, polypeptides are fragmented, e.g., by proteolytic, i.e., enzymatic, digestion and/or other enzymatic reactions or physical fragmenting methodologies. The fragmentation can be done before and/or after reacting the peptides/polypeptides with the labeling reagents used in the methods of the invention.

Methods for proteolytic cleavage of polypeptides are well known in the art, e.g., enzymes include trypsin (see, e.g., U.S. Pat. Nos. 6,177,268; 4,973,554), chymotrypsin (see, e.g., U.S. Pat. Nos. 4,695,458; 5,252,463), elastase (see, e.g., U.S. Pat. No. 4,071,410); subtilisin (see, e.g., U.S. Pat. No. 5,837,516) and the like.

In one aspect, a chimeric labeling reagent of the invention includes a cleavable linker. Exemplary cleavable linker sequences include, e.g., Factor Xa or enterokinase (Invitrogen, San Diego Calif.). Other purification facilitating domains can be used, such as metal chelating peptides, e.g., polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.).

Biological Samples

The methods are based on comparison of two or more samples of proteins, one of which can be considered as the standard sample and all others can be considered as samples under investigation. For example, in one aspect, the invention provides a method for quantifying changes in protein expression between at least two cellular states, such as, an activated cell versus a resting cell, a normal cell versus a cancerous cell, a stem cell versus a differentiated cell, an injured cell or infected cell versus an uninjured cell or uninfected cell; or, for defining the expressed proteins associated with a given cellular state.

Sample can be derived from any biological source, including cells from, e.g., bacteria, insects, yeast, mammals and the like. Cells can be harvested from any body fluid or tissue source, or, they can be in vitro cell lines or cell cultures.

Detection Devices and Methods

The devices and methods of the invention can also incorporate in whole or in part designs of detection devices as described, e.g., in U.S. Pat. Nos. 6,197,503; 6,197,498; 6,150,147; 6,083,763; 6,066,448; 6,045,996; 6,025,601; 5,599,695; 5,981,956; 5,698,089; 5,578,832; 5,632,957.

Lipidomic Profiling of Microbes

The invention provides differential profiling of lipid specie as a process to “fingerprint” different microbial species. This methodology can be employed to assess the physiological state of a single bacterial culture or population. The process takes advantage of the fact that many different organisms have substantial differences in lipid composition of their plasma membranes. The process of the invention takes advantage of the combinatorial information contained within triglycerides, significantly advancing previously used methods, such as FAME (fatty acid methyl ester analysis) to type bacteria. The process of the invention uses a combination of lipid specific extraction procedures, advanced high-resolution nanospray mass spectrometry with spectral matching algorithms. This invention provides a rapid means to type bacterial cultures, or as a rapid quality control of cultures. The advantage of this method over the standard 16S sequencing methods is speed. Using workflow automation, at least 100 samples can be processed in a hour on a single instrument.

The typing of bacterial cultures is commonly performed using the now obsolete FAME analysis and 16S typing. 16S typing is commonly performed using PCR to amplify a stretch of DNA, followed by nucleotide sequencing of the DNA. Alternative methods, such as hybridization of bacterial DNA against an array of select 16S targets also exist. Only sequencing can provide information to determine phylogenetic relationships, however, this method is time-consuming as a routine analysis method. See, e.g., U.S. Pat. No. 5,776,723, describing M. tuberculosis detection using fatty acid profiling, and Diagn Microbiol Infect Dis December 2000;38(4):213-221; Gut February 2000;48(2):198-205; Appl Environ Microbiol April 2000;66(4):1668-75; Int J Syst Bacteriol April 1996;46(2):466-9.

The method of the invention takes advantage of the combinatorial information stored within lipid molecules, and the fact that many different bacterial species have different lipid compositions. Furthermore, the synthesis and modification of lipids depend on the metabolic state of cells, thus providing additional information about the cellular state and metabolism.

Specifically, the method of the invention employs a lipid extraction procedure (see appendix), followed by determining the composition by mass spectrometry (see appendix). The data can be stored and “fingerprinted”. This fingerprinting will discard common information and save masses and abundances of characteristic and unique lipid species. Every new mass spectrum can thus be matched against a database of characteristic fingerprints for species typing.

Every lipid molecule is a result of a biochemical synthesis catalyzed by enzymes. Since the metabolic pathways of lipid synthesis and modification are well understood, one can map the species identified by mass spectrometer analysis to known pathways. The information derived from this cross-correlation can be exploited as a descriptor of the metabolic state of a cell. This is especially useful, because the lipid profile is subject to cellular stresses, nutrient availability and growth phase.

The method of the invention is superior over the classical FAME methods (fatty acid methyl ester analysis), because it preserves the combinatorial complexity of lipids. FAME reduces the complexity of the lipidome by creating chemical derivatives of fatty acids. Since a phospholipid or triglyceride consists of a head-group, and two or three fatty acid tails, and since headgroups and fatty acyl species can be different, the sum of all lipids is orders of magnitudes more complex than the sum of all fatty acids.

Unlike other mass spectrometry-based methods based on fast atom bombardment or MALDI of whole bacteria, the method of the invention is more sensitive and can analyze much more complex profiles.

This aspect of the invention can be practiced in conjunction with GC-MS (FAME ANALYSIS) or electrospray-MS. The later has been used to measure intact lipids, including lipids consisting of two to three fatty acid moieties. Since intact lipids capture the combinatorial space of fatty acids and head-groups, there is a greater diversity in lipid species than fatty acid species alone. The invention measures a “fingerprint” of intact lipids. This information can be correlated to species identity and/or the growth environment of the species. This method combines the concept of FAME with the more detailed measurement of intact lipid species.

An exemplary lipid extraction protocol for practicing the methods of the invention is described in Example 5, below.

Monitoring Changes in Protein Profiles and Activity in Whole Cell Engineering

The invention provides novel proteomics strategies for simplifying complex protein mixtures and to quantitatively analyze the simplified mix to identify proteins that are significantly different in amount. The invention further provides methods to modify cell populations. The invention establishes a connected liquid chromatography and mass spectrometer platform to measure differential protein levels and identify differentially expressed proteins by protein sequencing. Thus, one aspect of the invention comprises a system comprising connected liquid chromatography and mass spectrometer platform(s) to measure differential protein levels and identify differentially expressed proteins by protein sequencing.

In alternative aspects, the methods employ sub cellular fractionation by FPLC, differential ICAT labeling, and/or enzymatic digestion to generate peptides. In one aspect, this is followed by two-dimensional HPLC separation and/or ES-MS/MS. This strategy provides a comprehensive platform to identify quantitative differences in complex protein mixtures, and identify the peptide and corresponding proteins by mass spectral sequencing.

In one aspect the mass and sequence information is encoded onto a database. Thus, the methods provide a computer program product with a user interface comprising the mass and sequence information. The database of the invention can be submitted for database searches to public and private genome databases to identify a corresponding gene, if any.

In cases where the genomic sequence is not known the differentially expressed proteins are sequenced directly on the mass spectrometer. All acquired data can be collected and stored in a database structure for compilation and subsequent data mining.

These methods are employed with whole cell optimization methods. Thus, the invention provides a highly sophisticated and interconnected network of monitoring and design tools to create cells with novel genetic and physiological traits. The systems and methods of the invention can be used to custom design an organism to meet a certain beneficial requirement in a process or environment.

To obtain design targets from whole cell systems, representative features of a cell at the “omics” level, such as all expressed genes, all expressed proteins and metabolites in mutants or wild type strains, or strains grown under different conditions, are measured. These cellular building blocks are correlated to a particular phenotype. The invention combines these comparative measurements with a knowledgebase of existing information to extract essential information of how organisms adopt to an environment or task, and what the bottlenecks are. This information is used to make the necessary adjustments and changes to the genetic code of the organisms to improve the bottlenecks and to introduce desirable feats. The new organism are evaluated by monitoring the desired property in assays, e.g., by RNA expression profiling and proteome analysis. Finally, the new organisms are evaluated by testing the fitness of the organism under industrial conditions, e.g. fermentation.

Multidimensional Micro Liquid Chromatography MS/MS (μLC-MS/MS)

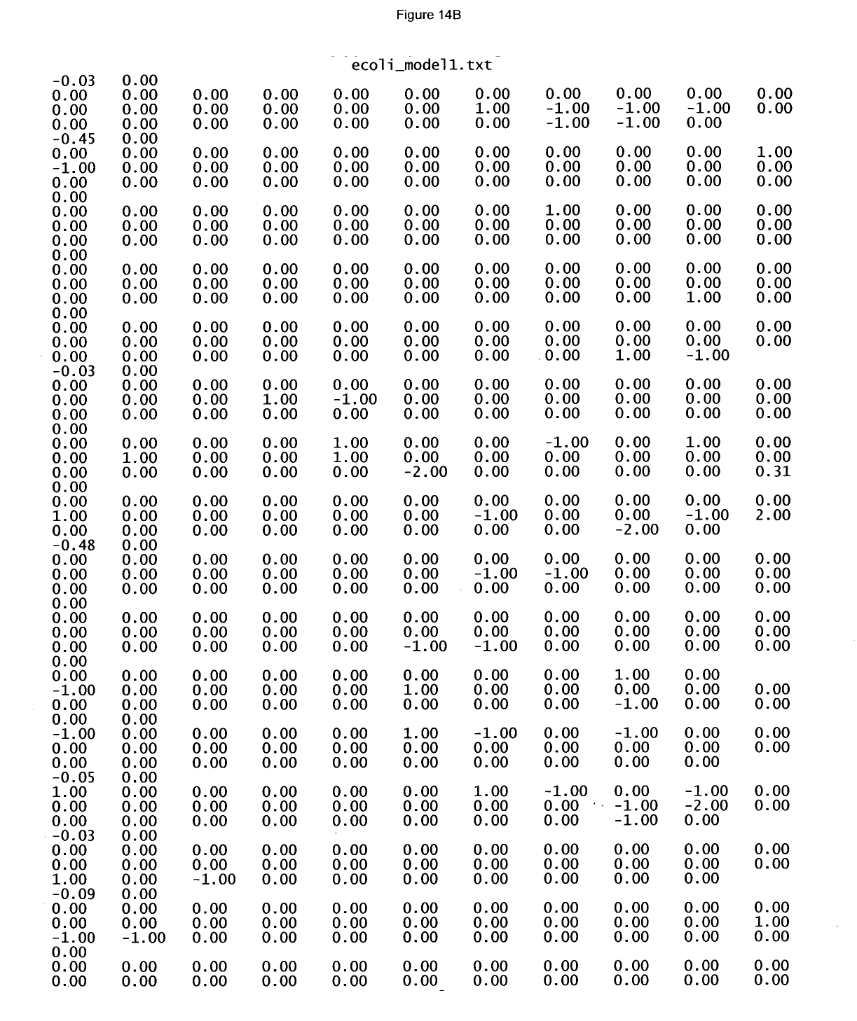

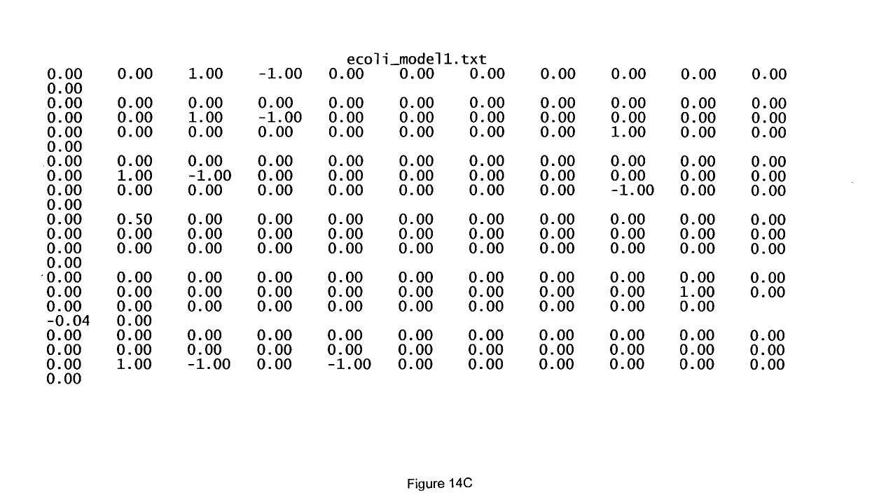

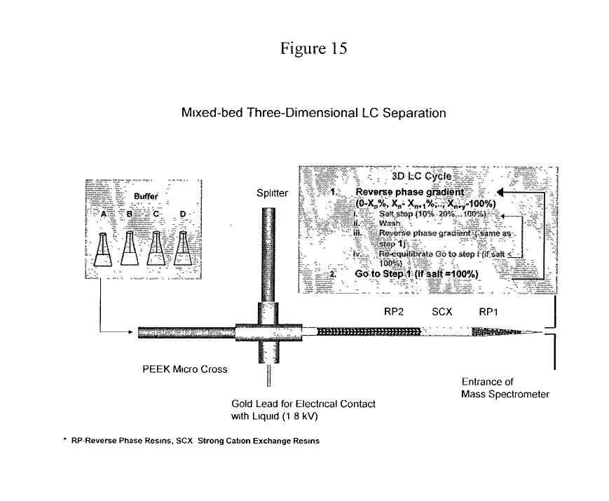

The invention further provides methods and systems comprising multidimensional micro liquid chromatography MS/MS (μLC-MS/MS) configurations. Multidimensional micro liquid chromatography MS/MS systems of the invention can be coupled to a bioinformatics analysis environment. The μLC-MS/MS system can be used for proteomics in a high throughput and fully automated manner. This technique can be used to identify a wide array of proteins regardless of pI or molecular weight. Moreover, in contrast to conventional 2D gel methods, this approach can access hydrophobic proteins and low abundant proteins. In addition, the 3D μLC MS/MS technology of the invention can be highly sensitive, have substantial peak capacity, and, in one aspect, can provide a dynamic range greater than about 10,000 to 1. An exemplary multidimensional micro liquid chromatography MS/MS (μLC-MS/MS) configuration is illustrated in FIG. 15.

An exemplary feature of the 3D μLC MS/MS system of the invention is the in-house constructed three-dimensional (3-D) microcapillary columns that are used for liquid chromatography. FIG. 15 shows a diagram of an exemplary microcapillary column and depicts the configuration of resins that are packed into the column to achieve 3-D separations. The systems and methods of the invention provide good separations of complex peptide mixtures using a configuration of reverse phase (RP1), strong cation exchange (SCX), and reverse phase (RP2) resins.

FIG. 15 also shows that various gradient elution schemes can be used to achieve optimal peptide separations. Without desalting, the total peptide mixture can be directly loaded onto a 3-D microcapillary column. A discrete fraction of the absorbed peptides can be displaced from the RP2 to the SCX section using a reverse phase gradient (Xn−Xn+1%). This fraction of peptides can be retained onto the SCX section and then sub-fractionated from the SCX column onto the RPC column using a step gradient of salt, where part of the peptides are eluted and retained on the RP1 section while contaminating salts and buffers are washed through. The sub-fractionated peptides can be separated on the RP1 column using the same reverse phase gradient (Xn−Xn+1l/%). The masses and sequences of separated and eluted peptides can be directly detected by a tandem mass spectrometer. This process can be repeated using increasing salt concentration to displace additional sub-fractions from the SCX column following each step by a reverse phase gradient.

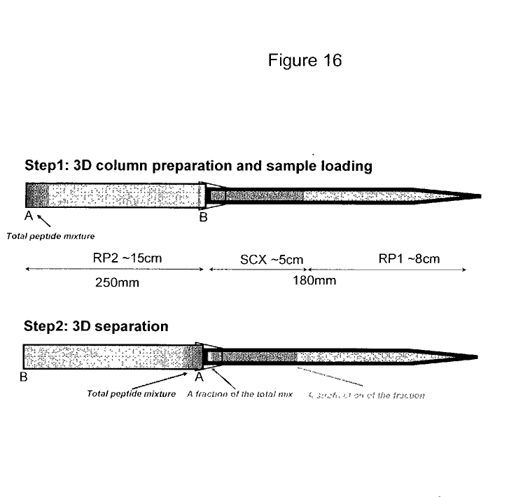

Upon the completion of the whole sequence of salt steps, the process can be repeated, employing a higher reverse phase gradient (e.g., Xn+1−Xn+2%, Xn+2>Xn+1, n=0, 1, 2, 3 . . . , X1=0). Each of the cycles can be applied in an iterative manner, with the total number of cycles depending on the complexity of the peptides. The processing of a complex protein mixture can involve about 3-6 acetonitrile cycles followed by 6-12 salt gradient steps. The MS/MS data from all of the fractions can be analyzed by database searching. FIGS. 15 and 16 illustrate this exemplary 3D LC set-up and process. FIG. 16 illustrates (as Step 1) an exemplary 3-D column preparation and sample loading and (as Step 2) a 3-D separation of an exemplary 3-D μLC MS/MS system of the invention.

Initial studies were carried out using exemplary 3D μLC MS/MS technology to profile a yeast proteome and a Streptomyces proteome. The goal of this project was to detect as many yeast or Streptomyces (S. diversa) proteins as possible in the complex peptide. Soluble, membrane-associated and integral membrane protein extracts were prepared from each sample. Extracts were treated sequentially with Lys-C and trypsin after reduction/alkylation with iodoacetamide in the presence of urea had been carried out. Peptide mixtures were then analyzed on the 3D μLC MS/MS system as described in detail in FIG. 2. This procedure has been proved to be effective for high peak capacity and high resolution separation. We used two separate columns to make a 3D column. The first RP and SCX were packed tandemly into an 180 μm capillary column and the second RP was packed into a 250 μm capillary column. These two columns were coupled together using a micro union. The total peptide mixture was loaded directly to the 3D column through RP2. The RP2 was then decoupled, flipped and the recoup led to SCX+RP1. The total peptide zone should be very close to the SCX region.

Protein identification was achieved by matching the MS/MS spectra acquired to the predicted protein sequences from either yeast or the Streptomyces (S. diversa). More than 1000 proteins can be identified from each 3D LC MS/MS experiment.

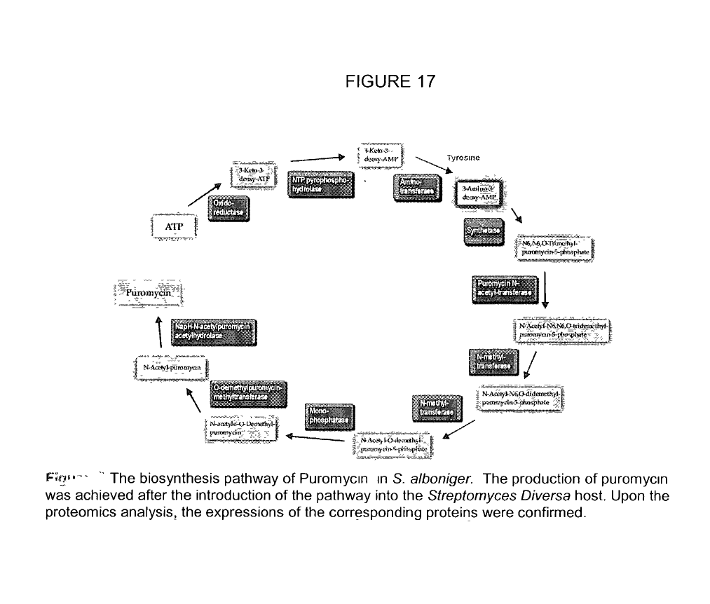

Heterologous expression of natural product biosynthetic pathways in Streptomyces (S. diversa) was also detected using the methods of the invention. FIG. 17 illustrates the biosynthetic pathway for the antibiotic puromycin. For these experiments, the DS10 strain of S. diversa was transformed with a plasmid containing all the genes required for puromycin synthesis to create the new strain DS10-puromycin. The goal of this study was to detect at least one peptide from all ten of the enzymes required for puromycin biosynthesis in the DS10-puromycin strain.

Soluble, membrane-associated and integral membrane protein extracts were prepared from strains DS10 and DS10-puromycin. Extracts were treated separately with Lys-C and trypsin after reduction/alkylation with iodoacetamide in the presence of urea had been carried out.

Table 1 shows the optimal esterification conditions for a model peptide:

TABLE 1
Optimal esterification conditions for model peptide

| Esterification | HCl concentration | Best Reaction Time Range |
|---|---|---|
| MeOH | 0.25 | 0.5 hour |
| EtOH | 0.5 | 1 hour |
| Iso-Propanol | 2 | 4 hour |

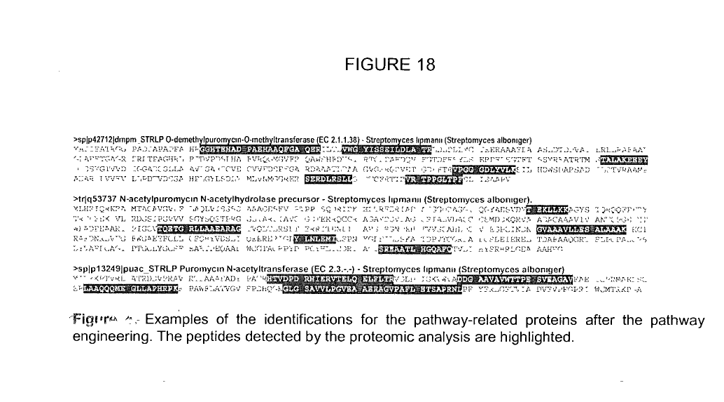

Peptide mixtures were then analyzed on the 3D μLC MS/MS system and protein identification was achieved by matching the MS/MS spectra acquired to the predicted protein sequences from both S. diversa and the components of the puromycin biosynthetic pathway. In extracts derived from soluble fractions of DS10-puromycin all ten unique proteins from the puromycin pathway were identified in this analysis. FIG. 18 illustrates representative peptides that were detected for three of the enzymes in the puromycin pathway. Note that multiple peptides were detected for each enzyme in the pathway leading to unambiguous identification of these proteins.

In addition, more than 800 soluble proteins were identified in both S. diversa strains. FIG. 18 illustrates examples of the identifications for the pathway-related proteins after pathway engineering. The peptides detected by proteomic analysis are highlighted.

Data Analysis Aspects of the Quantitative Proteomics Procedures:

In one aspect, the LC-MS or LC-LC-MS data acquired from the differentially labeled peptides is subjected to the following exemplary analyses, as set forth in 1 and 2 below. Analysis 1 is generally more accurate than analysis 2. However, both can be used in a quantitative proteomics analysis.

1. Component extraction, which is consisted of following sub-steps:

a. For every MS spectrum from the beginning of the LC elution, select the “significant” ions, which are above the local noise background and contain predominately C¹² isotopes.

b. For every “significant” ion, generate a “selected ion chromatogram” using the neighboring MS spectra. The width of the region should be at least 2× of the expected width of the peptide elution (D0).

c. Determine the peak location, quality, area and baseline level based on the “selected ion chromatogram”.

d. Save the “valid” component, which exceeds the quality requirement for the LC elution peak and locates within the elution boundary of the “significant” ion.

e. Link the components to the MS/MS spectra if available based on their m/z (mass to charge ratio) values and elution time with the consideration of appropriate tolerances.

2. Concurrently, if the MS/MS spectra of the peptides are acquired, the intensities of the precursor ions are extracted as follow:

a. The duplicated MS/MS spectra are identified using the following algorithm:

i. For every MS/MS spectrum from the beginning of the LC elution, compare it to all MS/MS spectra;

ii. The spectra equivalency is declared is the spectra pair satisfy the following requirements:

1. Their precursor m/z values are within the pre-defined tolerance;

2. Their elution times are within a pre-defined tolerance;

3. Their “signature” peaks achieved a pre-defined degree of match;

4. Their “dot-products” in both forward and backward direction exceed pre-defined thresholds.

b. The duplicated spectra are merged based on the m/z position of the peaks. The elution times of the first (T1) and last (T2) spectra are stored as a part of the description of the merged spectrum.

c. The intensity of the precursor ions is calculated from the MS1 spectra by integrating the region where the precursor ions are detected. This region is defined as (T1−D0/2, T1+D0/2), where D0 is defined as in 1.b.

3. Reconstruct the series of differentially labeled peptides based on the predictable elution behavior, in combination with the predicted mass differences.

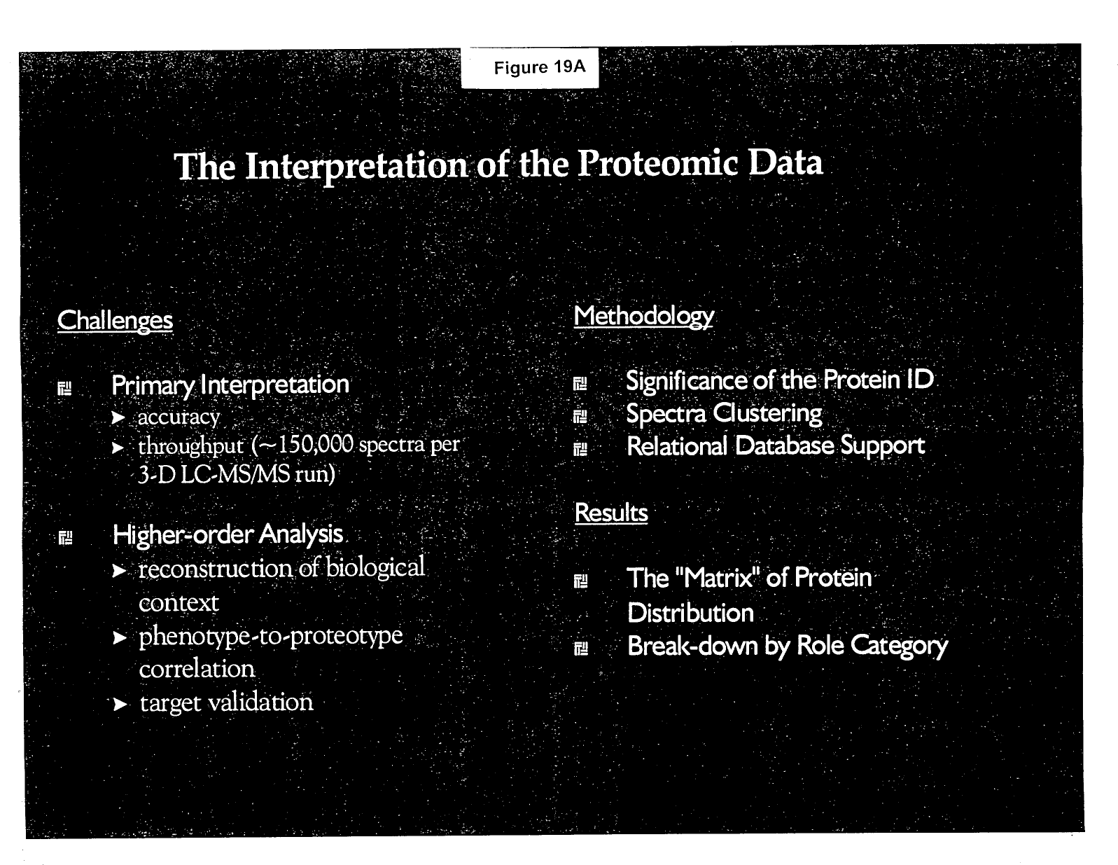

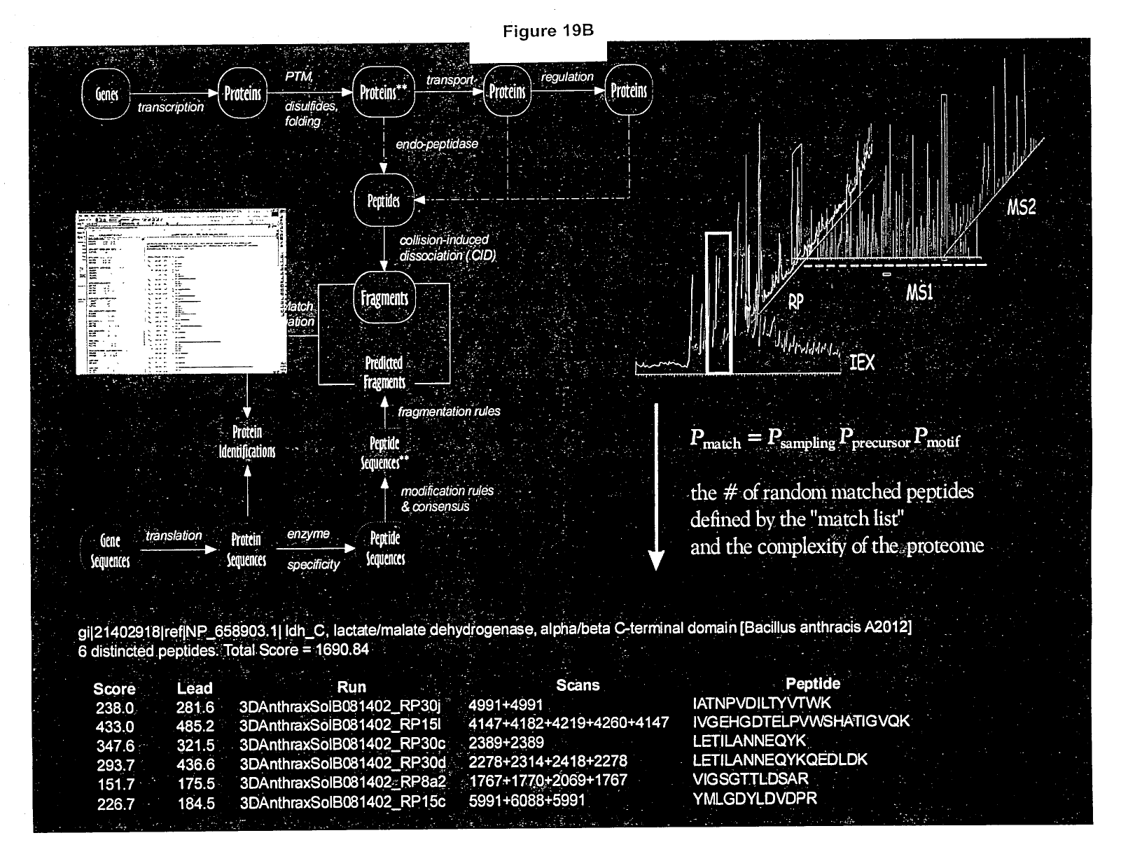

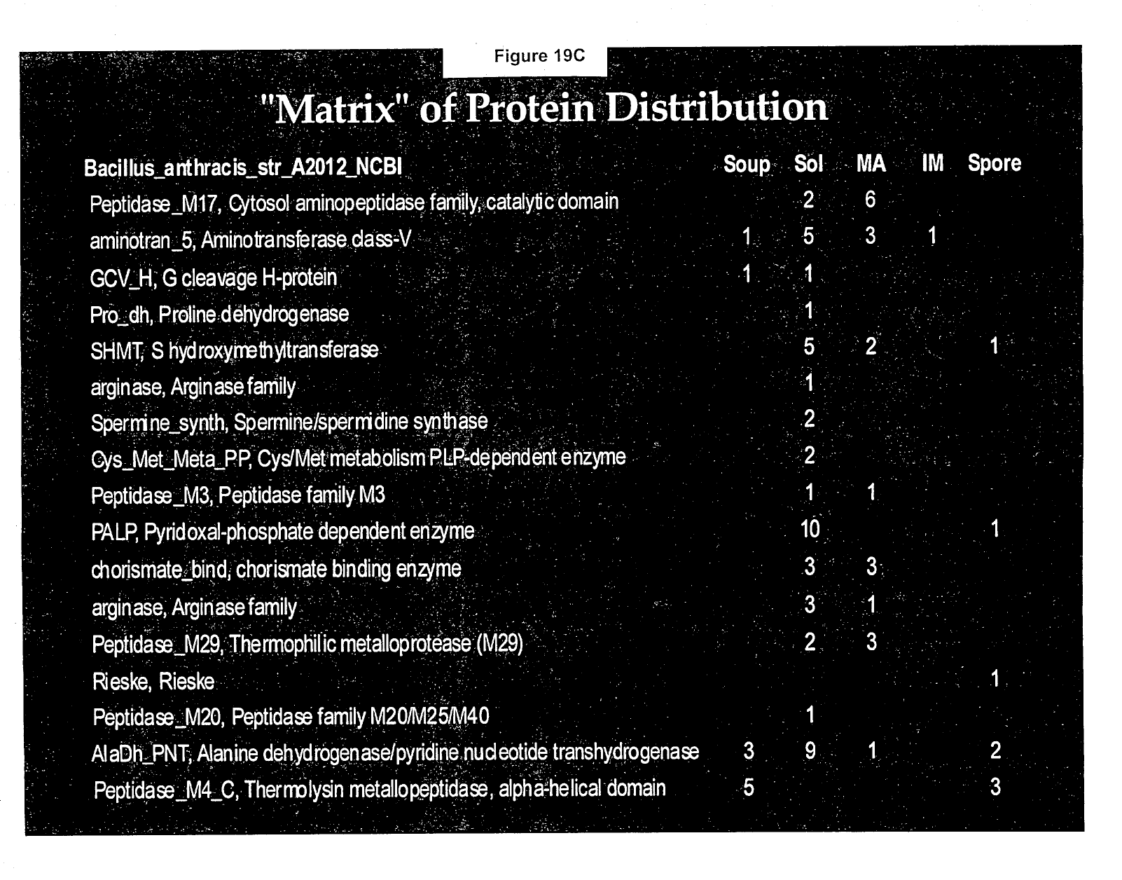

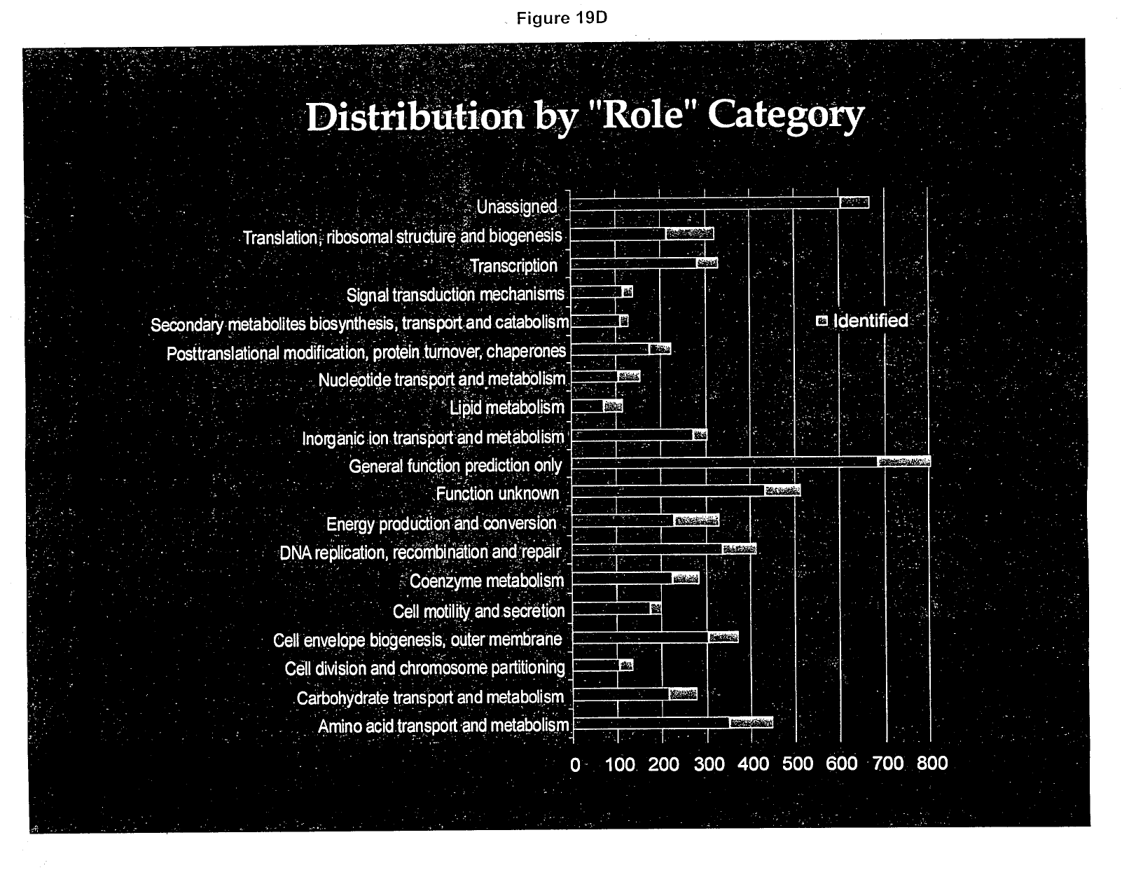

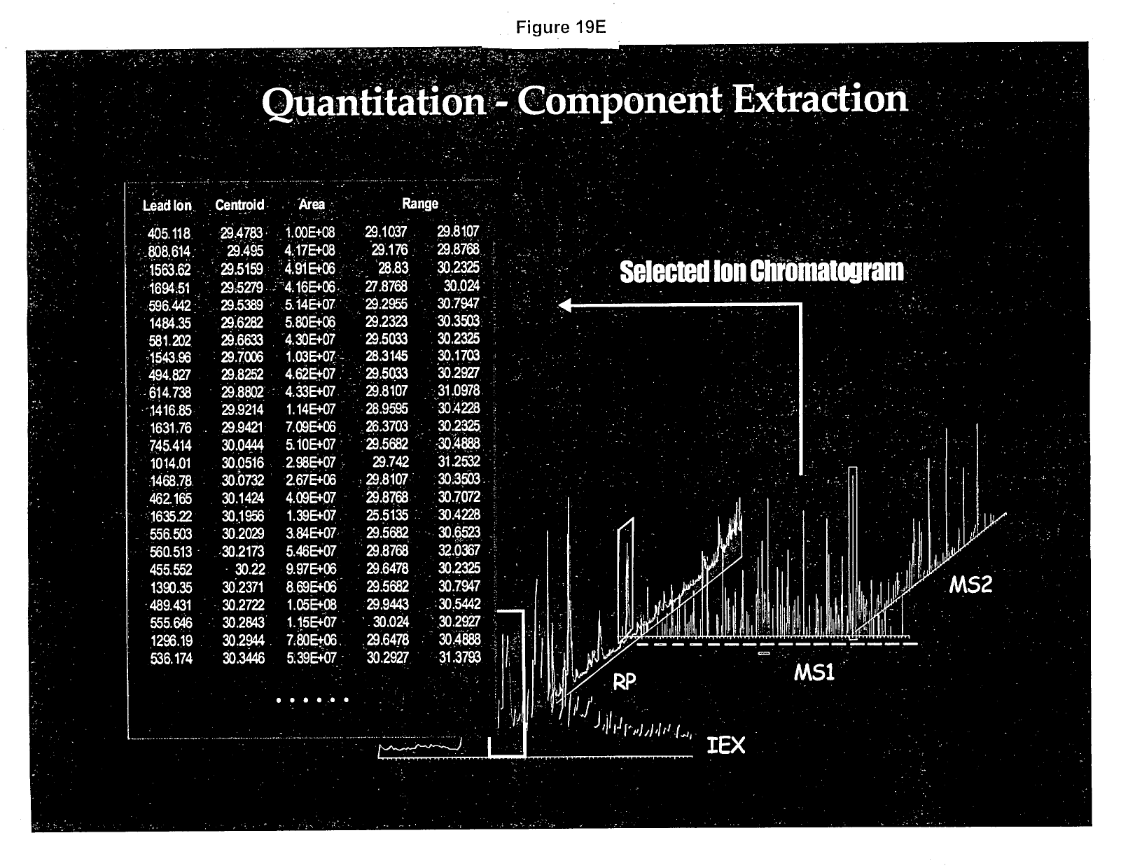

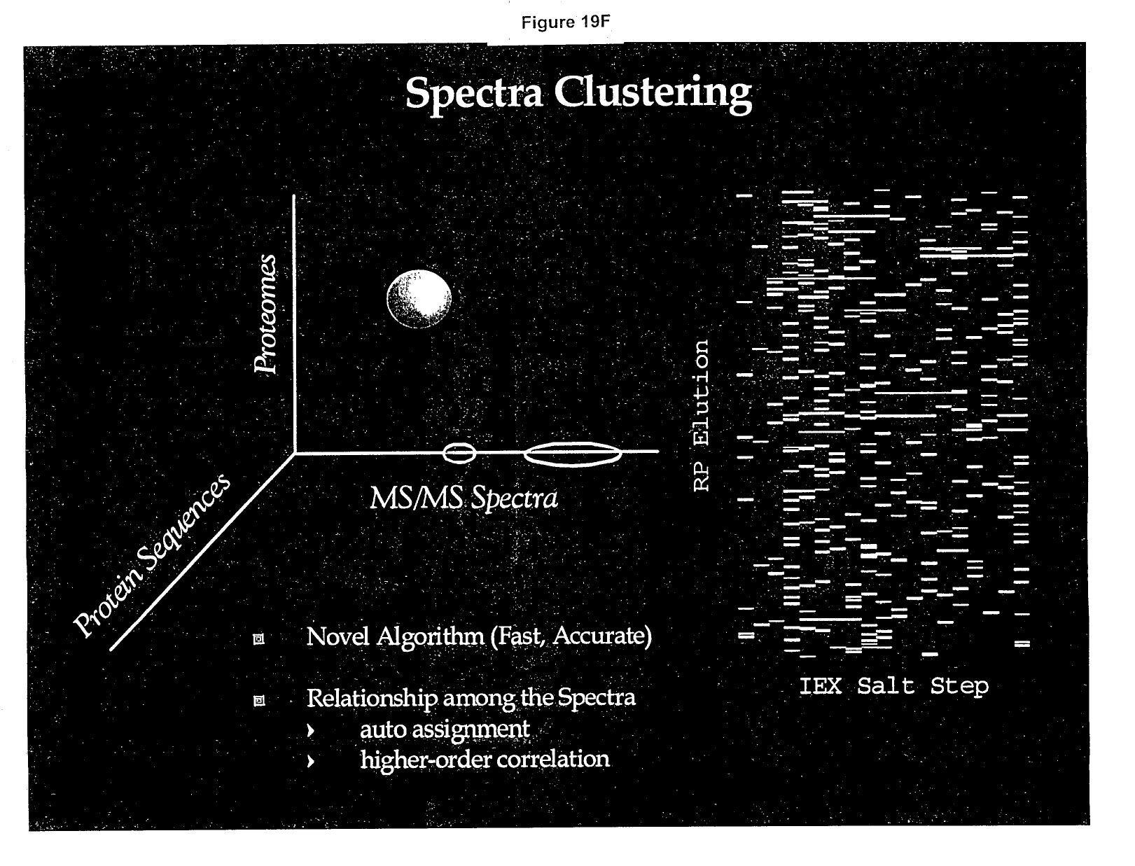

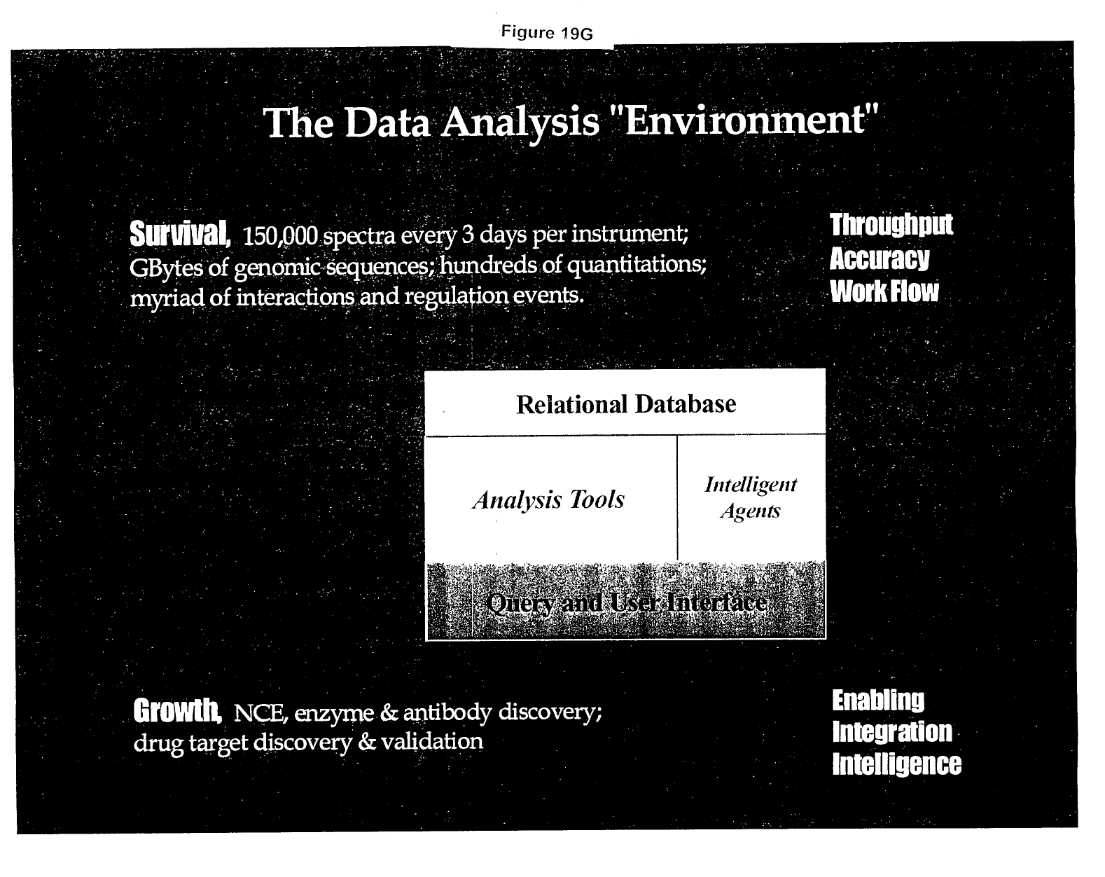

This above described exemplary data analysis methods and interpretation of LC-MS or LC-LC-MS quantitative proteomics data are illustrated in FIGS. 19A through 19G.

The exemplary method can effectively extract quantitative information about the peptides from the LC-MS or LC-LC-MS data. This “components” list is largely free of noise and artifacts. A spectra comparison algorithm can specifically identify equivalent spectra. It can apply to any mass spectra including MS and MS/MS spectra. Using the systems and methods of the invention, the reconstruction of the differentially labeled peptides employing the combination of predicted elution and mass values can be effective and comprehensive.

Differential Labeling of Proteins with Fluorescent Dyes

The invention provides methods for the differential labeling of proteins with fluorescent dyes and the subsequent separation and sorting for sequence analysis using multi-dimensional liquid chromatography systems. This aspect of the invention will permit the direct quantitative comparison of two or more complex protein samples with the help of a multi-dimensional column system and fluorescence detection system.

In one aspect, the invention provides a system comprising a platform and fluorescent dyes. The dyes can form covalent bonds with amines in peptides and proteins. The invention uses a multi-dimensional liquid chromatography system to resolve complex mixtures of proteins. The system can be coupled to a fluorescent detector to detect differentially labeled protein species. In one aspect, this platform is miniaturized and fully automated.

In one aspect the invention provides a liquid-phase chromatographic method and protein label approach to allow the direct comparison and sorting of multiplexed protein samples. In one aspect, up to three (or more) complex protein samples are differentially labeled with a dye, e.g., a Cy dye (e.g., either Cy2, Cy3 and/or Cy5 (Cy dyes are described, e.g., in U.S. Pat. Nos. 5,268,486; 5,569,587; 5,627,027), mixed and separated on several subsequent focusing and chromatography columns. Given that all three Cy-dyes have identical charges and purification properties, proteins tagged with these dyes should exhibit similar purification properties. Labeled proteome mixes are applied onto a liquid column chromatography system with several columns coupled in sequence. Possible combinations are: IEF column, followed by strong anion exchange columns coupled to a reverse phase, and other compatible combinations. Protein fractions from e.g. a focusing run can be applied to an ion exchange column. Step elutions can be performed onto the reverse phase column, which can further resolve these fractions. This step-elution/reverse phase procedure can be repeated for each isoelectric focusing procedure. Eluting protein can be routed into a fluorescent detector. Fluorescence emission can be monitored at all Cy-dye wavelengths. Software will detect differential concentrations between eluting peaks and activate a fraction collector for differentially expressed protein peaks. These fractions can then be further analyzed by mass spectrometer based detection techniques. In alternative aspects, the invention provides multiplexed column systems, automation and/or miniaturization of these systems and methods.

The systems and methods of the invention enhances the quality, sensitivity and throughput of differential proteomics. Unlike conventional electrophoretic approaches, e.g., 2-D electrophoresis, see, e.g., U.S. Pat. Nos. 6,136,173; 6,127,134 (differential 2-D electrophoresis); 6,064,754 (differential 2-D electrophoresis), the method of the invention is highly reproducible, can analyze entire proteomes and can be coupled to automated sample collection devices or proteomic analysis instrumentation. The method of the invention allows the separation of all solubilized proteins in liquid phase and may avoid surface effects commonly associated with some separations, e.g., as described in U.S. Pat. No. 6,013,165 (e.g., PROTEINPROFILER™ separations).

The multi-dimensional column systems and corresponding methods of invention enhance the separation of very complex samples. By using the fluorescence labeling systems and corresponding methods of invention, pooling of differential samples is possible to allow for direct comparisons. The systems and methods of the invention are highly sensitive because fluorescence detection is currently one of the most sensitive forms of detection.

The invention detects differences in protein concentration in two or more samples. It combines the differential labeling of proteins with cyanine dyes or the like, with existing chromatographic protein separation techniques. The methods and systems of the invention comprise use an FPLC system and/or HPLC system with appropriate fluorescence detectors to detect differential protein species and sort them into fractions. The invention provides a sensitive pre-fractionation of protein samples that are differentially expressed. This method can be used instead of ICAT.

### EXAMPLES

The following examples are offered to illustrate, but not to limit the claimed invention.

### Example 1

Metabolic Flux Analysis (MFA)

The following example describes implementation of an exemplary Metabolic Flux Analysis (MFA), which is applied in the real time analysis of cell cultures in the methods of the invention. FIG. 2 shows one example of the processing steps that may be implemented by a computer program.

Metabolic Flux Analysis (MFA) is important analysis technique of metabolic engineering. A flux balance can be written for each metabolite (yi) within a metabolic system to yield the dynamic mass balance equations that interconnect the various metabolites. Generally, for a metabolic network that contains m compounds and n metabolic fluxes, all the transient material balances can be represented by a single matrix equation:

dY/dt=A X(t)−r(t)

where

Y: m dimensional vector of metabolite amounts per cell

X: n metabolic fluxes

A: Stoichiometric m x n matrix, and

r: vector of specific rates from measurements

The time constants characterizing metabolic transients are typically very rapid compared to the time constants of cell growth and process dynamics, therefore, the mass balances can be simplified to only consider the steady-state behavior. Eliminating the derivative yields: A X(t)=r(t) .

Provided that m>=n and A is full rank, the weighted least squares solution of the above equation is: X=(A^{T}A)⁻¹A^{T}r .

The sensitivity of the solution can be investigated by the matrix: dX/dr=(A^{T}A)⁻¹A^{T}.

The elements of the above matrix are useful for the determination of the change of individual fluxes with respect to the error or perturbation in the measurements.

Inputs

Stoichiometric Equations

A stoichiometry matrix is derived from the chemical equations to be used in the analysis. The matrix consists of coefficients of chemical species involved in the reactions. Rows represent the species and columns represent the equations. For instance, if we consider the equations of energy production in cells:

2 NADH+O2+6 ADP→2 NAD+2 H2O+6 ATP

2 FADH+O2+4 ADP→2 FAD+2 H2O+4 ATP

ATP→ADP

This system yields a stoichiometry matrix with 3 columns and as many rows as species to be considered in the overall system.

| | | | |
|---|---|---|---|
| NADH | −2 | 0 | 0 |
| O2 | −1 | −1 | 0 |
| NAD | 2 | 0 | 0 |
| H2O | 2 | 2 | 0 |
| FADH | 0 | −2 | 0 |
| FAD | 0 | 2 | 0 |
| ATP | 6 | 4 | −1 |
| ADP | −6 | −4 | 1 |

In this case, 8 species are considered so the matrix is 3×8.

Using these templates, the stoichiometric matrix is 35×33, and it is in the EXCEL 97™ file “stoichiex.xls”. This is the matrix ‘A’ described above, and it is derived from the 33 chemical equations below.

1. CENTRAL METABOLIC PATHWAYS

```
  1) GLC + ATP + NAD → 2 PYR + ADP + NADH + H2O
  2) PYR + NADH → LAC + NAD
  3) PYR + NAD → ACCOA + CO2 + NADH
  4) ACCOA + OAA + NAD + H2O → AKG + CO2 + NADH
  5) AKG + NAD → SUCCOA + CO2 + NADH
  6) SUCCOA + ADP + H2O + FAD → FUM + ATP + FADH
  7) FUM + H2O → MAL
  8) MAL + NAD → OAA + NADH
  9) GLN + ADP → GLU + NH3 + ATP
10) GLU + NAD → AKG + NH3 + NADH
11) MAL → PYR + CO2
```

2. BIOMASS SYNTHESIS: C50.5% H8.31% O32.93% N8.26%

12) 0.1016 GLC + 0.031 GLN + 0.008 ARG + 0.0003 ASN + 0.001 GLU +

0.0038 GLY + 0.0028 HIS + 0.0071 ILE + ).008 LEU + ).0043 LYS + 0.001 MET +
0.0152 THR + ).0051 VAL → BIOMASS
3. AMINO ACID METABOLISM

13) PYR + GLU → ALA + AKG
14) SER → PYR + NH3
15) GLY → SER
16) CYS → PYR + NH3
17) ASP + AKG → OAA + GLU
18) ASN → ASP + NH3
19) HIS → GLU + NH3
20) ARG + AKG → GLU
21) PRO → GLU
22) ILE + AKG → SUCCOA + ACCOA + GLU
23) VAL + AKG → GLU + CO2 + SUCCOA
24) MET → SUCCOA
25) THR → SUCCOA + NH3
26) PHE → TYR
27) TYR + AKG → GLU + FUM + 2 ACCOA
28) LYS + 2 AKG → 2 GLU + 2 CO2 + 2 ACCOA
29) LEU + AKG → GLU + 3 ACCOA

4. ANTIBODY FORMATION:

30) 1.05 ARG + 1.98 ASN + 1.96 ASP + 1.42 GLU + 1.31 GLY + 1.59 ILE + 3.79

LEU + 1.97 LYS + 0.67 MET + 0.95 PHE + 5.72 SER 1.32 THR 5.05 TYR + 2.68 VAL →
Ab
5. ENERGY PRODUCTION:

31) 2 NADH + O2 + 6 ADP → 2 NAD + 2 H2O + 6 ATP
32) 2 FADH + O2 + 4 APP → 2 FAD + 2 H2O + 4 ATP

33) ATP → ADP

In order to use this matrix with other mathematics software, it must be converted to a text file. Highlight only the cells that contain numbers, select copy from the Edit menu, and paste into a notepad (or simple text editor) document, e.g., the “Notepad” text editor program that comes with Microsoft Windows™ 3.11, 95 and NT. The file can be saved in a notepad as a text file “*.txt”.

Specific Uptake Rates

The specific uptake rates are calculated from data from a cell culture reactor. This data should also be in a text file as a vector of rates, r, that correspond to the appropriate chemical species, i.e. the rows in the stoichiometry matrix above. In the provided templates, the specific rates are listed in the EXCEL 97™ file “ratex.xls” as well as a text file (exported from Excel) “rate.txt”.

MFA Calculations

With the inputs in the desired form, it is now time to use a mathematics software package to calculate the estimated internal fluxes. This software should be able to handle matrix math and differential equations. One template was made in MATHEMATICA™ 3.0 and is named “mfamath.nb”. The following section assumes that the calculations are done in MATHEMATICA™ 3.0, but the general procedure can be applied with any suitable package.

Read in Data

First the default directory is set using the SetDirectory command:

example: SetDirectory[“a:\mfa\”]

The data is then read in and saved into the A matrix (for the stoichiometry matrix) and the r vector (for the specific rates).

example: A=ReadList[“stoichi.txt, Number, RecordLists -->

True]

r = ReadList[“rate.txt, Number, RecordLists --> True]

Sensitivity Analysis

Next, the sensitivity matrix (dX/dr) is calculated as (A^{T}A)⁻¹A^{T}.

example: sens = Inverse[Transpose[A].A].Transpose[A]

Solution and Error Analysis

The least squares estimation of the flux distributions, x, and the errors, e, are calculated for the over-determined system of equations.

| | |
|---|---|
| example: | x = sens.r |
| | e = r − A.x |

Output of Results

After calculation of the flux estimations, the results must be written to text files for presentation. In the templates provided, 3 results text files are included. These files are “flux.txt” that contains the x vector, “error.txt” that holds the error vector, and “sensitivity.txt” that contains the sensitivity matrix. An example of creating these text files in MATHEMATICA™ is shown below.

Example: a1 = OpenWrite[“flux.txt”. FormatType -> OutputForm];
Write[a1, TableForm[x, TableSpacing -> {0,1}]]; Close[a1]

Presentation of MFA Results

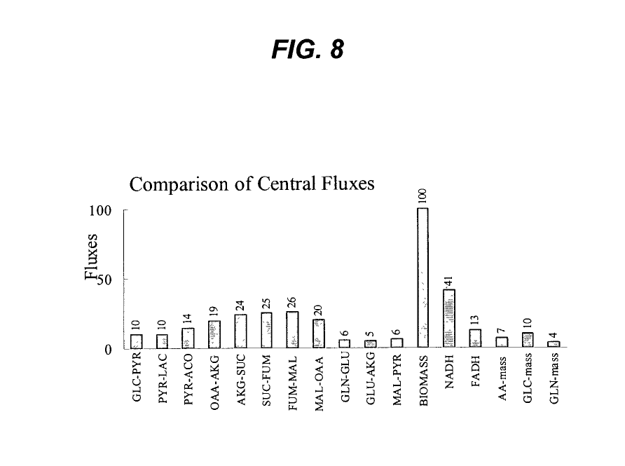

A critical aspect of this analysis is the efficient and clear presentation of the large number of estimated fluxes. The output text files from MATHEMATICA™ can be imported into Excel, and the solution can be plotted as a collection of bar graphs on-a computer display device as shown in FIG. 8.

The EXCEL 97™ file “mfaexc.xls” is the template provided that shows the table of data and the bar graphs for each flux. It also contains a composite bar graph that plots the fluxes together and grouped by metabolic pathway.

An additional way to present the data is to show all the internal fluxes overlain on a map of the relevant metabolic pathways. The POWERPOINT™ template file “mfa.ppt” shows a metabolic map with bar graphs (linked to the Excel file “mfaexc.xls” which must be opened before the file “mfa.ppt”) to show the magnitude of the fluxes. There exists a linking between the Excel file and the POWERPOINT™ presentation. When the data in Excel is updated, the linking in the presentation should be updated.

MATHEMATICA™ and other commercial software tools are used to provide a convenient implementation of the processing steps for real time metabolic flux analysis of this invention. Other software tools may also be used as alternative implementations. One notable example is LABVIEW™ software that has been widely used in data acquisition, data processing, and data presentation in various engineering and scientific applications.

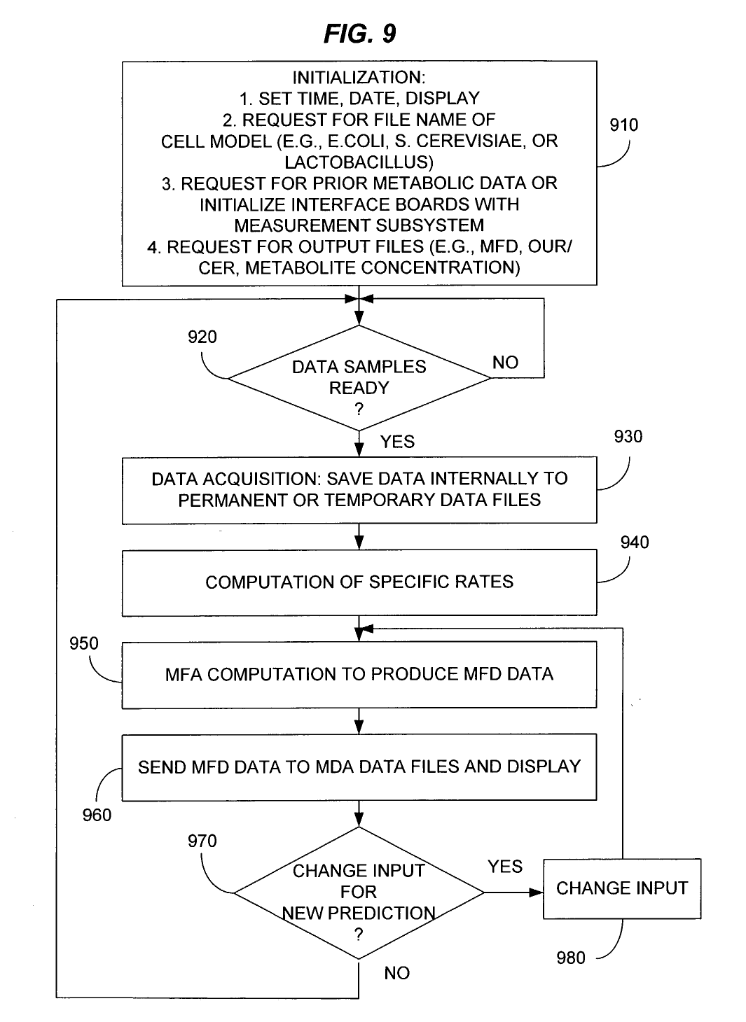

However implemented, the underlying processing steps for MFA computation as described above remain substantially the same. FIG. 9 shows another embodiment of processing steps for real-time MFA-based cell growth and engineering based on the basic operation process in FIG. 2. This operation flow for MFA may be implemented in a computer program by using different software tools based on any suitable programming languages.

Referring to FIG. 9, the process 910 is an initialization process in which the computer initializes various data files and interfaces that are needed for data acquisition, data processing and data output operations in the MFA. For example, the time and date may be set and the computer display may be initialized. As another example, the computer may also request for the file name of a file that stores the cell model for a specified cell of interest which is selected by the system operator. This step may be accomplished by specifying a file path in a local storage device of the computer or by directing the computer to fetch the file from an external electronic information source 350 linked via a communication channel to the MFA computer shown in FIGS. 3 and 5. As another example of the initialization in process 910, the computer may also request for names, and locations of output files that receive MFA data, such as the MFD data, data for OUR/CER, and metabolite concentration. Such output files may be generally in the local computer but may also be in another storage device or computer that is linked to the local computer.

Notably, the initialization process 910 may direct the computer to request for prior metabolic data for the selected cell such as in a prediction MFA application which does not require real-time metabolic measurements. Such data may be accessed from a data file in the local storage device or a remote source such as the source 350 in FIGS. 3 and 5. Alternatively, the initialization process 910 may also direct the computer to initialize interface boards that interconnect the computer to the devices in the sensing subsystem as illustrated in FIGS. 3 and 5. Such initialization establishes the communication between the computer and the devices in the sensing subsystem so that the computer is ready to receive data from the sensing subsystem.

Next, the process 920 determines whether the data samples for MFA computation, either prior data stored in some data file or measured data from the sensing subsystem obtained in real time, are ready. If the data samples are ready, the computer is directed to the next processing step 930. Otherwise, the computer is directed to wait until the data samples are ready. Upon completion of step 920, the computer proceeds to acquire data and store the acquired data in either a permanent data file or a temporary data file in step 930. In addition, the computer computes at step 940 the specific rates based on the acquired data either from the sensing subsystem or from a data file. With the cell model and the results of step 940, the computer is directed in step 950 to carry out the computation for the metabolic fluxes from the matrix equation AX=r. The computed X is then sent to MDA data files and the computer display.

For purpose of predicting the metabolic fluxes, the computer may be next directed to ask the operator whether to change the input for a new prediction. If the operator wants to do so, the computer is directed to request for the changed input and, upon receiving the changed input, to repeat the steps 950 and 960 to produce a new MFD results. If the operator does not need a new MFD prediction, the computer is directed back to wait for new data at step 920.

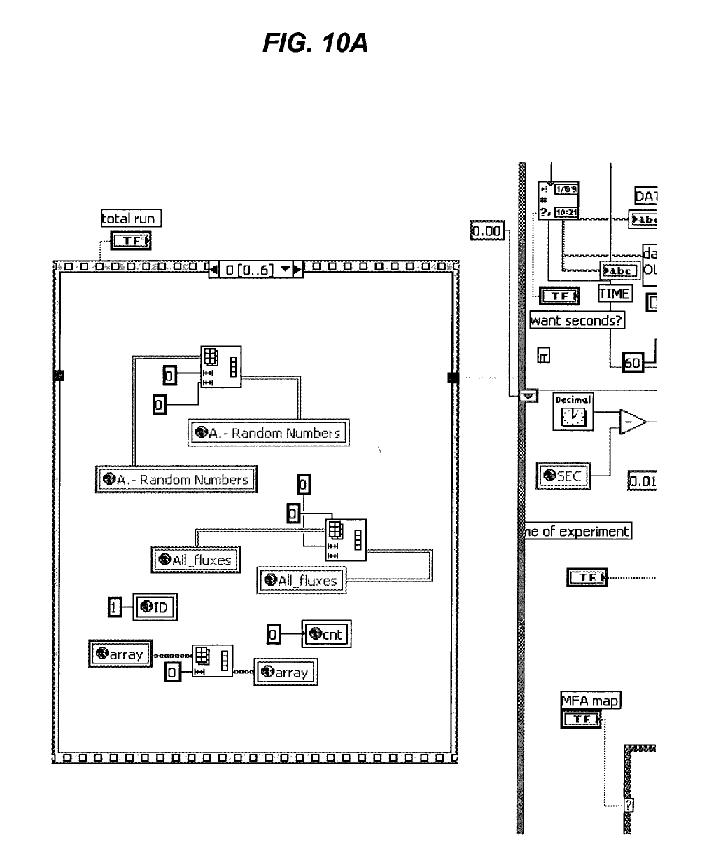

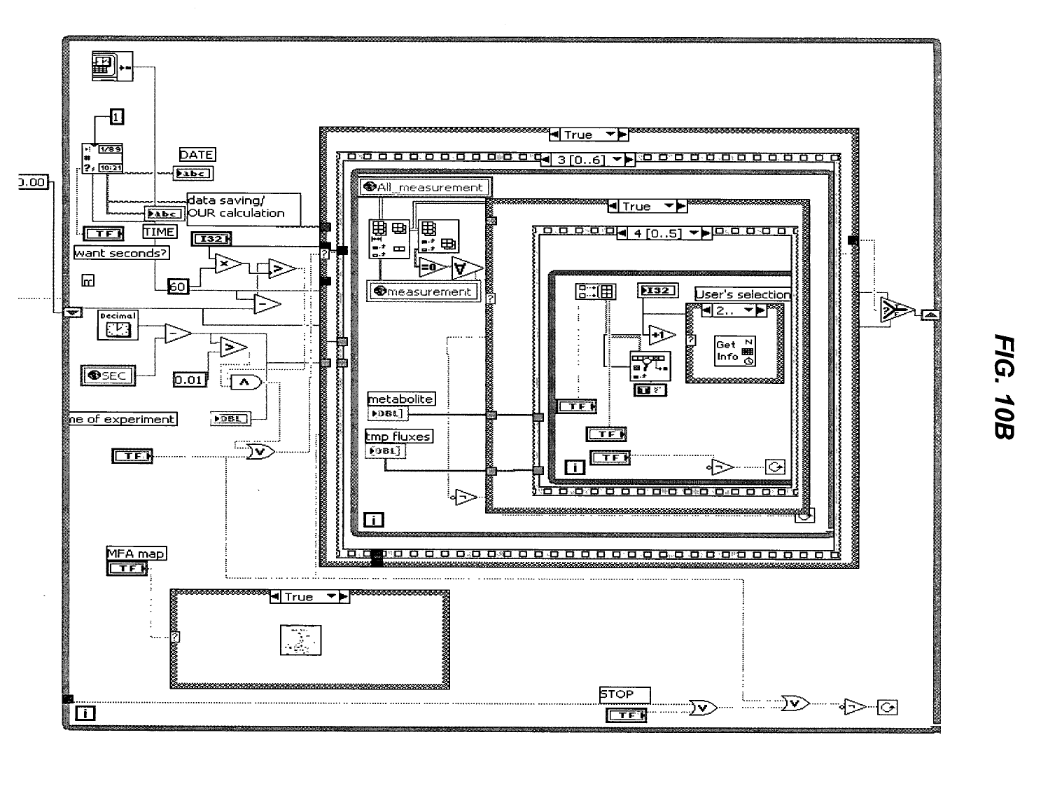

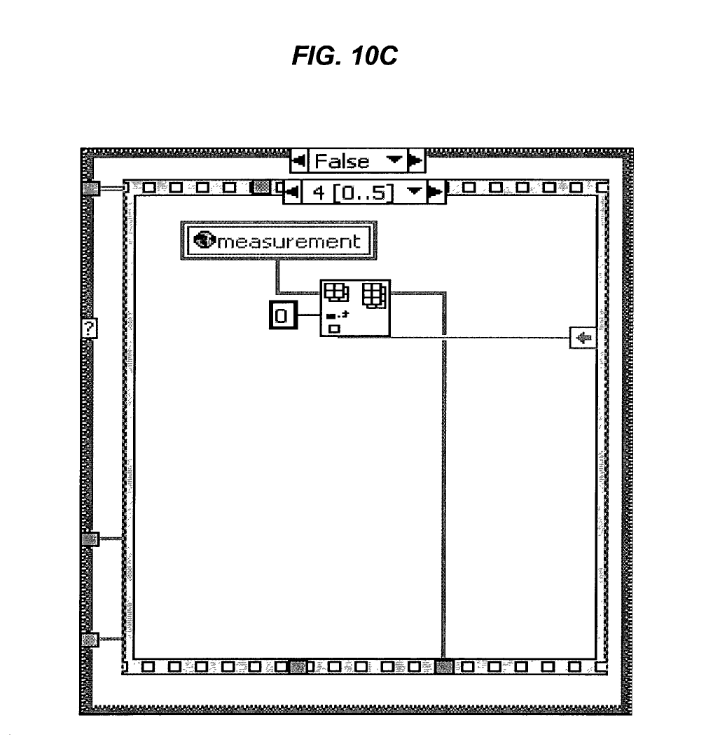

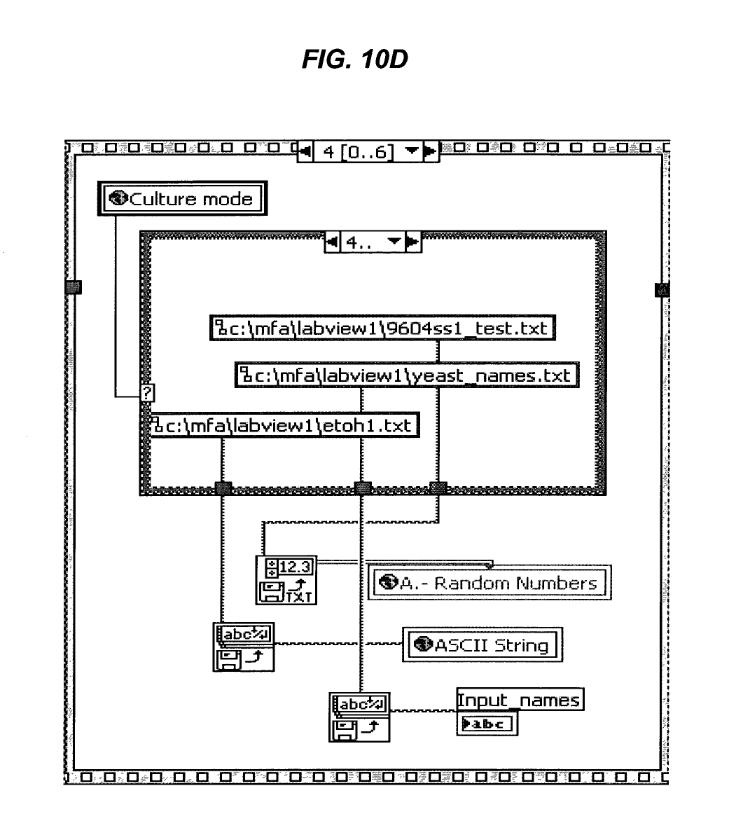

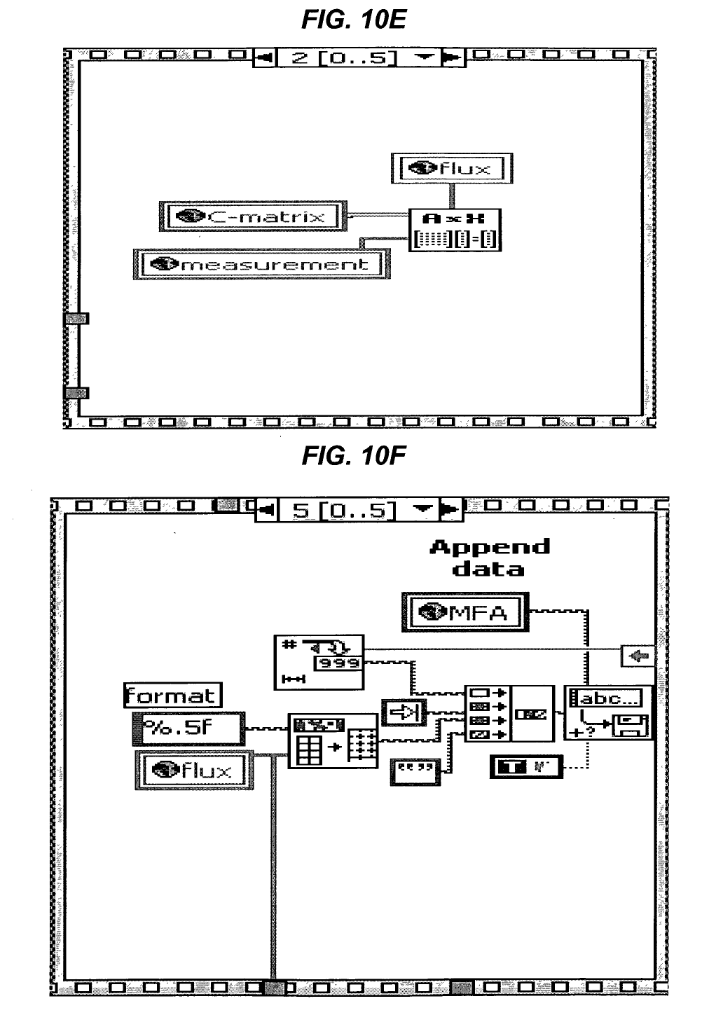

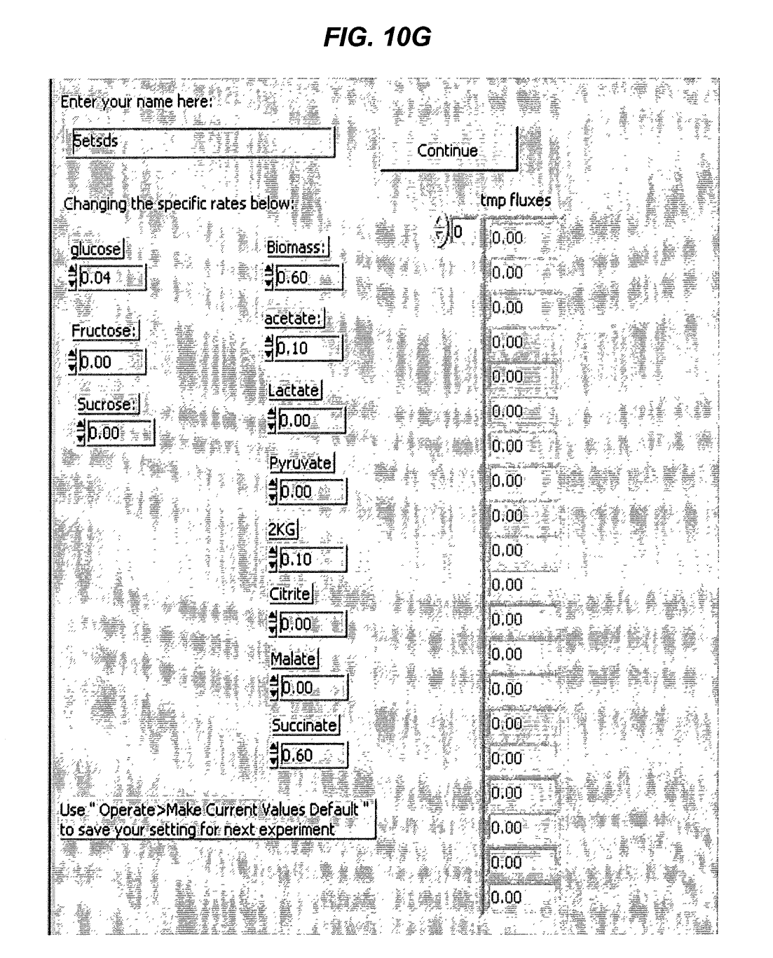

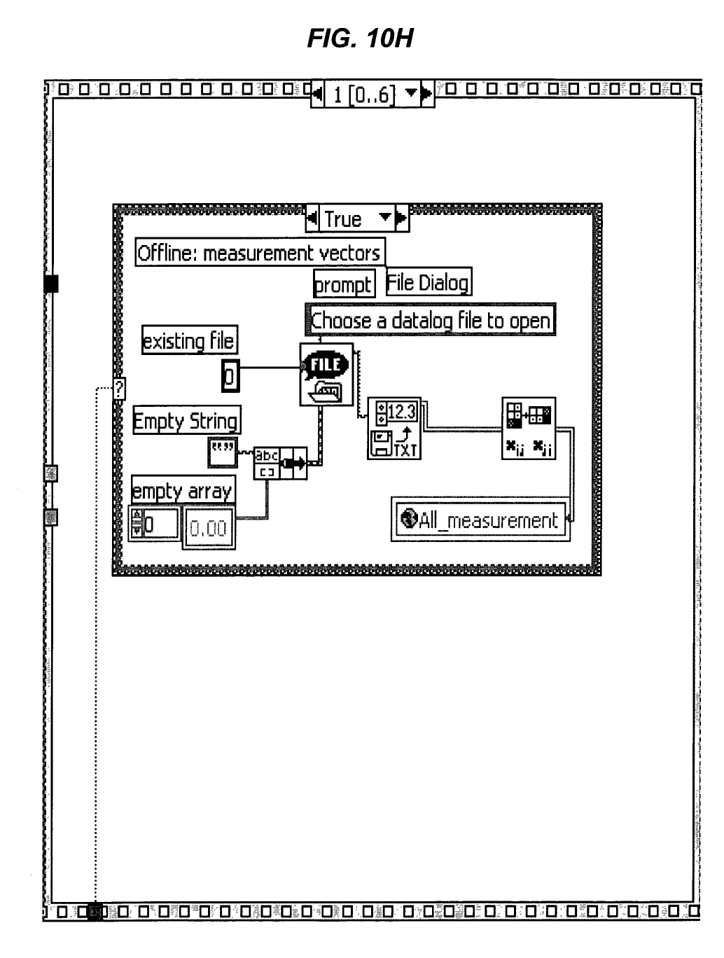

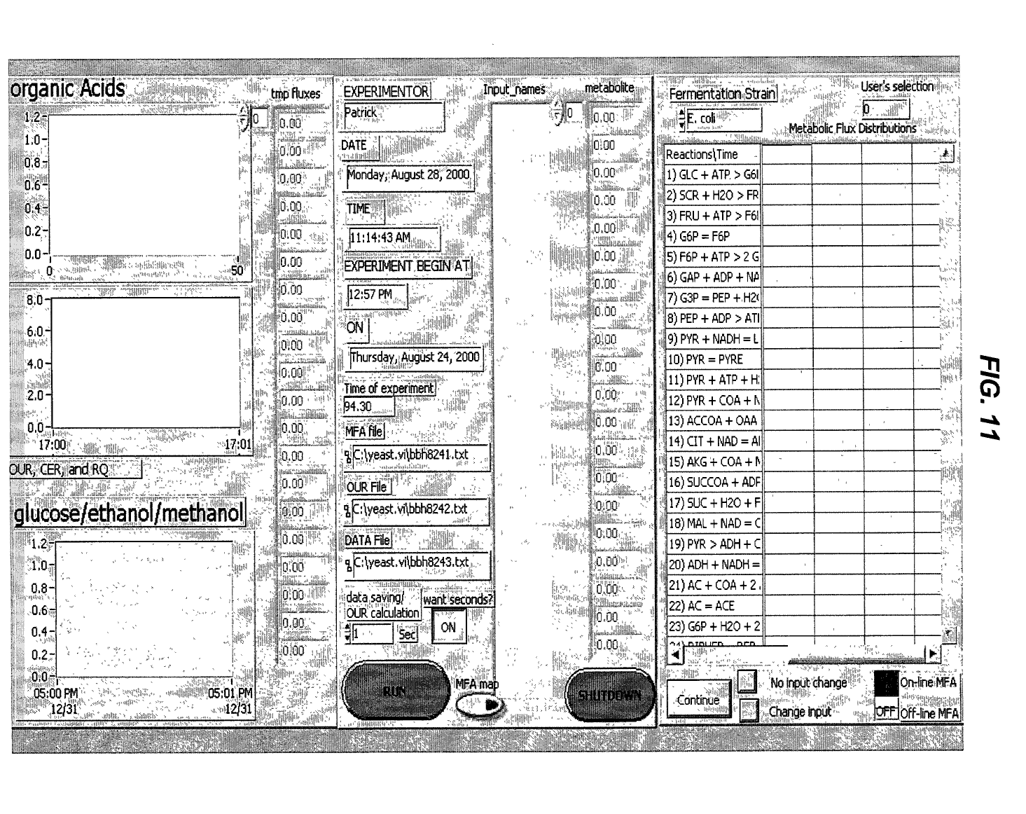

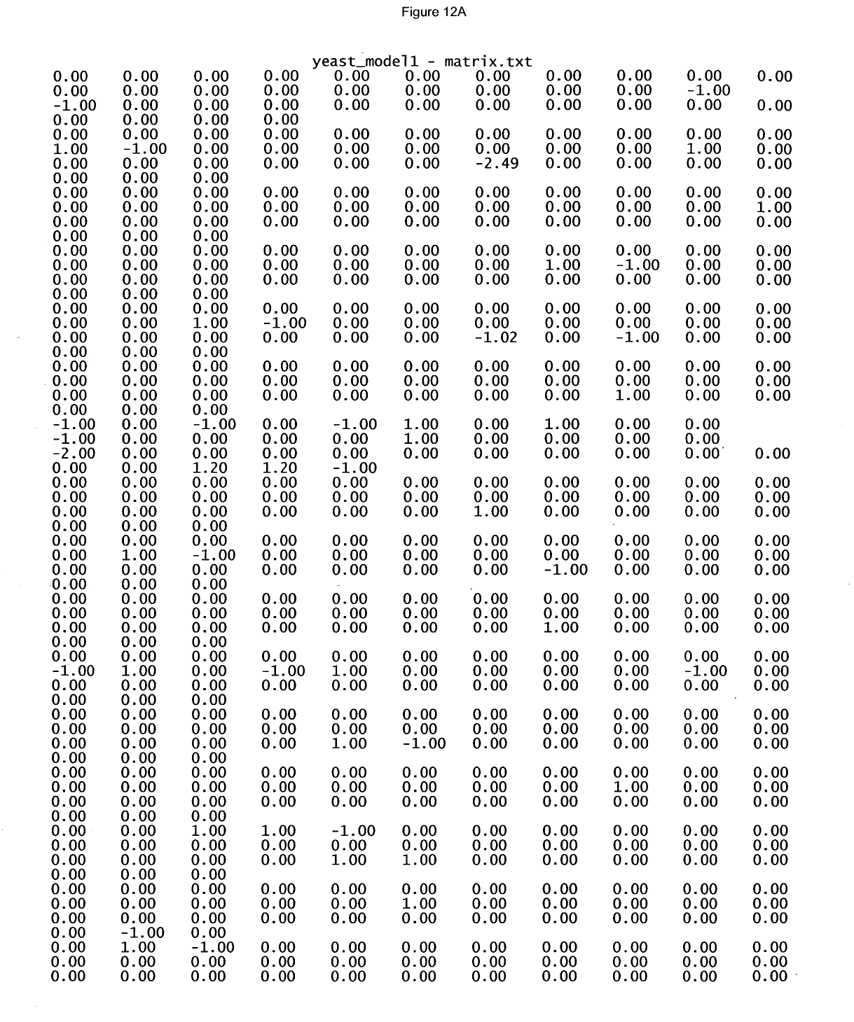

The operations in FIG. 9 may be implemented by using different programming languages. FIGS. 10A through 10H show implementations of the program in FIG. 9 in the user graphical programming form by using the LABVIEW™ software. FIGS. 10A and 10B show exemplary implementations of the steps 910 and 920 in FIG. 9; FIG. 10C shows an exemplary implementation for the step 930 in FIG. 9; FIGS. 10D, 10E, 10F, 10G, and 10H show exemplary implementations of the steps 940, 950, 960, 970, and 980 in FIG. 9; respectively. FIG. 11 shows a display of the LABVIEW™ software for the output from the operations in FIG. 9.

### Example 2

Metabolic Flux Analysis of a Culture of Saccharomyces cerevisiae

The following example describes an exemplary Metabolic Flux Analysis of a culture of the yeast Saccharomyces cerevisiae using the methods of the invention.

Methods and Materials:

Strain and Media:

The yeast Saccharomyces cerevisiae is the most thoroughly investigated eukaryotic model system for the fundamental molecular and genetic study of numerical biological processes (e.g., transcription, translation, cell cycle, membrane transport, etc.) and serves as a widely used biotechnological production organism. Some of the properties that make the yeast Saccharomyces cerevisiae particularly suitable for biological studies include rapid growth, dispersed cells, the ease of replica plating and mutant isolation, a well-defined genetic system, and most important, a highly versatile DNA transformation system. The yeast Saccharomyces cerevisiae Strain ATCC S288C was used in this study. SD medium (Sherman et al., 1986) was used in the experiment. It was made with 0.16% yeast nitrogen base (YNB) without amino acids and hexose (BIO101), 0.5% ammonium sulfate, supplemented with 2% glucose. Cultures were all grown at 30° C. See, e.g., Sherman, F., Fink, G. R., and J. B. Hicks. (1986). Methods in Yeast Genetics. Cold Spring Harbor Laboratory Press, Cold Spring Harbor, N.Y.

For a typical batch experiment, a 15 ml sterile test tube containing 5 ml of SD media was inoculated with a colony from a streaked YPD plate. The yeast culture was grown over night in a shaking incubator (250 rpm) at 30° C. The primary seed was transferred to a 1 L Erlenmeyer shake flask containing 250 ml of pre-warmed SD medium. The culture was grown approximately 12 hours in the same shaking incubator before being used as the secondary seed. The secondary seed was used to inoculate a 5L bioreactor (BIOFLO™ 3000, New Brunswick Scientific Co., Inc. Edison, N.J.).

Fermentation System:

BIOFLO 3000™ has its own controllers for temperature, pH and dissolved oxygen (DO). The S. cerevisaie cultivation process was monitored and controlled automatically using a PENTIUM II™ (233 MHz, Windows 98) equipped with a computer interface board: Analog Input board AT-MIO-16E-10 (National Instruments Corp., Austin, Tex.). The data acquisition and process control program was written in LabVIEW6.0 (National Instruments Corp., Austin, Tex.). The data from bioreactor system, including pH, temperature and dissolved oxygen concentration (DO) are acquired through the AT-MIO-16E-10 board. The compressed air is fed into the bioreactor through a gas flowmeter. The exhaust gas was filtered by putting the tubing into the Drierite bottle (W. A. Hammons Drierite Co., Xenia, Ohio), and then connected to the 1440C O₂ and CO₂ analyzers (Servomex Co., Inc. Norwood, Mass.). The analog outputs of the analyzers are connected to the data acquisition board AT-MIO-16E-10. The temperature was controlled using a circulating water bath (Haake, Berlin, Germany) with a temperature control module.

Analytical Procedures:

During the cultivation period, samples were taken periodically for off-line analysis. Aliquots at 2 mL volumes were withdrawn rapidly from the fermentor, minimizing perturbations to their environment. The samples were then used to determine cell, glucose, ethanol, acetate and organic acid concentrations. Cellular growth was monitored by measuring the optical density (OD) at 600 nm and 660 mn with DU 7400 Spectrophotometer (Beckman Coulter Inc., Fullerton, Calif.). Concentrations of glucose and ethanol were determined using YSI 2700 SELECT BIOCHEMISTRY™ analyzer (YSI Inc., Yellowstone, Ohio). The concentrations of other metabolites in the culture media were determined by HPLC (Rainin Instruments Co. Inc., Woburn, Mass.). An aminex HPX-87H™ ion exchange carbohydrate-organic acid column (Bio-Rad Laboratories, Hercules, Calif.) (@ 65° C. was used with degassed 5 mM sulfuric acid as the mobile phase and UV detection.

Analysis of MFA

The yeast enzymatic reactions used to determine A, the stoichiometry matrix are:

GLC+ATP>G6P+ADP 1)

SCR+H2O>FRU 2)

FRU+ATP>F6P+ADP 3)

G6P=F6P 4)

F6P+ATP>2 GAP+ADP 5)

GAP+ADP+NAD>ADH+G3P+ATP 6)

G3P=PEP+H2O 7)

PEP+ADP>ATP+PYR 8)

PYR+NADH=LAC+NAD 9)

PYR=PYRE 10)

PYR+ATP+H2O+CO2>ADP+OAA 11)

PYR+COA+NAD>ACCOA+CO2+NADH 12)

ACCOA+OAA+H2O=CIT+COA 13)

CIT+NAD=AKG+NADH+CO2 14)

AKG+COA+NAD>SUCCOA+CO2+NADH 15)

SUCCOA+ADP=SUC+COA+ATP 16)

SUC+H2O+FAD=MAL+FADH 17)

MAL+NAD=OAA+NADH 18)

PYR>ADH+CO2 19)

ADH+NADH=ETH+NAD 20)

AC+COA+2 ATP+H2O>ACCOA+2 ADP 21)

AC=ACE 22)

G6P+H2O+2 NADP>RIBU5P+CO2+2 NADPH 23)

RIBU5P=R5P 24)

RIBU5P=X5P 25)

X5P+R5P=S7P+GAP 26)

S7P+GAP=F6P+E4P 27)

X5P+E4P=F6P+GAP 28)

0.934 G6P+0.379 R5P+0.091 GAP+0.650 G3P+0.5 PEP+1.756 PYR+0.951 OAA+1.019 AKG+2.489 ACCOA+11.418 NADPH+1.572 NAD=BIOMAS+1.572 NADH+1.271 CO2+11.418 NADP 29)

CIT=CITE 30)

AKG=AKGE 31)

SUC=SUCE 32)

MAL=MALE 33)

NADH+0.5 O2+1.2 ADP>H2O+1.2 ATP+NAD 34)

FADH+0.5 O2+1.2 ADP>H2O+1.2 ATP+FAD 35)

ATP+H2O>ADP 36)

The measurements of these S. cerevisaie enzymatic reactions taken at 4, 10, 17 and 32 hours of culture to determine X, the metabolic flux distributions are:

| | | hour 4 | hour 10 | hour 17 | hour 32 |
|---|---|---|---|---|---|
| AC | Acetate | 0 | 0 | 0 | 0 |
| ACCOA | Acetyl coenzyme A | 0 | 0 | 0 | 0 |
| ACE | Acetate_out | 0.0274 | −0.0184 | 0.1504 | −0.1124 |
| ADH | alcohol dehydrogenase | 0 | 0 | 0 | 0 |
| AKG | a-Ketoglutarate | 0 | 0 | 0 | 0 |
| AKGE | a-ketoglutarate_out | −0.001 | −0.0102 | 0.0227 | −0.0011 |
| ATP | Adenosine 5-triphosphate | 0 | 0 | 0 | 0 |
| BIOMAS | BIOMASS | 0.246 | 1.18 | 5.3 | 0.035 |
| CIT | Citrate | 0 | 0 | 0 | 0 |
| CITE | Citrate_out | 0.0007 | −0.005 | 0.0155 | 0.0008 |
| COA | Coenzyme A | 0 | 0 | 0 | 0 |
| E4P | Erythrose-4-phosphate | 0 | 0 | 0 | 0 |
| ETH | Ethanol | 2.09 | 24 | −43 | −0.081 |
| F6P | Fructose-6-phosphate | 0 | 0 | 0 | 0 |
| FADH | Flavin adenine dinucleotide, reduced | 0 | 0 | 0 | 0 |
| FRU | Fructose | −0.95 | −3.44 | −7.65 | 0 |
| G3P | 3-phosphoglycerate | 0 | 0 | 0 | 0 |
| G6P | glucose-6-phosphate | 0 | 0 | 0 | 0 |
| GAP | Glyceraldehyde 3-phosphate | 0 | 0 | 0 | 0 |
| GLC | Glucose | −1 | −11.1 | −3.43 | 0 |
| LAC | Lactate | 0.0014 | 0.0025 | 0 | 0 |
| MAL | Malate | 0 | 0 | −0.094 | 0 |
| MALE | Malate_out | 0.0029 | 0.0017 | 0 | 0 |
| NADH | Nicotinamide adenine dinucleotide, | 0 | 0 | 0.0754 | 0.0055 |
| | reduced | | | | |
| NADPH | Nicotinamide adenine dinucleotide | 0 | 0 | 0 | 0 |
| OAA | Oxaloacetate | 0 | 0 | 0 | 0 |
| PEP | Phosphoenol pyruvate | 0 | 0 | 0 | 0 |
| PYR | Pyruvate | 0 | 0 | 0 | 0 |
| PYRE | Pyruvate_out | 0 | 0.0312 | 0 | 0 |
| R5P | ribose 5-phosphate | 0 | 0 | 0.0233 | 0 |
| RIBU5P | ribulose 5-phosphate | 0 | 0 | 0 | 0 |
| S7P | Sedoheptulose-7-phosphate | 0 | 0 | 0 | 0 |
| SCR | Sucrose | 0 | 0 | 0 | 0 |
| SUC | Succinate | 0 | 0 | 0 | 0 |
| SUCCOA | Succinate coenzyme A | 0 | 0 | 0 | 0 |
| SUCE | Sucrose_out | 0.0003 | 0.0027 | 0.0563 | 0.0003 |
| X5P | xylulose-5-phosphate | 0 | 0 | 0 | 0 |

These 4, 10, 17 and 32 hour measurements displayed as a matrix text are:

| 4 | 10 | 17 | 32 |
|---|---|---|---|
| 0.0000 | 0.0000 | 0.0000 | 0.0000 |
| 0.0000 | 0.0000 | 0.0000 | 0.0000 |
| 0.0274 | −0.0184 | 0.1504 | −0.1124 |
| 0.0000 | 0.0000 | 0.0000 | 0.0000 |
| 0.0000 | 0.0000 | 0.0000 | 0.0000 |
| −0.0010 | −0.0102 | 0.0227 | −0.0011 |
| 0.0000 | 0.0000 | 0.0000 | 0.0000 |
| 0.2460 | 1.1800 | 5.3000 | 0.0350 |
| 0.0000 | 0.0000 | 0.0000 | 0.0000 |
| 0.0007 | −0.0050 | 0.0155 | 0.0008 |
| 0.0000 | 0.0000 | 0.0000 | 0.0000 |
| 0.0000 | 0.0000 | 0.0000 | 0.0000 |
| 2.0900 | 24.0000 | −43.0000 | −0.0810 |
| 0.0000 | 0.0000 | 0.0000 | 0.0000 |
| 0.0000 | 0.0000 | 0.0000 | 0.0000 |
| −0.9500 | −3.4400 | −7.6500 | 0.0000 |
| 0.0000 | 0.0000 | 0.0000 | 0.0000 |
| 0.0000 | 0.0000 | 0.0000 | 0.0000 |
| 0.0000 | 0.0000 | 0.0000 | 0.0000 |
| −1.0000 | −11.1000 | −3.4300 | 0.0000 |
| 0.0014 | 0.0025 | 0.0000 | 0.0000 |
| 0.0000 | 0.0000 | −0.0940 | 0.0000 |
| 0.0029 | 0.0017 | 0.0000 | 0.0000 |
| 0.0000 | 0.0000 | 0.0754 | 0.0055 |
| 0.0000 | 0.0000 | 0.0000 | 0.0000 |
| 0.0000 | 0.0000 | 0.0000 | 0.0000 |
| 0.0000 | 0.0000 | 0.0000 | 0.0000 |
| 0.0000 | 0.0000 | 0.0000 | 0.0000 |
| 0.0000 | 0.0312 | 0.0000 | 0.0000 |
| 0.0000 | 0.0000 | 0.0233 | 0.0000 |
| 0.0000 | 0.0000 | 0.0000 | 0.0000 |
| 0.0000 | 0.0000 | 0.0000 | 0.0000 |
| 0.0000 | 0.0000 | 0.0000 | 0.0000 |
| 0.0000 | 0.0000 | 0.0000 | 0.0000 |
| 0.0000 | 0.0000 | 0.0000 | 0.0000 |
| 0.0003 | 0.0027 | 0.0563 | 0.0003 |
| 0.0000 | 0.0000 | 0.0000 | 0.0000 |

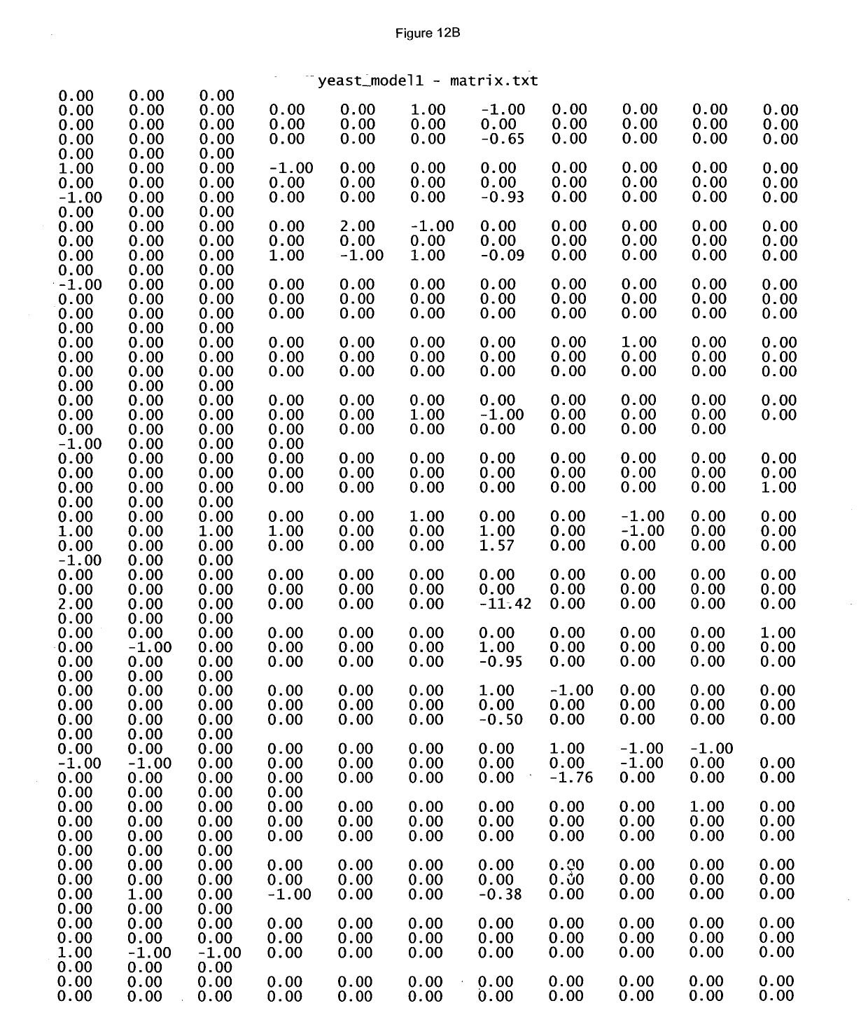

The matrix measurements are shown in FIG. 12, FIGS. 12A (page 1), 12B (page 2) and 12C (page 3).

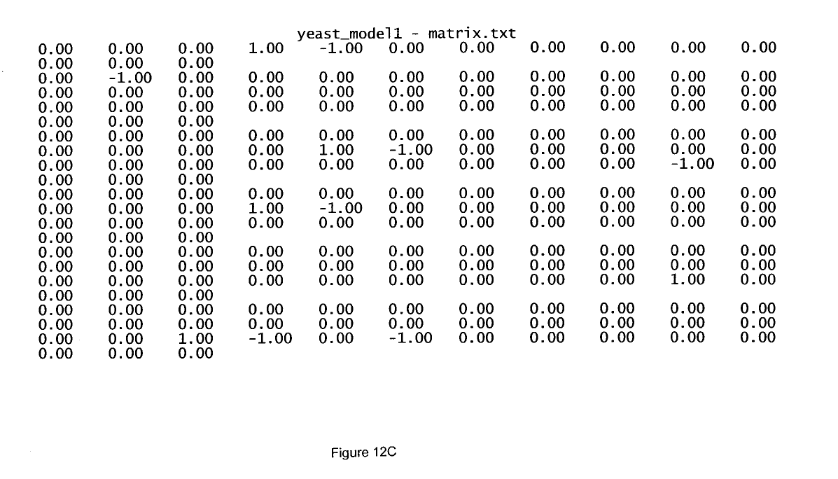

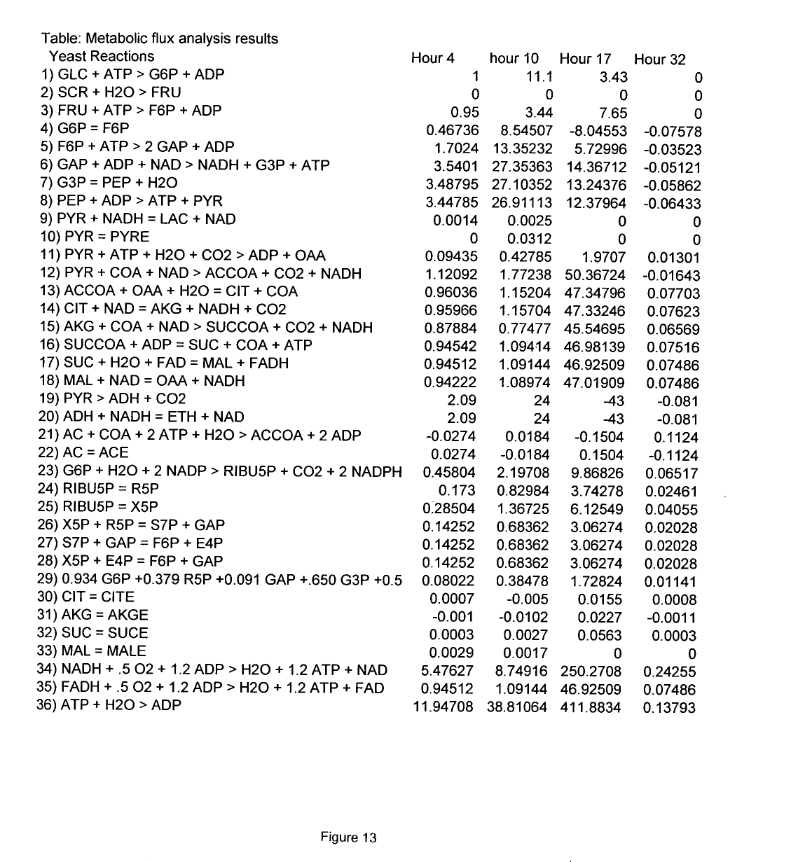

The metabolic flux analysis results for this S. cerevisaie system are shown in Table 1 as FIG. 13.

The system can be summarized as

2.489 ACCOA+11.418 NADPH+1.572 NAD=BIOMAS+1.572 NADH+1.271+CO₂+11.418 NADP

### Example 3

Metabolic Flux Analysis of a Culture of E. Coli

The methods and systems of the invention can be used to determine the metabolic flux analysis for any biological system. Another exemplary MFA determination analyzes an E. coli culture.

The measurements of E. coli enzymatic reactions to determine A, the stoichiometry matrix are:

| | |
|---|---|
| 1) AC | Acetate |
| 2) ACCOA | Acetyl coenzyme A |
| 3) AKG | a-Ketoglutarate |
| 4) ALA | Alanine |
| 5) ASP | aspartate |
| 6) ATP | Adenosine 5-triphosphate |
| 7) BIOMAS | BIOMASS |
| 8) CO2 | |
| 9) E4P | Erythrose-4-phosphate |
| 10) FADH | Flavin adenine-dinucleotide, reduced |
| 11) F6P | Fructose-6-phosphate |
| 12) G3P | Glyceraldehyde 3-phosphate |
| 13) GAP | 3-phosphoglycerate |
| 14) GLC | glucose |
| 15) G6P | glucose-6-phosphate |
| 16) GLUM | glutamate |
| 17) GLUT | Glutamine |
| 18) ISOCIT | isocitrate |
| 19) LAC | Lactate |
| 20) LYSE | Lysine_out |
| 21) LYSI | Lysine |
| 22) MAL | Malate |
| 23) NADH | Nicotinamide adenine dinucleotide, |
| | reduced |
| 24) NADPH | Nicotinamide adenine dinucleotide |
| 25) NH3 | Ammonium |
| 26) | O2 |
| 27) OAA | Oxaloacetate |
| 28) PEP | Phosphoenol pyruvate |
| 29) PYR | Pyruvate |
| 30) RIB5P | ribose 5-phosphate |
| 31) RIBU5P | ribulose 5-phosphate |
| 32) SED7P | Sedoheptulose-7-phosphate |
| 33) SUC | Succinate |
| 34) SUCCOA | Succinate coenzyme A |
| 35) TREHAL | trehalose |
| 36) VAL | valine |
| | xylulose-5- |
| 37) XYL5P | phosphate |

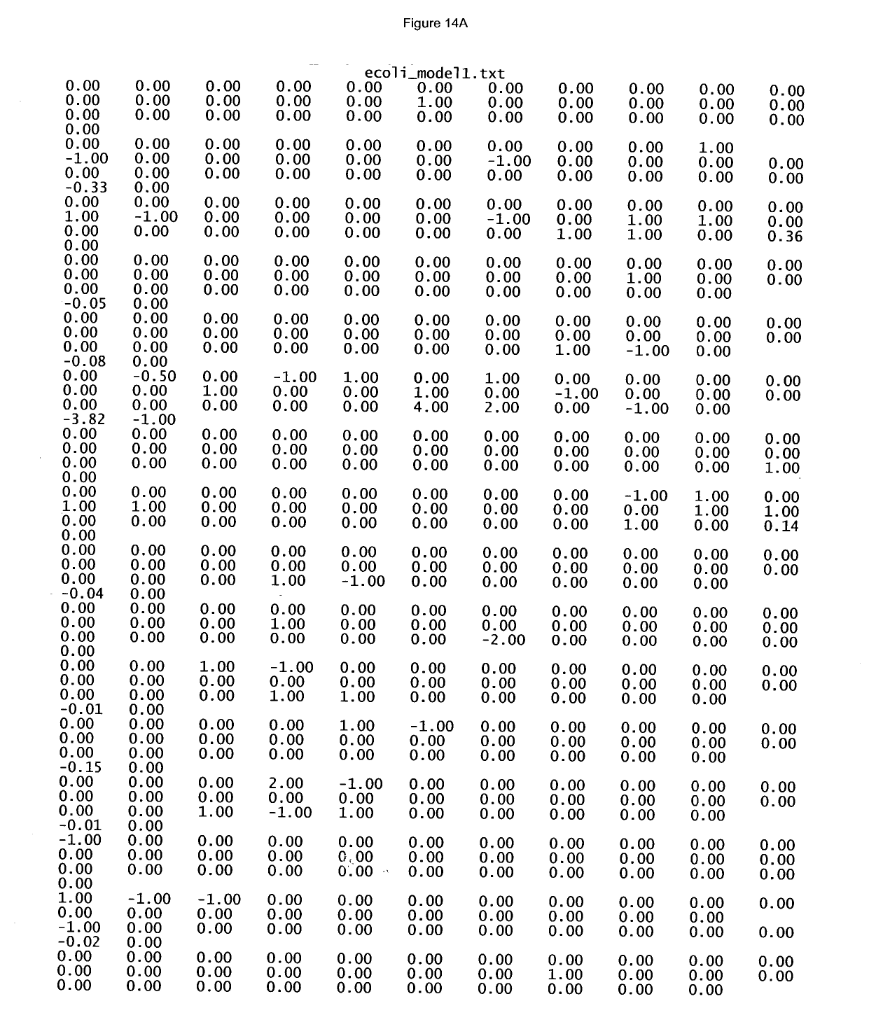

The matrix measurements are shown in FIG. 14, FIGS. 14A (page 1), 14B (page 2) and 14C (page 3).

### Example 4

Identifying Proteins by Differential Labeling of Peptides

An exemplary method for identifying proteins by differential labeling of peptides is provided, as described below.

First, a denatured and reduced protein mixture is digested with trypsin to produce peptide fragments. The mixture is loaded onto a microcapillary column containing a sulfonated styrene resin (e.g., SCX resin, as from Dionex Corporation, Sunnyvale, Calif.) upstream of RPC resin (Rapid Prototyping Chemicals, Switzerland), eluting directly into a tandem mass spectrometer. A discrete fraction of the absorbed peptides are displaced from the SCX column onto the RPC column using a step gradient of salt, causing the peptides to be retained on the RPC column while contaminating salts and buffers are washed through. Peptides are then eluted from the RPC column using an acetonitrile gradient, and analyzed by MS/MS. This process is repeated using increasing salt concentration to displace additional fractions from the SCX column. This is applied in an iterative manner; it can be repeated 10 to 20, or more, times.

The MS/MS data from all of the fractions are analyzed by database searching, as described, for example, by Yates, J. R., III, et al (1995) Anal. Chem. 67, 1426-1436; Eng, J. et al (1994) J. Amer. Mass Spectrom. 5, 976-989. The data are combined to give an overall picture of the protein components present in the initial sample. The MudPIT technique can be run in a fully automated system. The use of two dimensions for chromatographic separation also greatly increases the number of peptides that can be identified from very complex mixtures.

### Example 5

Identifying Proteins by Differential Labeling of Peptides

An exemplary method for synthesizing a differential labeling reagent is provided, as described below.

The invention provides chimeric labeling reagents comprising biotin and an amino acid reactive moiety, such as succimide, isothiocyanate, isocyanate. The amino acid reactive moiety can be attached directly or indirectly (i.e., through a linker) to a biotin, or equivalent.

The biotin can comprise up to 6 deuterium atoms or six hydrogen atoms. Biotin synthesis is described, e.g., in U.S. Pat. No. 4,876,350.

Alternatively, other isotopes, such a ¹³C, ¹⁸O, as described above, can be incorporated either into the biotin moiety, the amino acid reactive moiety or the crosslinker moiety. The biotin facilitates purification, see, e.g., WO 00/11208, and, by comprising at least one isotope, simultaneously allows mass discrimination in the mass spectrometer. The activated group allows covalent bonding to amino acids, such as lysines or cysteines.

An exemplary precursor to biotin that can be used is:

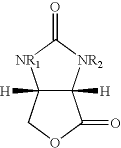

A Grignard reaction is performed with the following compound: XMg—(CD2)4-MgX, where X is chlorine or bromine. The reaction is similar to the one described in U.S. Pat. No. 4,876,350, which describes the chemical synthesis of regular biotin.

A deuteurated and undeuteurated biotin, subsequently derivatized to a pentafluorophenyl ester, can then be attached to iodoacetic acid anhydride or as an NHS ester, or other amino acid reactive groups. For example,

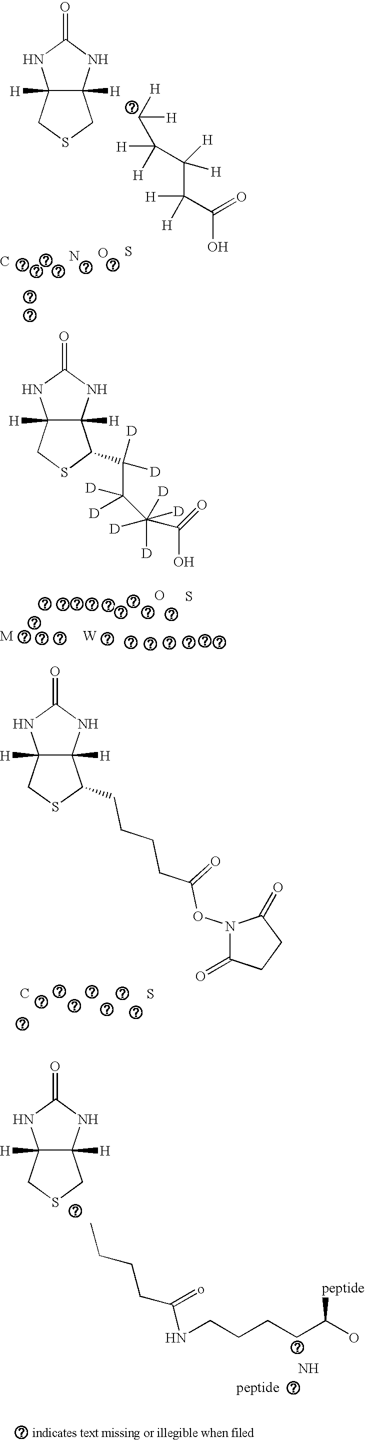

This technology allows the direct comparison between two differential proteome samples. For example, protein samples are differentially tagged with the isotope-coded affinity tags of the invention. These tags are only distinguishable by having different isotope compositions. The isotope- (e.g., deuterium-) containing moiety can be the biotin, the linker or the amino acid reactive group, or any combination thereof. The biotin moiety facilitates purification of the peptides. An isotopically “heavy” and isotopically “light” tagged peptides are separately mixed with denatured differential protein samples. The tagged proteins are digested with a protease before or after mixing of samples. Tagged peptides are purified on an avidin column. The column is washed, and the tagged peptides eluted. After elution of the tagged peptides, the peptide mixture is separated using capillary chromatography and the peptide mass is determined. Peptide masses with the exact difference as the isotopic tag correspond to the identical peptide species and can be directly compared quantitatively.

### Example 5

Lipid Extraction Protocols

An exemplary lipid extraction protocol is described in this example

I. Yeast Preparation.

1. Pick colony from yeast strain and grow overnight @ 24° C. in liquid medium.

2. Obtain two erlenmeyer yeast shaker flasks. Place in each flask: 50 mL YPD media and 4 mL of yeast sample.

3. Place one flask on shaker @ 24° C. for 7 hours.

4. Place the other flask on shaker @ 24° C. for 2 hours then move to a 37° C. shaker for an additional 5 hours.

5. Remove both flasks (after a total of seven hours) and centrifuge. Remove YPD media from yeast pellets. Keep frozen 'til ready to use (−20° C.).

II. Lipid Extraction.

1. Resuspend yeast pellets with 1 mL HPLC grade water.

2. Transfer suspension into borosilicate glass culture tubes.

Keep everything on ice. Work in fumehood.

Use all glassware (no plastics!); prior to using all glassware (eg. graduate cylinder): wash with methanol ×3

then with chloroform ×3

When using HPLC-grade solvents, always pour solvent into clean glass container (except water, which can be stored in Falcon tubes).

3. Add in 2 mL of a 1:2 chloroform:methanol solution:

1 part chloroform, 2 parts methanol. Use glass Pasteur pipette.

4. Cover with parafilm and vortex for 1 minute. Avoid the solvent reaching the parafilm. Allow for layers to separate.

5. Centrifuge @ 2000 rpm for 10 minutes.

6. Extract bottom layer into new culture tubes.

7. Add 1 mL HPLC grade water and 1 mL above chloroform-methanol solution.

8. Vortex and centrifuge as before.

9. Extract bottom layer once again into new culture tubes.

10. Dry lipids completely in a stream of nitrogen.

11. Resuspend in chloroform.

### Example 6

Amino Acid Reactive Isotope-Coded Affinity Tags

This example describes an exemplary process to make amino acid reactive isotope-coded affinity tags for use in differential proteomics. In one aspect, the methods use biotins of varying mass to allow simultaneous mass discrimination, e.g., in a mass spectrometer. In one aspect, the invention uses a linkerless ICAT reagent.

In this aspect, the systems and methods of the invention differentially label peptides and proteins with sulfur and amino-group reactive compounds which differ in their isotopic mass. This approach permits the direct quantitative comparison of two or more protein samples with the help of a mass spectrometer. The systems and methods of the invention provide a novel series of compounds, which can form covalent bonds with lysines and cysteines in peptides and proteins.

The systems and methods of the invention provide an approach to make a low molecular weight reagent that can attach to lysines (instead of cysteines, as described, e.g., in isotope tagged compounds in WO 00/11208).

In one aspect, an activated group, such as succimide, isothiocyanate, isocyanate or ON3 is attached to a biotin that either carries two or more, e.g., six (6), deuteriums or two or more, e.g., six (6), hydrogens. The biotin facilitates purification (e.g., as described in WO 00/11208) and simultaneously allows mass discrimination in the mass spectrometer. The activated group allows covalent bonding to amino acids, such as lysines or cysteines. In one aspect, the invention uses a linkerless ICAT reagent.

One skilled in the art will readily appreciate that the present invention is well adapted to carry out the objects and obtain the ends and advantages mentioned as well as those inherent therein. The methods described herein are presently representative of exemplary aspects and are not intended as limitations on the scope of the invention. Changes therein and other uses will occur to those skilled in the art which are encompassed within the spirit of the invention and are defined by the scope of the claims.

What is claimed is:

1. A method for whole cell engineering of new or modified phenotypes by using real-time metabolic flux analysis, the method comprising the following steps:
(a) making a modified cell by modifying the genetic composition of a cell;
(b) culturing the modified cell to generate a plurality of modified cells;
(c) measuring at least one metabolic parameter of the cell by monitoring the cell culture of step (b) in real time; and,
(d) analyzing the data of step (c) to determine if the measured parameter differs from a comparable measurement in an unmodified cell under similar conditions, thereby identifying an engineered phenotype in the cell using real-time metabolic flux analysis.

2. The method of claim 1, wherein the genetic composition of the cell is modified by a method comprising addition of a nucleic acid to the cell.

3. The method of claim 2, wherein the nucleic acid comprises a nucleic acid heterologous to the cell.

4. The method of claim 2, wherein the nucleic acid comprises a nucleic acid homologous to the cell.

5. The method of claim 4, wherein the homologous nucleic acid comprises a modified homologous nucleic acid.

6. The method of claim 5, wherein the homologous nucleic acid comprises a modified homologous gene.

7. The method of claim 1, wherein the genetic composition of the cell is modified by a method comprising deletion of a sequence or modification of a sequence in the cell.

8. The method of claim 1, wherein the genetic composition of the cell is modified by a method comprising modifying or knocking out the expression of a gene.

9. The method of claim 1, further comprising selecting a cell comprising a newly engineered phenotype.

10. The method of claim 9, further comprising culturing the selected cell, thereby generating a new cell strain comprising a newly engineered phenotype.

11. The method of claim 9, wherein the newly engineered phenotype is selected from the group consisting of an increased or decreased expression or amount of a polypeptide, an increased or decreased amount of an mRNA transcript, an increased or decreased expression of a gene, an increased or decreased resistance or sensitivity to a toxin, an increased or decreased resistance use or production of a metabolite, an increased or decreased uptake of a compound by the cell, an increased or decreased rate of metabolism, and an increased or decreased growth rate.

12. The method of claim 1, further comprising isolating a cell comprising a newly engineered phenotype.

13. The method of claim 1, wherein the newly engineered phenotype is a stable phenotype.

14. The method of claim 13, wherein modifying the genetic composition of a cell comprises insertion of a construct into the cell, wherein construct comprises a nucleic acid operably linked to a constitutively active promoter.

15. The method of claim 1, wherein the newly engineered phenotype is an inducible phenotype.

16. The method of claim 15, wherein modifying the genetic 5 composition of a cell comprises insertion of a construct into the cell, wherein construct comprises a nucleic acid operably linked to an inducible promoter.

17. The method of claim 2, wherein nucleic acid added to the cell in step (a) is stably inserted into the genome of the cell.

18. The method of claim 2, wherein nucleic acid added to the cell in step (a) propagates as an episome in the cell.

19. The method of claim 2, wherein nucleic acid added to the cell in step (a) encodes a polypeptide.

20. The method of claim 19, wherein the polypeptide comprises a modified homologous polypeptide.

21. The method of claim 19, wherein the polypeptide comprises a heterologous polypeptide.

22. The method of claim 2, wherein the nucleic acid added to the cell in step (a) encodes a transcript comprising a sequence that is antisense to a homologous transcript.

23. The method of claim 1, wherein modifying the genetic composition of the cell in step (a) comprises increasing or decreasing the expression of an mRNA transcript.

24. The method of claim 1, wherein modifying the genetic composition of the cell in step (a) comprises increasing or decreasing the expression of a polypeptide.

25. The method of claim 1, wherein modifying the homologous gene in step (a) comprises knocking out expression of the homologous gene.

26. The method of claim 1, wherein modifying the homologous gene in step (a) comprises increasing the expression of the homologous gene.

27. The method of claim 1, wherein the heterologous gene in step (a) comprises a sequence-modified homologous gene, wherein the sequence modification is made by a method comprising the following steps:
(a) providing a template polynucleotide, wherein the template polynucleotide comprises a homologous gene of the cell;
(b) providing a plurality of oligonucleotides, wherein each oligonucleotide comprises a sequence homologous to the template polynucleotide, thereby targeting a specific sequence of the template polynucleotide, and a sequence that is a variant of the homologous gene;
(c) generating progeny polynucleotides comprising non-stochastic sequence variations by replicating the template polynucleotide of step (a) with the oligonucleotides of step (b), thereby generating polynucleotides comprising homologous gene sequence variations.

28. The method of claim 1, wherein the heterologous gene in step (a) comprises a sequence-modified homologous gene, wherein the sequence modification is made by a method comprising the following steps:
(a) providing a template polynucleotide, wherein the template polynucleotide comprises sequence encoding a homologous gene;
(b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence;
(c) combining a building block polynucleotide with a template polynucleotide such that the building block polynucleotide cross-over reassembles with the template polynucleotide to generate polynucleotides comprising homologous gene sequence variations.

29. The method of claim 1, wherein the cell is a prokaryotic cell.

30. The method of claim 29, wherein the prokaryotic cell is a bacterial cell.

31. The method of claim 1, wherein the cell is a selected from the group consisting of a fungal cell, a yeast cell, a plant cell and an insect cell.

32. The method of claim 1, wherein the cell is a eukaryotic cell.

33. The method of claim 32, wherein the cell is a mammalian cell.

34. The method of claim 33, wherein the mammalian cell is a human cell.

35. The method of claim 1, wherein the measured metabolic parameter comprises rate of cell growth.

36. The method of claim 35, wherein the rate of cell growth is measured by a change in optical density of the culture.

37. The method of claim 1, wherein the measured metabolic parameter comprises a change in the expression of a polypeptide.

38. The method of claim 37, wherein the change in the expression of the polypeptide is measured by a method selected from the group consisting of a one-dimensional gel electrophoresis, a two-dimensional gel electrophoresis, a tandem mass spectography, an RIA, an ELISA, an immunoprecipitation and a Western blot.

39. The method of claim 1, wherein the measured metabolic parameter comprises a change in expression of at least one transcript, or, the expression of a transcript of a newly introduced gene.

40. The method of claim 39, wherein the change in expression of the transcript is measured by a method selected from the group consisting of a hybridization, a quantitative amplification and a Northern blot.

41. The method of claim 40, wherein transcript expression is measured by hybridization of a sample comprising transcripts of a cell or nucleic acid representative of or complementary to transcripts of a cell by hybridization to immobilized nucleic acids on an array.

42. The method of claim 1, wherein the measured metabolic parameter comprises an increase or a decrease in a secondary metabolite.

43. The method of claim 42, wherein secondary metabolite is glycerol, ethanol, methanol or a combination thereof.

44. The method of claim 1, wherein the measured metabolic parameter comprises an increase or a decrease in an organic acid.

45. The method of claim 44, wherein the organic acid is acetate, butyrate, succinate, oxaloacetate, fumarate, alpha-ketoglutarate, phosphate or a combination thereof.

46. The method of claim 1, wherein the measured metabolic parameter comprises an increase or a decrease in intracellular pH.

47. The method of claim 46, wherein the increase or a decrease in intracellular pH is measured by intracellular application of a dye, and the change in fluorescence of the dye is measured over time.

48. The method of claim 1, wherein the measured metabolic parameter comprises an increase or a decrease in synthesis of DNA over time.

49. The method of claim 48, wherein the increase or a decrease in synthesis of DNA over time is measured by intracellular application of a dye, and the change in fluorescence of the dye is measured over time.

50. The method of claim 1, wherein the measured metabolic parameter comprises an increase or a decrease in uptake of a composition.

51. The method of claim 50, wherein the composition is a metabolite.

52. The method of claim 51, wherein the metabolite is selected from the group consisting of a monosaccharide, a disaccharide, a polysaccharide, a lipid, a nucleic acid, an amino acid and a polypeptide.

53. The method of claim 52, wherein the saccharide, disaccharide or polysaccharide comprises a glucose or a sucrose.

54. The method of claim 50, wherein the composition is selected from the group consisting of an antibiotic, a metal, a steroid and an antibody.

55. The method of claim 1, wherein the measured metabolic parameter comprises an increase or a decrease in the secretion of a by product or a secreted composition of a cell.

56. The method of claim 55, wherein the by product or secreted composition is selected from the group consisting of a toxin, a lymphokine, a polysaccharide, a lipid, a nucleic acid, an amino acid, a polypeptide and an antibody.

57. The method of claim 1, wherein the real time monitoring simultaneously measures a plurality of metabolic parameters.

58. The method of claim 57, wherein real time monitoring of a plurality of metabolic parameters comprises use of a cell growth monitor device.

59. The method of claim 58, wherein the cell growth monitor device is a Wedgewood Technology, Inc., cell growth monitor model 652.

60. The method of claim 58, wherein the real time simultaneous monitoring measures uptake of substrates, levels of intracellular organic acids and levels of intracellular amino acids.

61. The method of claim 57, wherein the real time simultaneous monitoring measures cell density, uptake of glucose; levels of acetate, butyrate, succinate, oxaloacetate, fumarate, alpha-ketoglutarate, phosphate or a combination thereof; levels of intracellular natural amino acids; or a combination thereof.

62. The method of claim 57, further comprising use of a computer-implemented program to real time monitor the change in measured metabolic parameters over time.

63. The method of claim 62, wherein the computer-implemented program comprises a computer-implemented method as set forth in FIG. 1.

64. The method of claim 63, wherein the computer-implemented method comprises metabolic network equations.

65. The method of claim 63, wherein the computer-implemented method comprises a pathway analysis.

66. The method of claim 63, wherein the computer-implemented program comprises a preprocessing unit to filter out the errors for the measurement before the metabolic flux analysis.

67. A method, comprising:
culturing cells in a controllable cell environment;
measuring at least one metabolic parameter to obtain at least two different measurements in real time during the culturing;
processing the two different measurements to determine a rate of change in the metabolic parameter in real time during the culturing; and
using the rate of change in a known metabolic network of the cells to determine a real-time metabolic flux distribution in the cells during the culturing.

68. The method of claim 67, wherein the controllable cell environment comprises a fermentor or a bioreactor.

69. The method of claim 67, wherein the controllable cell environment comprises a flask, a plate, a capillary tube, a test tube, a biomatrix or an artificial organ.

70. The method of claim 67, wherein the controllable cell environment comprises a plurality of microbioreactors.

71. The method of claim 67, wherein a measured metabolic parameter comprises a gas.

72. The method of claim 71, wherein the gas comprises oxygen, methanol or ethanol or a combination thereof.

73. The method of claim 71, wherein the gas is measured by an on-line mass spectrometer.

74. The method of claim 67, wherein a measured metabolic parameter comprises glucose.

75. The method of claim 74, wherein the glucose is measured by an on-line mass spectrometer or bio-analyzer.

76. The method of claim 67, wherein a measured metabolic parameter comprises an organic acid.

77. The method of claim 76, wherein the organic acid comprises acetate, butyrate, succinate, oxaloacetate, fumarate, alpha-ketoglutarate, phosphate or a combination thereof.

78. The method of claim 76, wherein the organic acid is measured by an on-line HPLC.

79. The method of claim 67, further comprising adjusting an operating parameter of the controllable cell environment based on the determined real-time metabolic flux distribution to change the culturing condition to modify the metabolic flux distribution during the culturing.

80. The method of claim 79, wherein the operating parameter is adjusted to direct the metabolic flux distribution towards a desired distribution.

81. The method of claim 79, wherein the operating parameter comprises a substrate supply to the controllable cell environment.

82. The method of claim 79, wherein the metabolic parameter or the operating parameter comprises a temperature of the controllable cell environment.

83. The method of claim 79, wherein the metabolic parameter or the operating parameter comprises an intracellular pH value inside the controllable cell environment.

84. The method of claim 79, wherein the metabolic parameter or the operating parameter comprises a gas exchange rate inside the controllable cell environment for one or more gases produced during the culturing.

85. The method of claim 79, wherein the operating parameter comprises a nutrient supply to the controllable cell environment.

86. The method of claim 79, wherein the operating parameter comprises cell density in the controllable cell environment.

87. The method of claim 86, wherein cell density in the controllable cell environment is monitored by a cell growth monitor device.

88. The method of claim 86, wherein the cells are cultured in a liquid medium and the cell density is monitored by measuring optical density of the cell culture.

89. The method of claim 67, further comprising modifying a genetic composition of one or more initial cells of the cell culture prior to the culturing of step (a).

90. The method of claim 89, wherein the genetic modifying is based on information obtained from a real-time metabolic flux distribution in an initial cell or cell culture, and wherein the real-time metabolic flux distribution is obtained by
measuring a selected metabolic parameter of one initial cell to obtain at least two different measurements in real time during culturing of the initial cell -or cell culture,
processing the two different measurements to determine a rate of change in the selected metabolic parameter in real time, and
using the rate of change in a known initial metabolic network for the initial cell or cell culture to determine the real-time metabolic flux distribution in the initial cell or cell culture.

91. The method of claim 89, wherein the modifying of the genetic composition comprises adding a nucleic acid of an initial cell or cell culture.

92. The method of claim 89, wherein the modifying of the genetic composition comprises altering a nucleic acid of an initial cell or cell culture.

93. The method of claim 89, wherein the modifying of the genetic composition comprises using an optimized directed evolution system to generate evolved chimeric sequences.

94. The method of claim 89, wherein the modifying of the genetic composition comprises knocking out an expression of a selected gene.

95. The method of claim 89, wherein the modifying of the genetic composition further comprises establishing the known metabolic network for the cell or cell culture by using information from at least one of a group consisting of bioinformatics, stoichiometry, microbiology and biochemical engineering knowledge.

96. The method of claim 67, further comprising obtaining information from transcriptome and proteome data of the selected cell; and, combining the information with the real-time metabolic flux distribution in the selected cell to design a metabolic engineering process.

97. The method of claim 67, further comprising providing a computer for processing in real time the two different measurements and determining the real-time metabolic flux distribution in the selected cell during the culturing.

98. The method of claim 97, further comprising using the computer to retrieve information from at least one of a group consisting of bioinformatics, stoichiometry, microbiology, and biochemical engineering knowledge in establishing the known metabolic network for the selected cell.

99. The method of claim 67, wherein the cells are prokaryotic cells.

100. The method of claim 99, wherein the prokaryotic cells are bacterial cells.

101. The method of claim 67, wherein the cells are fungal cells, yeast cells, plant cells or insect cells.

102. The method of claim 67, wherein the cells are eukaryotic cells.

103. The method of claim 102, wherein the cells are mammalian cells.

104. An article comprising a machine-readable medium including machine-executable instructions, the instructions being operative to cause a machine to:
electronically interface with a plurality of measuring devices coupled to a controllable cell environment to, in real time, obtain electronic data indicative of a plurality of metabolic parameters or conditions of cell culturing therein;
process the electronic data, in real time, to produce values for a set of selected metabolic parameters or conditions indicative of real-time metabolic properties of the cultured cells in the controllable cell environment;
retrieve information from at least one database comprising data on a metabolic network for the cultured cells; and
use the metabolic network and values for the set of selected metabolic parameters or conditions to determine a real-time metabolic flux distribution in the cultured cells.

105. The article of claim 104, wherein the cells are prokaryotic cells, and the instructions are operative to cause the machine to retrieve metabolic network information on the prokaryotic cells from an electronic device and to use the information to process the electronic data.

106. The article of claim 105, wherein the prokaryotic cells are bacterial cells.

107. The article of claim 104, wherein the cells are fungal cells, yeast cells, plant cells or insect cells, and the instructions are operative to cause the machine to retrieve metabolic network information of the cells from an electronic device and to use the information to process the electronic data.

108. The article of claim 104, wherein the cells are eukaryotic cells, and the instructions are operative to cause the machine to retrieve metabolic network information on the eukaryotic cells from an electronic device and to use the information to process the electronic data..

109. The article of claim 108, wherein the cells are mammalian cells.

110. The article of claim 109, wherein the mammalian cells are human cells.

111. The article of claim 104, wherein the data on the metabolic network for the cultured cells comprises a stoichiometry matrix for the cultured cells.

112. The article of claim 111, wherein the stoichiometry matrix comprises a representation of a metabolic network of the cultured cells.

113. The article of claim 111, wherein the stoichiometry matrix defines the presence or absence of metabolic pathway associations.

114. The article of claim 111, wherein the stoichiometry matrix is represented by a stoichiometry coefficient A, wherein A·x=r, and r is a measurement vector representing on-line real-time measurements of the metabolic parameters and x is a flux vector having the units mmol/hour dry cell weight (DCW).

115. The article of claim 1 14, wherein r the measurement vector represents the specific input and output rates of enzymes in a metabolic pathway of the cultured cells.

116. The article of claim 104, wherein the data on the metabolic network for the cultured cells is from at least one of a group consisting of bioinformatics, stoichiometry, genomics, proteomics, metabolomics, microbiology and biochemical pathway and enzyme kinetics knowledge.

117. The article of claim 104, wherein the metabolic network for the selected cell comprise a set of stoichiometric equations for metabolites in the selected cell.

118. The article of claim 104, wherein the instructions are further operative to cause the machine to present the real-time metabolic flux distribution in the selected cell in a display device coupled to the machine.

119. The article of claim 118, wherein the instructions are further operative to cause the machine to present the real-time metabolic flux distribution in a graphical form in the display device.

120. The article of claim 119, wherein the graphical form in the display device shows internal metabolic fluxes over a map of relevant metabolic pathways in the selected cell.

121. The article of claim 104, wherein the instructions are further operative to cause the machine to establish a communication with a local or remote electronic device to retrieve information on metabolic network of cells under culturing stored in said electronic device.

122. The article of claim 118, wherein the instructions are operable in at least one operating system selected from a group consisting of Windows, UNIX, Linux, and MacOS.

123. The article of claim 118, wherein the instructions are further operative to cause the machine to:
obtain at least two different measurements in real time during the culturing;
process the two different measurements to determine a rate of change in a metabolic parameter in real time during the culturing; and
use the rate of change in the metabolic network to determine the real-time metabolic flux distribution in the cultured cells.

124. A system, comprising:
(a) a controllable cell environment for culturing cells, wherein the operating conditions for culturing the cells is controllable in response to a control command;
(b) a sensing subsystem coupled to the controllable cell environment to obtain, in real time during the culturing, measurements associated with culturing of the cells in the controllable cell environment; and
(c) a system controller coupled to the sensing subsystem to receive, in real time during the culturing, the measurements and operable to process the measurements to produce a real-time metabolic flux distribution in the cultured cells.

125. The system of claim 124, wherein the operating conditions for culturing the cells is based on a real-time metabolic flux distribution in the cultured cells.

126. The system of claim 125, further comprising use of the real-time metabolic flux distribution of step (c) to determine the operating conditions for culturing the cells of step (a).

127. The system of claim 124, wherein the controllable cell environment comprises a fermentor or a bioreactor.

128. The system of claim 124, wherein the controllable cell environment comprises a flask, a plate, a capillary tube, a test tube, a biomatrix or an artificial organ.

129. The system of claim 127, wherein the controllable cell environment comprises a plurality of microbioreactors.

130. The system of claim 124, wherein the controllable cell environment comprises a cell growth monitor device.

131. The system of claim 130, wherein the cell growth monitor device measures cell density.

132. The system of claim 131, wherein the cells are cultured in a liquid medium and the cell density is monitored by on-line measurement of optical density of the cell culture.

133. The system of claim 124, wherein the sensing subsystem comprises a device that detects an mRNA transcript.

134. The system of claim 133, wherein the device is configured to operate based on Northern blots.

135. The system of claim 133, wherein the device is configured to operate based on quantitative amplification reactions.

136. The system of claim 133, wherein the device is configured to operate based on hybridization to arrays.

137. The system of claim 124, wherein the sensing subsystem comprises a device that detects and determines the levels of a gas, an organic acid, a polypeptide, a peptide, amino acid, a polysaccharide, a lipid or a combination thereof.

138. The system of claim 137, wherein the device comprises a nuclear magnetic resonance (NMR) device.

139. The system of claim 137, wherein the device comprises a spectrophotometer.

140. The system of claim 137, wherein the device comprises a high performance liquid chromatography (HPLC) device.

141. The system of claim 137, wherein the device comprises a thin layer chromatography device.

142. The system of claim 137, wherein the device comprises a hyperdiffusion chromatography device.

143. The system of claim 137, wherein the device is configured to operate based on an immunological method.

144. The system of claim 137, wherein the organic acid is acetate, butyrate, succinate, oxaloacetate, fumarate, alpha-ketoglutarate, phosphate or a combination thereof.

145. The system of claim 137, wherein the gas is oxygen, methanol, hydrogen, ethanol or a combination thereof.

146. The system of claim 137, wherein the sensing subsystem comprises a device that monitors a primary metabolite, a secondary metabolite or a combination thereof.

147. The system of claim 146, wherein the primary metabolite or secondary metabolite comprises ethanol, methanol, glucose or a combination thereof.

148. The system of claim 137, wherein the sensing subsystem comprises a device that detects an intracellular pH value in the controllable cell environment.

149. The system of claim 137, wherein the sensing subsystem comprises a device that detects and identifies a phenotype.

150. The system of claim 137, wherein the sensing subsystem comprises a capillary array operable to monitor a composition in the selected cell.

151. The system of claim 137, wherein the sensing subsystem comprises a device that retrieves a liquid sample from the controllable cell environment and measures a chemical constituent in the liquid sample.

152. The system of claim 137, wherein the sensing subsystem comprises a device that retrieves a gas sample from the controllable cell environment and measures chemical constituents in the gas sample.

153. The system of claim 124, wherein the system controller comprises:
one or more electronic interfaces coupled to the sensing subsystem to transmit data representing the measurements; and
a computer coupled to the electronic interfaces to receive the data, wherein the computer is programmed to process the data to produce the real-time metabolic flux distribution in the cultured cells.

154. The system of claim 153, wherein the computer is programmed to process the data, in real time, to produce values for a set of selected parameters indicative of real-time metabolic properties of the cultured cells in the controllable cell environment.

155. The system of claim 154, wherein the computer is programmed to retrieve information from at least one database comprising data on a metabolic network for the cultured cells.

156. The system of claim 155, wherein the data on the metabolic network for the cultured cells is from at least one of a group consisting of bioinformatics, stoichiometry, genomics, proteomics, metabolomics, microbiology and biochemical pathway and enzyme kinetics knowledge.

157. The system of claim 155, wherein the computer is programmed to use the metabolic network data and the values for the set of selected parameters indicative of real-time metabolic properties of the cultured cells to determine the real-time metabolic flux distribution in the cultured cells.

158. The system of claim 153, wherein the computer is further programmed to:
obtain at least two different measurements in real time during the cell culturing;
processing the two different measurements to determine a rate of change in a metabolic parameter in real time during the culturing; and
using the rate of change in the metabolic network to determine the real-time metabolic flux distribution in the selected cell during the culturing.

159. The system of claim 153, wherein the computer is configured to operate in at least one operating system selected from a group consisting of Windows, UNIX, Linux and MacOS.

160. The system of claim 153, wherein the system controller further comprises a display device coupled to the computer.

161. The system of claim 153, wherein the computer is further programmed to present the real-time metabolic flux distribution in a graphical form in the display device.

162. The system of claim 161, wherein the computer is further programmed to present the graphical form such that internal metabolic fluxes are shown over a map of relevant metabolic pathways in the selected cell.

163. The system of claim 124, further comprising a cell modification subsystem that operates to modify a genetic composition in a cell in the controllable cell environment in response to the real-time metabolic flux distribution produced by the system controller.

164. The system of claim 155, wherein the data on the metabolic network for the cultured cells comprises a stoichiometry matrix for the cultured cells.

165. The system of claim 164, wherein the stoichiometry matrix comprises a representation of a metabolic network of the cultured cells.

166. The system of claim 164, wherein the stoichiometry matrix defines the presence or absence of metabolic pathway associations.

167. The system of claim 164, wherein the stoichiometry matrix is represented by a stoichiometry coefficient A, wherein A·x=r, and r is a measurement vector representing on-line real-time measurements of the metabolic parameters and x is a flux vector having the units mmol/hour dry cell weight (DCW).

168. The system of claim 167, wherein r the measurement vector represents the specific input and output rates of enzymes in a metabolic pathway of the cultured cells.

169. The system of claim 124, wherein the system controller comprises a computer which is programmed to use a metabolic network model for a selected cell under culturing to generate the metabolic flux distribution.

170. The system of claim 169, wherein the computer is programmed to retrieve information for the metabolic network model from an electronic device.

171. The system of claim 170, wherein the electronic device is a storage device inside the computer.

172. The system of claim 171, wherein the electronic device is a storage device outside the computer and is connected to the computer via a communication link.

173. The system of claim 172, wherein the communication link is established via a computer network.

174. The system of claim 172, wherein the communication link is established via the Internet.

175. The system of claim 170, wherein the electronic device is in another computer linked to the computer.

176. A method for determining the optimal culture conditions for generating a desired product or a desired phenotype in cultured cells comprising:
culturing cells in a controllable cell environment;
measuring at least one metabolic parameter to obtain at least two different measurements in real time during the culturing;
processing the two different measurements to determine a rate of change in the metabolic parameter in real time during the culturing;
applying the rate of change in a set of stoichiometric equations for metabolic characteristics of the cells to determine a real-time metabolic flux distribution in the cells during the culturing; and
adjusting an operating parameter of the controllable cell environment in accordance with the determined real-time metabolic flux distribution to change a culturing condition to modify the metabolic flux distribution during the culturing, thereby optimizing culture conditions for generating a desired product or a desired phenotype.

177. The method as in claim 176, further comprising obtaining information for metabolic flux analysis and using the obtained information in processing the measurements.

178. The method as in claim 177, further comprising obtaining the information for metabolic flux analysis from a database connected via a communication link.

179. The method as in claim 177, wherein the database is an on-line database in a computer server.

180. The method as in claim 179, further comprising accessing the database via the Internet.

181. The method as in claim 177, further comprising accessing a genomic database to obtain the information.

182. The method as in claim 176, further comprising using the real-time metabolic flux distribution to make a modification in a genomic structure of a desired cell.

183. The method as in claim 176, further comprising using the real-time metabolic flux distribution to analyze a property of the cells at physiological level, genomic level, or evolutionary level.

184. The method as in claim 176, further comprising applying selected constraints to the stoichiometric equations to analyze a property of the cells at physiological level, metabolic level, genomic level, or evolutionary level.

185. The method as in claim 176, further comprising applying selected constraints to the stoichiometric equations to select a genomic property of the cells.

186. A method for controlling a computer to perform an on-line metabolic flux analysis for cells under culturing in real time, comprising:
directing the computer to access information on a proper metabolic network model for a selected cell under culturing for determining a metabolic flux distribution of the selected cell;
directing the computer to receive data for determining the metabolic flux distribution;
computing specific rates by using received data;
applying the metabolic network model to the specific rates to determine the metabolic flux distribution;
sending data for the metabolic flux distribution to data files for storage and a computer display device for display;
producing a new metabolic flux distribution when input data is changed; and
when the input data is not changed, directing the computer to wait for a new set of data for determining a new metabolic flux distribution corresponding to the new set of data.

187. The method as in claim 186, wherein the computer is directed to communicate with a linked electronic storage device to access the information on the proper metabolic network model.

188. The method as in claim 187, wherein the computer is linked to the storage device via the Internet.

189. The method as in claim 187, wherein the storage device is another computer.

190. The method as in claim 186, wherein the information includes bioinformatics data on the selected cell.

191. The method as in claim 186, wherein the information includes stoichiometry information on the selected cell.

192. The method as in claim 186, wherein the computer is directed to a data file to receive data obtained in a prior measurement for determining the metabolic flux distribution.

193. The method as in claim 186, wherein the computer is directed to initialize one or more electronic interfaces with sensing devices that are coupled to a cell environment in which cells are cultured to receive real-time data for determining the metabolic flux distribution.

194. A cell made by a method comprising the following steps:
(a) making a modified cell by modifying the genetic composition of a cell;
(b) culturing the modified cell to generate a plurality of modified cells;
(c) measuring at least one metabolic parameter of the cell by monitoring the cell culture of step (b) in real time; and,
(d) analyzing the data of step (c) to determine if the measured parameter differs from a comparable measurement in an unmodified cell under similar conditions, thereby identifying an engineered phenotype in the cell using real-time metabolic flux analysis.

195. The cell of claim 194, wherein the method further comprises the following steps:
providing a template polynucleotide, wherein the template polynucleotide comprises a homologous gene of the cell;
providing a plurality of oligonucleotides, wherein each oligonucleotide comprises a sequence homologous to the template polynucleotide, thereby targeting a specific sequence of the template polynucleotide, and a sequence that is a variant of the homologous gene;
generating progeny polynucleotides comprising non-stochastic sequence variations by replicating the template polynucleotide with the oligonucleotides, thereby generating polynucleotides comprising homologous gene sequence variations.

196. A method for determining a real-time metabolic flux distribution in the cultured cells using an article comprising a machine-readable medium including machine-executable instructions, the instructions being operative to cause a machine to:
electronically interface with a plurality of measuring devices coupled to a controllable cell environment to, in real time, obtain electronic data indicative of a plurality of metabolic parameters or conditions of cell culturing therein;
process the electronic data, in real time, to produce values for a set of selected metabolic parameters or conditions indicative of real-time metabolic properties of the cultured cells in the controllable cell environment;
retrieve information from at least one database comprising data on a metabolic network for the cultured cells; and
use the metabolic network and values for the set of selected metabolic parameters or conditions to determine a real-time metabolic flux distribution in the cultured cells.

197. A cultured cell system having optimal culture conditions for generating a desired product or a desired phenotype made by a method comprising the following steps:
culturing cells in a controllable cell environment;
measuring at least one metabolic parameter to obtain at least two different measurements in real time during the culturing;
processing the two different measurements to determine a rate of change in the metabolic parameter in real time during the culturing;
applying the rate of change in a set of stoichiometric equations for metabolic characteristics of the cells to determine a real-time metabolic flux distribution in the cells during the culturing; and
adjusting an operating parameter of the controllable cell environment in accordance with the determined real-time metabolic flux distribution to change a culturing condition to modify the metabolic flux distribution during the culturing, thereby optimizing culture conditions for generating a desired product or a desired phenotype.

198. A method for identifying proteins by differential labeling of peptides, the method comprising the following steps:
(a) providing a sample comprising a polypeptide;
(b) providing a plurality of labeling reagents which differ in molecular mass but have the same or nearly identical or similar chromatographic retention properties and that have the same or nearly identical or similar ionization and detection properties in mass spectrographic analysis, wherein the differences in molecular mass are distinguishable by mass spectrographic analysis;
(c) fragmenting the polypeptide into peptide fragments by enzymatic digestion or by non-enzymatic fragmentation;
(d) contacting the labeling reagents of step (b) with the peptide fragments of step (c), thereby labeling the peptides with the differential labeling reagents;
(e) separating the peptides by chromatography to generate an eluate;
(f) feeding the eluate of step (e) into a mass spectrometer and quantifying the amount of each peptide and generating the sequence of each peptide by use of the mass spectrometer;
(g) inputting the sequence to a computer program product which compares the inputted sequence to a database of polypeptide sequences to identify the polypeptide from which the sequenced peptide originated.

199. The method of claim 198, wherein the sample of step (a) comprises a cell or a cell extract.

200. The method of claim 198, further comprising providing two or more samples comprising a polypeptide.

201. The method of claim 200, wherein one sample is derived from a wild type cell and one sample is derived from an abnormal or a modified cell.

202. The method of claim 201, wherein the abnormal cell is a cancer cell.

203. The method of claim 198, further comprising purifying or fractionating the polypeptide before the fragmenting of step (c), before the labeling of step (d) or before the chromatography separating of step (e).

204. The method of claim 203, wherein the purifying or fractionating comprises a method selected from the group consisting of size exclusion chromatography, size exclusion chromatography, HPLC, reverse phase HPLC and affinity purification.

205. The method of claim 198, further comprising contacting the polypeptide with a labeling reagent of step (b) before the fragmenting of step (c).

206. The method of claim 198, further comprising contacting the polypeptide with a labeling reagent of step (b) before the fragmenting of step (c).

207. The method of claim 198, wherein the labeling reagent of step (b) comprises the general formulae selected from the group consisting of:
ZAOH and ZBOH, to esterify peptide C-terminals and/or Glu and Asp side chains;
ZANH2 and ZBNH2, to form amide bond with peptide C-terminals and/or Glu and Asp side chains; and
ZACO2H and ZBCO2H. to form amide bond with peptide N-terminals and/or Lys and Arg side chains;
wherein ZA and ZB independently of one another comprise the general formula R-Z1-A1-Z2-A2-Z3-A3-Z4-A4-,
Z1, Z2, Z3, and Z4 independently of one another, are selected from the group consisting of nothing, O, OC(O), OC(S), OC(O)O, OC(O)NR, OC(S)NR, OSiRR1, S, SC(O), SC(S), SS, S(O), S(O2), NR, NRR1+, C(O), C(O)O, C(S), C(S)O, C(O)S, C(O)NR, C(S)NR, SiRR1, (Si(RR1)O)n, SnRR1, Sn(RR1)O, BR(OR1), BRR1, B(OR)(OR1), OBR(OR1), OBRR1, and OB(OR)(OR1), and R and R1 is an alkyl group,
A1, A2, A3, and A4 independently of one another, are selected from the group consisting of nothing or (CRR1)n, wherein R, R1, independently from other R and R1 in Z1 to Z4 and independently from other R and R1 in A1 to A4, are selected from the group consisting of a hydrogen atom, a halogen atom and an alkyl group;
n in Z1 to Z4, independent of n in A1 to A4, is an integer having a value selected from the group consisting of 0 to about 5 1; 0 to about 41; 0 to about 31; 0 to about 21, 0 to about 11 and 0 to about 6.

208. The method of claim 207, wherein the alkyl group is selected from the group consisting of an alkenyl, an alkynyl and an aryl group.

209. The method of claim 207, wherein one or more C—C bonds from (CRR1)n are replaced with a double or a triple bond.

210. The method of claim 207, wherein an R or an R1 group is deleted.

211. The method of claim 207, wherein (CRR1)n is selected from the group consisting of an o-arylene, an m-arylene and a p-arylene, wherein each group has none or up to 6 substituents.

212. The method of claim 207, wherein (CRR1)n is selected from the group consisting of a carbocyclic, a bicyclic and a tricyclic fragment, wherein the fragment has up to 8 atoms in the cycle with or without a heteroatom selected from the group consisting of an O atom, a N atom and an S atom.

213. A method for defining the expressed proteins associated with a given cellular state, the method comprising the following steps:
(a) providing a sample comprising a cell in the desired cellular state;
(b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis;
(c) fragmenting polypeptides derived from the cell into peptide fragments by enzymatic digestion or by non-enzymatic fragmentation;
(d) contacting the labeling reagents of step (b) with the peptide fragments of step (c), thereby labeling the peptides with the differential labeling reagents;
(e) separating the peptides by chromatography to generate an eluate;
(f) feeding the eluate of step (e) into a mass spectrometer and quantifying the amount of each peptide and generating the sequence of each peptide by use of the mass spectrometer;
(g) inputting the sequence to a computer program product which compares the inputted sequence to a database of polypeptide sequences to identify the polypeptide from which the sequenced peptide originated, thereby defining the expressed proteins associated with the cellular state.

214. A method for quantifying changes in protein expression between at least two cellular states, the method comprising the following steps:
(a) providing at least two samples comprising cells in a desired cellular state;
(b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis;
(c) fragmenting polypeptides derived from the cells into peptide fragments by enzymatic digestion or by non-enzymatic fragmentation;
(d) contacting the labeling reagents of step (b) with the peptide fragments of step (c), thereby labeling the peptides with the differential labeling reagents, wherein the labels used in one same are different from the labels used in other samples;
(e) separating the peptides by chromatography to generate an eluate;
(f) feeding the eluate of step (e) into a mass spectrometer and quantifying the amount of each peptide and generating the sequence of each peptide by use of the mass spectrometer;
(g) inputting the sequence to a computer program product which identifies from which sample each peptide was derived, compares the inputted sequence to a database of polypeptide sequences to identify the polypeptide from which the sequenced peptide originated, and compares the amount of each polypeptide in each sample, thereby quantifying changes in protein expression between at least two cellular states.

215. A method for identifying proteins by differential labeling of peptides, the method comprising the following steps:
(a) providing a sample comprising a polypeptide;
(b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis;
(c) fragmenting the polypeptide into peptide fragments by enzymatic digestion or by non-enzymatic fragmentation;
(d) contacting the labeling reagents of step (b) with the peptide fragments of step (c), thereby labeling the peptides with the differential labeling reagents;
(e) separating the peptides by multidimensional liquid chromatography to generate an eluate;
(f) feeding the eluate of step (e) into a tandem mass spectrometer and quantifying the amount of each peptide and generating the sequence of each peptide by use of the mass spectrometer;
(g) inputting the sequence to a computer program product which compares the inputted sequence to a database of polypeptide sequences to identify the polypeptide from which the sequenced peptide originated.

216. A chimeric labeling reagent comprising
(a) a first domain comprising a biotin; and
(b) a second domain comprising a reactive group capable of covalently binding to an amino acid,
wherein the chimeric labeling reagent comprises at least one isotope.

217. A method of comparing relative protein concentrations in a sample comprising
(a) providing a plurality of differential small molecule tags, wherein the small molecule tags are structurally identical but differ in their isotope composition, and the small molecules comprise reactive groups that covalently bind to cysteine or lysine residues or both;
(b) providing at least two samples comprising polypeptides;
(c) attaching covalently the differential small molecule tags to amino acids of the polypeptides;
(d) determining the protein concentrations of each sample in a tandem mass spectrometer; and,
(d) comparing relative protein concentrations of each sample.

218. A method of comparing relative protein concentrations in a sample comprising
(a) providing a plurality of differential small molecule tags, wherein the differential small molecule tags comprise a chimeric labeling reagent comprising (i) a first domain comprising a biotin; and, (ii) a second domain comprising a reactive group capable of covalently binding to an amino acid, wherein the chimeric labeling reagent comprises at least one isotope;
(b) providing at least two samples comprising polypeptides;
(c) attaching covalently the differential small molecule tags to amino acids of the polypeptides;
(d) isolating the tagged polypeptides on a biotin-binding column by binding tagged polypeptides to the column, washing non-bound materials off the column, and eluting tagged polypeptides off the column;
(e) determining the protein concentrations of each sample in a tandem mass spectrometer; and,
(f) comparing relative protein concentrations of each sample.

219. A multidimensional micro liquid chromatography MS/MS (μLC-MS/MS) system comprising three-dimensional (3-D) microcapillary columns for liquid chromatograph (LC) separation of peptides comprising a configuration comprising a reverse phase (RP1) chromatograph, a strong cation exchange (SCX) chromatograph and a reverse phase (RP2) resin chromatograph.

220. The multidimensional micro liquid chromatography MS/MS (μLC-MS/MS) system of claim 119, wherein the system is configured with the components of the system are in the following order: a reverse phase (RP1) chromatograph, followed by a strong cation exchange (SCX) chromatograph, followed by a reverse phase (RP2) resin chromatograph.

221. A method for separating peptides comprising the following steps:
(a) providing a multidimensional micro liquid chromatography MS/MS (μLC-MS/MS) system comprising three-dimensional (3-D) microcapillary columns for liquid chromatograph (LC) separation of peptides comprising a configuration comprising a reverse phase (RP1) chromatograph column, a strong cation exchange (SCX) chromatograph column and a reverse phase (RP2) resin chromatograph column;
(b) providing a mixture of peptides; and
(c) loading onto and running the peptides through the multidimensional micro liquid chromatography MS/MS (μLC-MS/MS) system.

222. The method of claim 221, wherein the system is configured with the components of the system are in the following order: a reverse phase (RP1) chromatograph column, followed by a strong cation exchange (SCX) chromatograph column, followed by a reverse phase (RP2) resin chromatograph column.

223. The method of claim 221, wherein a discrete fraction of the absorbed peptides are displaced from the reverse phase (RP2) resin to the strong cation exchange (SCX) chromatograph column using a reverse phase gradient Xn−Xn+1%.

224. The method of claim 223, wherein the displaced fraction of peptides are retained onto the strong cation exchange (SCX) chromatograph column and then sub-fractionated from the strong cation exchange (SCX) chromatograph column onto the reverse phase (RP2) resin column using a step gradient of salt, wherein part of the peptides are eluted and retained on the reverse phase (RP1) chromatograph column while contaminating salts and buffers are washed through.

225. The method of claim 223, wherein the sub-fractionated peptides are then separated on the RP1 column using the same reverse phase gradient Xn−Xn+1%.

226. The method of claim 225, wherein masses and sequences of separated and eluted peptides are directly detected by a tandem mass spectrometer.

227. The method of claim 225, wherein the process is repeated using increasing salt concentration to displace additional sub-fractions from the SCX column following each step by a reverse phase gradient.

228. The method of claim 225, wherein upon the completion of the whole sequence of salt steps, the process is repeated, employing a higher reverse phase gradient (Xn+1−Xn+2%, Xn+2>Xn+1, n=0, 1, 2, 3 . . . , X1=0).
